(19) United States
(12) Patent Application Publication
(10) Pub. No.: US 2022/0017715 A1
(43) Pub. Date: Jan. 20, 2022

(54) Compositions and Methods for Efficacy Enhancement of T-Cell Based Immunotherapy

(71) Applicants: DOW GLOBAL TECHNOLOGIES LLC, Midland, MI (US); DOW BRASIL SUDESTE INDUSTRIAL LTDA., Sao Paulo/Sp (BR); ROHM AND HAAS COMPANY, Collegeville, PA (US); YALE UNIVERSITY, New Haven, CT (US)

(72) Inventors: Sidi Chen, Milford, CT (US); Matthew Dong, San Francisco, CA (US)

(21) Appl. No.: 16/625,542

(22) PCT Filed: Jun. 22, 2018
(86) PCT No.: PCT/US2018/038966

§ 371 Date: Dec. 20, 2019

Related U.S. Application Data
(60) Provisional application No. 62/524,148, filed on Jun. 23, 2017.

(51) Int. Cl. C08J 7/048; C08J 7/04; C09D 7/62; C09D 175/08

(52) U.S. Cl. CPC C08J 7/048; C08J 7/0427; C09D 7/62; C08J 2475/04; C08J 2323/08; C08J 2323/14; C09D 175/08

(57) ABSTRACT

The present invention includes compositions and methods for enhancing T cell based immunotherapy. In certain aspects, the invention includes modified T cells and inhibitors of Dhx37 for use in enhancing T cell based immunotherapy and treating cancer.

### CROSS-REFERENCE TO RELATED APPLICATION

This application claims priority to U.S. Provisional Application No. 62/524,148, filed Jun. 23, 2017, which is hereby incorporated herein by reference in its entirety.

### STATEMENT REGARDING FEDERALLY SPONSORED RESEARCH OR DEVELOPMENT

This invention was made with government support under CA121974, CA209992, CA196530, and GM007205 awarded by the National Institutes of Health. The government has certain rights in the invention.

### BACKGROUND OF THE INVENTION

CD8+ T cells play a central role in maintaining cellular integrity of the body by mounting cell-mediated adaptive immune responses against intracellular pathogens and tumors. Selective activation of pathogen-specific CD8+ T cells is mediated by T cell receptor (TCR) recognition of cognate antigen on surface major histocompatibility complex (MHC) class I (MHC-I), which results in T cell proliferation, cytokine secretion, and selective killing of target cells. Defects in this cell population can lead to recurrent infections or cancer, while dysregulated activation of CD8+ T cells can result in immunopathology, and even severe autoimmunity.

CD8+ T cells have become the central focus of new cancer therapeutics due to their specificity for intracellular antigens and their role in cell-mediated immune responses. The most potent drugs that have recently been developed are immune checkpoint modulators. This new class of drugs enhances the anti-tumor response of CD8+ T cells by neutralizing the activity of CTLA-4 or PD-1. Blocking the activity of CTLA-4 permits the activation of naive CD8+ T cells in the absence of sufficient antigen. Inhibiting PD-1 activity can reinvigorate exhausted CD8+ T cells to proliferate and kill malignant cells in a subset of cancer patients. These drugs have been shown to be effective in treating multiple cancer types, including melanoma and lung cancer. Ongoing studies are being conducted looking at the efficacy of these drugs used either as monotherapy or in combinations. Further studies have identified 4-1BB, CD27, CD28, ICOS, LAG3, OX-40, TIM3, and VISTA for potential checkpoint modulation. Newer therapeutics have adapted CD8+ T cell machinery to activate under the control of a transgenically expressed chimeric antigen receptor (CAR-T). This method had success in treating hematopoietic malignancies.

Although checkpoint blockade and CAR-T immunotherapies have been shown to be effective when conventional therapies have failed, these modes of therapy still have large potential for improvement, as a large fraction of patients do not respond or have undesired side effects. More systematic approaches will allow for the identification of novel regulators of T cell functions to better enhance the body's anti-tumor response, perhaps in an orthogonal and/or complementary manner to checkpoint inhibitors.

Studies using gene-set specific RNAi/shRNA libraries have been used to identify novel genes that enhance CD8+ T cell function and cytokine production. These molecular tools operate by suppressing the translation of targeted mRNA through complementary binding, but the effects of RNAi are limited by the expression levels of the targeted mRNA, as well as the introduced small interfering RNA.

The development and application of CRISPR technologies have dramatically enhanced the ability to perform genome editing. High-throughput CRISPR screens have been developed and utilized for discovery of novel genes in multiple applications. Application of CRISPR targeting in T cells is the first step towards manipulating the T cell genome, which, together with the screening technology, leads to the hypothesis that high-throughput genetic screening will open the door for unbiased discovery of key factors in T cell biology in a massively parallel manner. However, large-scale CRISPR perturbation of T cells has not been reported, possibly due to multiple technological obstacles, the complexity of lymphocyte repertoires, the tissue architecture of lymphoid or non-lymphoid organs, or the tumor microenvironment.

There is a need in the art for compositions and methods for enhancing T cell based immunotherapies. The present invention satisfies this need.

### SUMMARY OF THE INVENTION

As described herein, the present invention relates to compositions and methods for enhancing T cell based immunotherapy, performing adoptive cell transfer, and treating cancer.

In one aspect, the invention includes a method of enhancing T cell based immunotherapy in a subject. The method comprises administering to the subject in need thereof a genetically modified T cell, wherein a gene selected from the group consisting of Dhx37, Lyn, Slc35c1, Lexm, Fam103a1 and Odc1 has been mutated in the T cell.

In another aspect, the invention includes a method of performing adoptive cell transfer therapy in a subject. The method comprises administering to the subject in need thereof a genetically modified T cell, wherein a gene selected from the group consisting of Dhx37, Lyn, Slc35c1, Lexm, Fam103a1 and Odc1 has been mutated in the T cell.

In yet another aspect, the invention includes a method of treating cancer in a subject in need thereof. The method comprises administering to the subject a genetically modified T cell wherein a gene selected from the group consisting of Dhx37, Lyn, Slc35c1, Lexm, Fam103a1 and Odc1 has been mutated in the T cell.

In still another aspect, the invention includes a method of treating cancer in a subject in need thereof. The method comprises administering to the subject a therapeutically effective amount of an inhibitor of Dhx37.

Another aspect of the invention includes a method of treating cancer in a subject in need thereof comprising administering to the subject a therapeutically effective amount of an inhibitor of a gene or gene product selected from the group consisting of Lyn, Slc35c1, Lexm, Fam103a1 and Odc.

Yet another aspect of the invention includes a method of generating a genetically modified T cell for use in immunotherapy. The method comprises administering to a naïve T cell a vector comprising a first sgRNA complementary to a first nucleotide sequence of a Dhx37 gene and a second sgRNA complementary to a second nucleotide sequence of the Dhx37 gene.

Still another aspect of the invention includes a method of generating a genetically modified T cell for use in immunotherapy. The method comprises administering to a naïve T cell a vector comprising a first sgRNA complementary to a first nucleotide sequence of a gene selected from the group consisting of Lyn, Slc35c1, Lexm, Fam103a1 and Odc and a second sgRNA complementary to a second nucleotide sequence of a gene selected from the group consisting of Lyn, Slc35c1, Lexm, Fam103a1 and Odc.

In another aspect, the invention includes a composition comprising a genetically modified T cell wherein the Dhx37 gene has been mutated. In yet another aspect, the invention includes a composition comprising a genetically modified T cell wherein a gene selected from the group consisting of Lyn, Slc35c1, Lexm, Fam103a1 and Odc has been mutated. In still another aspect, the invention includes a composition comprising an inhibitor of Dhx37, wherein the inhibitor is selected from the group consisting of an antibody, an siRNA, and a CRISPR system.

Another aspect of the invention includes a kit comprising an inhibitor of Dhx37, wherein the inhibitor is selected from the group consisting of an antibody, an siRNA, and a CRISPR system, and instructional material for use thereof. Yet another aspect of the invention includes a kit comprising a plurality of sgRNAs comprising the nucleotide sequences selected from the group consisting of SEQ ID NOs: 11-3020 and instructional material for use thereof.

In various embodiments of the above aspects or any other aspect of the invention delineated herein, the T cell is selected from the group consisting of a CD8+, a CD4+, a T regulatory (Treg) cell and a Chimeric Antigen Receptor (CAR)-T cell.

In one embodiment, at least one additional gene has been mutated in the T cell. In one embodiment, the at least one additional gene is selected from the group consisting of Dhx37, Lyn, Slc35c1, Lexm, Fam103a1 and Odc1.

In one embodiment, the subject is a human. In one embodiment, the method further comprises administering an additional treatment to the subject. In one embodiment, the additional treatment is selected from the group consisting of an immune checkpoint inhibitor, a PD-1 inhibitor, and a CTLA-4 inhibitor.

In one embodiment, the inhibitor is selected from the group consisting of an antibody, an siRNA, and a CRISPR system. In one embodiment, the CRISPR system comprises a Cas9, and at least one sgRNA complementary to Dhx37.

In one embodiment, the sgRNA comprises a nucleotide sequence selected from the group consisting of SEQ ID NOs: 1-10. In one embodiment, the sgRNA comprises a nucleotide sequence selected from the group consisting of SEQ ID NOs: 11-820. In one embodiment, the antibody recognizes and binds to at least one amino acid sequence selected from the group consisting of SEQ ID NOs: 3022-3031.

In one embodiment, the method further comprises administering to the subject an inhibitor of a gene or gene product selected from the group consisting of Lyn, Slc35c1, Lexm, Fam103a1 and Odc. In one embodiment, the CRISPR system comprises a Cas9, and at least one sgRNA complementary to a gene selected from the group consisting of Lyn, Slc35c1, Lexm, Fam103a1 and Odc. In one embodiment, the sgRNA comprises a nucleotide sequence selected from the group consisting of SEQ ID NOs: 821-3020.

In one embodiment, the first sgRNA nucleotide sequence is selected from the group consisting of SEQ ID NOs: 1-10 and the second sgRNA nucleotide sequence is selected from the group consisting of SEQ ID NOs: 1-10. In one embodiment, the first sgRNA nucleotide sequence is selected from the group consisting of SEQ ID NOs: 11-820 and the second sgRNA nucleotide sequence is selected from the group consisting of SEQ ID NOs: 11-820. In one embodiment, the first sgRNA nucleotide sequence is selected from the group consisting of SEQ ID NOs: 821-3020 and the second sgRNA nucleotide sequence is selected from the group consisting of SEQ ID NOs: 821-3020.

### BRIEF DESCRIPTION OF THE DRAWINGS

The following detailed description of specific embodiments of the invention will be better understood when read in conjunction with the appended drawings. For the purpose of illustrating the invention, there are shown in the drawings exemplary embodiments. It should be understood, however, that the invention is not limited to the precise arrangements and instrumentalities of the embodiments shown in the drawings.

FIGS. 1A-1G are a series of plots and images depicting a T cell knockout vector, a genome-scale library and genetic screen for trafficking and survival in CD8+ T cells with diverse TCRs. FIG. 1A shows schematics of the design of a T cell CRISPR knockout vector, which contains an sgRNA expression cassette and a Thy1.1 expression cassette. FIG. 1B shows schematics of an experiment involving library cloning, virus production, naive Cas9 CD8+ T cell isolation and infection, adoptive transfer, and genome-scale CRISPR library (MKO) targeted CD8+T_{eff} cell survival analysis in organs by high-throughput sgRNA sequencing. Organs collected include the liver, pancreas, lung, muscle and brain as representative non-lymphoid organs, and the spleen and several types of lymph nodes (LNs) as lymphoid organs. The LNs collected include three groups: skin draining lymph nodes (sLNs) that were comprised of inguinal, axillary, and brachial lymph nodes; cervical lymph nodes (cLNs) were comprised of the 6 superficial lymph nodes; and abdominal lymph nodes (aLNs) were comprised of the mesenteric and the pancreatic lymph nodes. FIG. 1C is a set of FACS plots of naive Cas9 CD8+ T cell infectivity with MKO lentivirus by Thy1.1 surface staining showing a population of transduced T cells with a significantly elevated Thy1.1 expression compared to untransduced cells. FIG. 1D is a series of pie charts of sgRNA compositions in representative organs. SgRNAs that comprised ≥2% of total reads for each sample are shown, with the remaining reads classified as “Other.” For clarity, only the gene names associated with each sgRNA are shown. Monoclonal (one major clone), oligoclonal (2 to 10 major clones each with ≥2% of total reads) and polyclonal (more than 10 clones with 2% or more reads) compositions of T cell mutants exist in various organs such as LN, spleen, liver, pancreas, lung, brain and muscle. FIG. 1E is a waterfall plot of the top sgRNAs across all organs ranked by number of organs being enriched in (FDR<0.5%). Inset shows all sgRNAs significantly enriched in ≥20% of organ samples. FIG. 1F is a barplot of the number of genes with 0, 1, 2 or 3 independent sgRNAs that were significantly enriched in at least one organ sample (FDR<0.5%). A total of 115 genes were found to have at least 2 independent sgRNAs enriched. Cd247, Bpifb3, and Tsc2 were found to have 3 independent enriched sgRNAs. FIG. 1G is Venn diagram of the three enrichment criteria to identify the top gene hits (≥2% read abundance in one sample (n=227), significant in ≥20% of samples (considering all associated sgRNAs) (n=118), and ≥2 independent enriched sgRNAs (n=115)). A total of 11 genes satisfied all three criteria (Apc, Cd247, Csnk1a1, Fam103a1, Fam134b, Nf1, Pdcd1, Phf21a, Prkar1a, Rab11b, and Tsc2).

FIGS. 2A-2E are a series of plots and images illustrating a genome-scale screen for trafficking and survival with effector CD8+ T cells with transgenic, clonal TCR. FIG. 2A shows the schematics of an experiment involving crossing an OT-I mouse to a Cas9 mouse, naive CD8+ T cell isolation from OT-I; Cas9 mice, CD8+ T cell transduction, adoptive transfer into mice, and MKO-transduced OT-I; Cas9 CD8+T_{eff} cell survival analysis in organs by high-throughput sgRNA sequencing. Organs collected include the liver, pancreas, lung, muscle and brain as representative non-lymphoid organs, and the spleen and several types of lymph nodes (sLNs, cLNs and aLNs). FIG. 2B is a waterfall plot of the top sgRNAs across all organs ranked by number of organs being enriched in (FDR<0.5%). A total of 27 sgRNAs were found to be significant in ≥20% of samples. FIG. 2C is a barplot of the number of genes with 0, 1, or 2 independent sgRNAs that were significantly enriched in at least one organ sample (FDR<0.5%). A total of 4 genes were found to have 2 independent sgRNAs enriched. Cd247, Bpifb3, and Tsc2 were found to have 3 independent enriched sgRNAs. FIG. 2D is a Venn diagram of the three enrichment criteria to identify the top gene hits (≥2% read abundance in one sample (n=99), significant in ≥20% of samples (considering all associated sgRNAs) (n=27), and ≥2 independent enriched sgRNAs (n=4)). The sets of ≥20% of samples and ≥2 independent enriched sgRNAs were contained in the set of ≥2% read abundance in one sample. A total of 3 genes satisfied all three criteria. These genes were Pdcd1, Slc35c1, and Stradb. FIG. 2E is a Venn diagram comparing the hits from the diverse TCR screen and from the clonal TCR screen. 17 genes were found to be significant in ≥2 samples from both datasets. These included 3830406C13Rik, BC055111, Cd247, Gm6927, Hacvr2, Lrp6, Nf1, Olfr1158, Opn3, Pdcd1, Serping1, Slc2a7, Slc35c1, Son, Tsc2, Tspan4, and Zfp82.

FIGS. 3A-3G are a series of plots and images illustrating a genome-scale screen for tumor infiltration with TCR-engineered T_{eff} cells into tumors expressing a cognate model antigen. FIG. 3A is a schematic of an experiment involving naive CD8+ T cell isolation from OT-I; Cas9 mice, CD8+ T cell transduction, adoptive transfer into E0771-mCh-cOVA tumor-bearing Rag1−/− mice, CD8+T_{eff} cell survival and infiltration analysis in tumors of E0771-mCh-cOVA tumor-bearing Rag1−/− mice by FACS and sgRNA sequencing. FIG. 3B shows measurement of antigen presentation in E0771-mCh-cOVA cell lines. E0771 cells were transduced with a lentiviral vector encoding mCherry-2A-cOVA transgene, and multiple clonal lines were generated by single cell cloning. MHC-I-peptide complex (SIINFEKL:H-K2b) was measured by mean fluorescent intensity (MFI) of surface staining using FACS.

FIG. 3C is a growth curve of mammary fat pad tumors from transplanted E0771-mCh-cOVA cells in Rag1−/− mice following different treatments. PBS control (n=3), adoptive transfer of OT-I; Cas9 CD8+T_{eff} cells infected with vector (n=3), and adoptive transfer of OT-I; Cas9 CD8+T_{eff} cells infected with MKO (n=8). Arrow indicates the time of adoptive transfer of MKO or vector transduced OT-I; Cas9 CD8+T_{eff} cells. Endpoint tumor size vector vs PBS, unpaired two-sided t-test, p=0.02; MKO vs PBS, p<0.0001, MKO vs vector, p=0.03. Data are shown as mean±s.e.m. Noted that some error bars were not visible because the absolute value of errors were small. FIG. 3D is a box-dot plot of overall sgRNA library representation in all samples, including cellular libraries of infected OT-I; Cas9 CD8+T_{eff} cells before injection (n=3), and tumors from multiple mice (n=10 mice, 10 total tumors). sgRNA representation is depicted in terms of log 2 rpm. FIG. 3E is a waterfall plot of the top-ranked sgRNAs across all tumors (21 sgRNAs significantly enriched in ≥50% of tumors, FDR<0.5%). Inset, waterfall plot of all sgRNAs that were significantly enriched in ≥20% of tumors. FIG. 3F is a barplot of the number of genes with 0-4 independent sgRNAs that were significantly enriched in at least one organ sample (FDR<0.5%). A total of 26 genes were found to have at least 2 independent sgRNAs enriched. Pdcd1 and Stradb were each found to have 4 independent enriched sgRNAs. FIG. 3G is a Venn diagram of the three enrichment criteria to identify the top gene hits (≥2% read abundance in one sample (n=36), significant in ≥20% of samples (n=220), and ≥2 independent enriched sgRNAs (n=26)). A total of 6 genes satisfied all three criteria (Cd247, Fam103a1, Hacvr2, Pdcd1, Prkar1a, and Stradb).

FIGS. 4A-4F are a series of plots and images illustrating high-throughput identification of genes modulating effector CD8+ T cell degranulation upon encountering tumor antigen. FIG. 4A shows schematics of an experiment involving naive OT-I; Cas9 CD8+ T cells isolated and transduced with MKO lentiviral library, co-cultured with SIINFEKL peptide pulsed E0771 cells (0 or 1 ng/ml), and stained for CD8 and CD107a for CD8+T_{eff} undergoing active degranulation. Stained cells were analyzed, and the top 5% CD107a+ cells were sorted, and subjected to genomic DNA extraction, CRISPR library readout, and screen data analysis. FIG. 4B shows titration of SIINFEKL peptide for MHC-I presentation in E0771 cells. E0771 cells were pulsed with different concentrations of SIINFEKL peptide, and the MHC-I-peptide complex (SIINFEKL: H-K2b) was measured by mean fluorescent intensity (MFI) of surface staining using FACS. FIG. 4C is a histogram showing CD107a+ T cells analyzed from the co-culture of OT-I; Cas9 CD8+ T cells and E0771 cancer cells. The top 5% CD107a+ cells were sorted. A total of three biological replicates were performed.

FIG. 4D is a waterfall plot of the top-ranked sgRNAs across all sorted cell samples (17 sgRNAs significantly enriched in ≥66% of samples, FDR<0.5%). FIG. 4E is a Venn diagram comparing the hits from the in vitro kill assay screen and from the in vivo tumor infiltration study. 3 genes were found to be significant in ≥2 samples from both datasets. These included Dhx37, Lyn, and Odc1. FIG. 4F shows growth curves of mammary fatpad E0771-mCh-cOVA tumors in Rag1−/− mice following different treatments. PBS control (black, n=4), adoptive transfer of OT-I; Cas9 CD8+T_{eff} cells infected with vector (n=4), and adoptive transfer of OT-I; Cas9 CD8+T_{eff} cells infected with sgDhx37 (n=5). Arrow indicates the time of adoptive transfer of MKO or vector transduced OT-I; Cas9 CD8+T_{eff} cells. Data are shown as mean±s.e.m. Right panel: zoomed in view of tumor growth curves from adoptive transfer of sgDhx37 or vector treated OT-I; Cas9 CD8+T_{eff} cells. Adoptive transfer of sgDhx37 OT-I; Cas9 CD8+T_{eff} cells led to significantly reduced tumor burden compared to vector controls. **=adjusted p<0.01, ***=adjusted p<0.001, by two-sided t-test (Benjamini, Krieger and Yekutieli method).

FIGS. 5A-5E are a series of plots and images illustrating single-cell transcriptomics of sgDhx37 OT-I; Cas9 CD8⁺ TILs in E0771-mCh-cOVA tumors. FIG. 5A shows schematics of an experiment involving adoptive transfer of vector or sgDhx37-infected OT-I; Cas9 CD8+ T_{eff} cells into Rag1−/− mice bearing E0771-mCh-cOVA tumors, tumor harvesting after 50 days of growth, FACS for CD3+CD8+ T cells, microfluidic-based approach of reverse-transcription and multistep barcoding library preparation to produce single-cell barcoded DNA droplets, followed by high-throughput sequencing and computational analysis. FIG. 5B shows t-SNE dimensional reduction and visualization of individual tumor-infiltrating CD8+ cells treated with either sgDhx37 (n=191 cells) or vector (n=361). FIG. 5C is a Volcano plot of differentially expressed genes in tumor-infiltrating CD8+ cells treated with sgDhx37 compared to vector control. A total of 137 genes were significantly upregulated in sgDhx37 treated cells (Benjamini-Hochberg adjusted p<0.05), while 215 genes were significantly downregulated in sgDhx37 treated cells (adjusted p<0.05). Top upregulated genes included Rgs16, Nr4a2, and Tox. FIG. 5D shows gene ontology analysis of significantly upregulated genes in sgDhx37-treated tumor-infiltrating CD8+ cells. Several gene ontology categories were significantly enriched (Bonferroni adjusted p<0.05). These included lymphocyte activation, positive regulation of cytokine production, regulation of cell-cell adhesion, regulation of immune effector process, and positive regulation of interferon-gamma production. FIG. 5E shows gene ontology analysis of significantly downregulated genes in sgDhx37-treated tumor-infiltrating CD8+ cells. Several gene ontology categories were significantly enriched (Bonferroni adjusted p<0.05). These included ribosomal small subunit assembly, ribosomal large subunit biogenesis, regulation of reactive oxygen species metabolic process, regulation of cell migration, positive regulation of leukocyte migration, and apoptotic signaling pathway.

FIGS. 6A-6E are a series of plots and images illustrating FACS data for MKO virus titration for screening. FIG. 6A shows schematics of an experiment involving virus production, CD8+ T cell isolation and infection with a genome-scale CRISPR library (MKO), Thy1.1 surface staining, and FACS analysis. FIG. 6B is a series of FACS plots of naive OT-I; Cas9 CD8+ T cells infection with multiple dilution of MKO lentivirus (Thy1.1 gating) using two batches of viruses collected at different time points. FIG. 6C shows overlaid histograms of Thy1.1 expression of Cas9 CD8+ T cells infected T cells with comparable viral titers from two batches of viruses. Shaded histogram represents uninfected control. Histograms depict MKO library virus isolated 48 hours and 72 hours post-transfection. FIG. 6D shows quantification of MKO lentivirus from two batches of virus by surface staining of Thy1.1-infected CD8+ T cells. Data were shown as geometric mean of MFI. FIG. 6E shows quantification of MKO lentivirus from two batches of virus by surface staining of Thy1.1-infected CD8+ T cells. Data were shown as % Thy1.1+ CD8+ T cells.

FIG. 7 is a plot illustrating correlation analysis of sgRNA library representation in all samples from the genome-scale screen for trafficking and survival in CD8+ T cells with diverse TCR. Heatmap of pairwise Pearson correlations of sgRNA library representation across all samples in the first WT screen using Cas9 CD8+ T cells that have a diverse TCR repertoire. Samples included plasmid library (n=1), cellular libraries of pre-injection library-infected naive CD8+ T cells (n=3), and various organs containing CD8+T_{eff} cells from multiple mice 7 days post-injection (n=7 mice, 62 total samples). Correlations were calculated based on log₂ rpm values. Cell and plasmid samples were highly correlated with each other, while organ samples were most correlated with other organ samples.

FIG. 8 is a box-dot plot illustrating overall library sgRNA representation in all samples from the genome-scale screen for trafficking and survival in CD8+ T cells with diverse TCR. Shown is overall sgRNA library representation in all samples, including plasmid library (n=1), cellular libraries of pre-injection library-infected naive CD8+ T cells (n=3), and various organs containing CD8+T_{eff} cells from multiple mice 7 days post-injection (n=7 mice, 62 total samples). SgRNA representation is depicted in terms of log₂ reads per million (rpm). Analyzed tissues include the lymph node (LN), spleen, brain, liver, lung, muscle, and pancreas.

FIG. 9 is a heatmap illustrating correlation analysis of genome-scale CRISPR perturbation of OT-I; Cas9 CD8+ T cell survival in WT mice. Heatmap of pairwise Pearson correlations of sgRNA library representation across all samples in the second WT screen using OT-I; Cas9 CD8+ T cells. Samples were from various organs containing CD8+T_{eff} cells from multiple mice 7 days post-injection (n=10 mice, 70 total samples). Correlations were calculated based on log₂ rpm values.

FIG. 10 is a plot illustrating overall library sgRNA abundance of diverse OT-I; Cas9 CD8+ T cell survival in WT mice. Box-dot plot of overall sgRNA library representation in all samples from various organs containing CD8+T_{eff} cells from multiple mice 7 days post-injection (n=10 mice, 70 total samples). sgRNA representation is depicted in terms of log₂ reads per million (rpm). Analyzed tissues include various lymph nodes (LN), spleen, liver, pancreas, and lung.

FIGS. 11A-11B are a plot and a series of images illustrating representative histology of tumors derived from E0771 cells expressing cOVA antigen in Rag1^{-/-} mice after adoptive transfer. FIG. 11A is a growth curve of subcutaneous tumors from transplanted E0771-mCh-cOVA cells in Rag1^{-/-} mice following different treatments. PBS control (n=1), adoptive transfer of OT-I; Cas9 CD8⁺ T_{eff} cells infected with vector (n=3), and adoptive transfer of OT-I; Cas9 CD8⁺ T_{eff} cells infected with MKO (n=5). Arrow indicates the time of adoptive transfer of MKO or vector transduced OT-I; Cas9 CD8⁺ T_{eff} cells. Error bars for certain data points were invisible because the errors were small. Data are shown as mean±s.e.m. FIG. 11B shows full-slide and high-power histology sections stained by hematoxylin and eosin of tumors derived from E0771 cells expressing cOVA antigen in Rag1^{-/-} mice after different treatment conditions. Top group: tumors in mice that were injected with PBS. Middle group: tumors in mice after adoptive transfer of vector-treated activated OT-I; Cas9 CD8⁺ T_{eff} cells.

Bottom group: tumors in mice after adoptive transfer of MKO mutagenized activated OT-I; Cas9 CD8⁺ T_{eff} cells. In PBS group, tumors were devoid of lymphocytes and showed signatures of rapid proliferation and little cell death. In adoptive transfer groups, tumors were infiltrated by lymphocytes and showed signatures of cell death in large areas. Low magnification image scale bar: 1 mm; high magnification image scale bar: 200 μm.

FIG. 12 is a series of plots illustrating FACS data for setup experiments of MKO mutagenized activated OT-I; Cas9 CD8⁺ T_{eff} cells in Rag1^{-/-} mice with transplanted tumors expressing cOVA antigen. Representative FACS plots of adoptively transferred T_{eff} cells in draining and non-draining LNs (dLN and ndLN, respectively), spleen, lung, and tumor (TILs) from E0771-mCh-cOVA tumor-bearing Rag1^{-/-} mice. MKO is the genome-scale T cell knockout CRISPR library. Numbers indicate percentage of total cells. Top row: FACS plots from PBS-treated mice. Middle row: FACS plots from mice treated with vector-infected OT-I; Cas9 CD8⁺ T cells. Bottom row: FACS plots from mice treated with MKO-infected OT-I; Cas9 CD8⁺ T cells.

FIG. 13 is a heatmap illustrating correlation analysis of genome-scale CRISPR perturbation of OT-I; Cas9 CD8⁺ tumor-infiltrating lymphocytes into Rag1^{-/-} mice with E0771-cOVA tumors. Heatmap of pairwise Pearson correlations of sgRNA library representation across 3 cell libraries prior to injection, and all samples in the tumor infiltration screen (n=10 mice, 10 tumors). Correlations were calculated based on log₂ rpm values. E0771-cOVA cells were transplanted subcutaneously for mice 1-5, and into the mammary fat pad for mice 6-10.

FIG. 14 is a heatmap illustrating differentially expressed genes in sgDhx37-treated CD8⁺ tumor infiltrating lymphocytes compared to vector-treated. Heatmap of top differentially expressed genes (absolute log₂ fold change≥1) in single CD8⁺ tumor infiltrating lymphocytes treated with sgDhx37 or vector control. Values shown are in terms of z-scores (scaled by row/gene).

FIGS. 15A-15DD are a series of tables illustrating the sgRNA sequences targeting human genes of top hits identified in the T cell screens herein, such as sg-DHX37, sg-LEXM, sg-FAM103A1, sg-ODC1, and sg-SLC35C1.

### DETAILED DESCRIPTION

### Definitions

Unless defined otherwise, all technical and scientific terms used herein have the same meaning as commonly understood by one of ordinary skill in the art to which the invention pertains. Although any methods and materials similar or equivalent to those described herein can be used in the practice for testing of the present invention, the preferred materials and methods are described herein. In describing and claiming the present invention, the following terminology will be used.

It is also to be understood that the terminology used herein is for the purpose of describing particular embodiments only, and is not intended to be limiting.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20% or ±10%, more preferably ±5%, even more preferably ±1%, and still more preferably ±0.1% from the specified value, as such variations are appropriate to perform the disclosed methods.

As used herein the term “amount” refers to the abundance or quantity of a constituent in a mixture.

As used herein, the term “bp” refers to base pair.

The term “complementary” refers to the degree of anti-parallel alignment between two nucleic acid strands. Complete complementarity requires that each nucleotide be across from its opposite. No complementarity requires that each nucleotide is not across from its opposite. The degree of complementarity determines the stability of the sequences to be together or anneal/hybridize. Furthermore various DNA repair functions as well as regulatory functions are based on base pair complementarity.

The term “CRISPR/Cas” or “clustered regularly interspaced short palindromic repeats” or “CRISPR” refers to DNA loci containing short repetitions of base sequences followed by short segments of spacer DNA from previous exposures to a virus or plasmid.

Bacteria and archaea have evolved adaptive immune defenses termed CRISPR/CRISPR-associated (Cas) systems that use short RNA to direct degradation of foreign nucleic acids. In bacteria, the CRISPR system provides acquired immunity against invading foreign DNA via RNA-guided DNA cleavage.

The “CRISPR/Cas9” system or “CRISPR/Cas9-mediated gene editing” refers to a type II CRISPR/Cas system that has been modified for genome editing/engineering. It is typically comprised of a “guide” RNA (gRNA) and a non-specific CRISPR-associated endonuclease (Cas9). “Guide RNA (gRNA)” is used interchangeably herein with “short guide RNA (sgRNA)” or “single guide RNA” (sgRNA). The sgRNA is a short synthetic RNA composed of a “scaffold” sequence necessary for Cas9-binding and a user-defined ˜20 nucleotide “spacer” or “targeting” sequence which defines the genomic target to be modified. The genomic target of Cas9 can be modified by changing the targeting sequence present in the sgRNA.

The term “cleavage” refers to the breakage of covalent bonds, such as in the backbone of a nucleic acid molecule or the hydrolysis of peptide bonds. Cleavage can be initiated by a variety of methods, including, but not limited to, enzymatic or chemical hydrolysis of a phosphodiester bond. Both single-stranded cleavage and double-stranded cleavage are possible. Double-stranded cleavage can occur as a result of two distinct single-stranded cleavage events. DNA cleavage can result in the production of either blunt ends or staggered ends. In certain embodiments, fusion polypeptides can be used for targeting cleaved double-stranded DNA.

A “disease” is a state of health of an animal wherein the animal cannot maintain homeostasis, and wherein if the disease is not ameliorated then the animal's health continues to deteriorate. In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

“Effective amount” or “therapeutically effective amount” are used interchangeably herein, and refer to an amount of a compound, formulation, material, or composition, as described herein effective to achieve a particular biological result or provides a therapeutic or prophylactic benefit. Such results may include, but are not limited to, anti-tumor activity as determined by any means suitable in the art.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

As used herein “endogenous” refers to any material from or produced inside an organism, cell, tissue or system.

The term “expression” as used herein is defined as the transcription and/or translation of a particular nucleotide sequence driven by its promoter.

“Expression vector” refers to a vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, such as cosmids, plasmids (e.g., naked or contained in liposomes) and viruses (e.g., Sendai viruses, lentiviruses, retroviruses, adenoviruses, and adeno-associated viruses) that incorporate the recombinant polynucleotide.

“Homologous” as used herein, refers to the subunit sequence identity between two polymeric molecules, e.g., between two nucleic acid molecules, such as, two DNA molecules or two RNA molecules, or between two polypeptide molecules. When a subunit position in both of the two molecules is occupied by the same monomeric subunit; e.g., if a position in each of two DNA molecules is occupied by adenine, then they are homologous at that position. The homology between two sequences is a direct function of the number of matching or homologous positions; e.g., if half (e.g., five positions in a polymer ten subunits in length) of the positions in two sequences are homologous, the two sequences are 50% homologous; if 90% of the positions (e.g., 9 of 10), are matched or homologous, the two sequences are 90% homologous.

“Identity” as used herein refers to the subunit sequence identity between two polymeric molecules particularly between two amino acid molecules, such as, between two polypeptide molecules. When two amino acid sequences have the same residues at the same positions; e.g., if a position in each of two polypeptide molecules is occupied by an Arginine, then they are identical at that position. The identity or extent to which two amino acid sequences have the same residues at the same positions in an alignment is often expressed as a percentage. The identity between two amino acid sequences is a direct function of the number of matching or identical positions; e.g., if half (e.g., five positions in a polymer ten amino acids in length) of the positions in two sequences are identical, the two sequences are 50% identical; if 90% of the positions (e.g., 9 of 10), are matched or identical, the two amino acids sequences are 90% identical.

As used herein, an “instructional material” includes a publication, a recording, a diagram, or any other medium of expression that can be used to communicate the usefulness of the compositions and methods of the invention. The instructional material of the kit of the invention may, for example, be affixed to a container that contains the nucleic acid, peptide, and/or composition of the invention or be shipped together with a container which contains the nucleic acid, peptide, and/or composition. Alternatively, the instructional material may be shipped separately from the container with the intention that the instructional material and compound be used cooperatively by the recipient.

“Isolated” means altered or removed from the natural state. For example, a nucleic acid or a peptide naturally present in a living animal is not “isolated,” but the same nucleic acid or peptide partially or completely separated from the coexisting materials of its natural state is “isolated.” An isolated nucleic acid or protein can exist in substantially purified form, or can exist in a non-native environment such as, for example, a host cell.

The term “knockdown” as used herein refers to a decrease in gene expression of one or more genes.

The term “knockout” as used herein refers to the ablation of gene expression of one or more genes.

A “lentivirus” as used herein refers to a genus of the Retroviridae family. Lentiviruses are unique among the retroviruses in being able to infect non-dividing cells; they can deliver a significant amount of genetic information into the DNA of the host cell, so they are one of the most efficient methods of a gene delivery vector. HIV, SIV, and FIV are all examples of lentiviruses. Vectors derived from lentiviruses offer the means to achieve significant levels of gene transfer in vivo.

By the term “modified” as used herein, is meant a changed state or structure of a molecule or cell of the invention. Molecules may be modified in many ways, including chemically, structurally, and functionally. Cells may be modified through the introduction of nucleic acids.

By the term “modulating,” as used herein, is meant mediating a detectable increase or decrease in the level of a response in a subject compared with the level of a response in the subject in the absence of a treatment or compound, and/or compared with the level of a response in an otherwise identical but untreated subject. The term encompasses perturbing and/or affecting a native signal or response thereby mediating a beneficial therapeutic response in a subject, preferably, a human.

A “mutation” as used herein is a change in a DNA sequence resulting in an alteration from a given reference sequence (which may be, for example, an earlier collected DNA sample from the same subject). The mutation can comprise deletion and/or insertion and/or duplication and/or substitution of at least one deoxyribonucleic acid base such as a purine (adenine and/or thymine) and/or a pyrimidine (guanine and/or cytosine). Mutations may or may not produce discernible changes in the observable characteristics (phenotype) of an organism (subject).

By “nucleic acid” is meant any nucleic acid, whether composed of deoxyribonucleosides or ribonucleosides, and whether composed of phosphodiester linkages or modified linkages such as phosphotriester, phosphoramidate, siloxane, carbonate, carboxymethylester, acetamidate, carbamate, thioether, bridged phosphoramidate, bridged methylene phosphonate, phosphorothioate, methylphosphonate, phosphorodithioate, bridged phosphorothioate or sulfone linkages, and combinations of such linkages. The term nucleic acid also specifically includes nucleic acids composed of bases other than the five biologically occurring bases (adenine, guanine, thymine, cytosine and uracil).

In the context of the present invention, the following abbreviations for the commonly occurring nucleic acid bases are used. “A” refers to adenosine, “C” refers to cytosine, “G” refers to guanosine, “T” refers to thymidine, and “U” refers to uridine.

Unless otherwise specified, a “nucleotide sequence encoding an amino acid sequence” includes all nucleotide sequences that are degenerate versions of each other and that encode the same amino acid sequence. The phrase nucleotide sequence that encodes a protein or an RNA may also include introns to the extent that the nucleotide sequence encoding the protein may in some version contain an intron(s).

The term “oligonucleotide” typically refers to short polynucleotides, generally no greater than about 60 nucleotides. It will be understood that when a nucleotide sequence is represented by a DNA sequence (i.e., A, T, G, C), this also includes an RNA sequence (i.e., A, U, G, C) in which “U” replaces “T”.

“Parenteral” administration of an immunogenic composition includes, e.g., subcutaneous (s.c.), intravenous (i.v.), intramuscular (i.m.), or intrasternal injection, or infusion techniques.

The term “polynucleotide” as used herein is defined as a chain of nucleotides. Furthermore, nucleic acids are polymers of nucleotides. Thus, nucleic acids and polynucleotides as used herein are interchangeable. One skilled in the art has the general knowledge that nucleic acids are polynucleotides, which can be hydrolyzed into the monomeric “nucleotides.” The monomeric nucleotides can be hydrolyzed into nucleosides. As used herein polynucleotides include, but are not limited to, all nucleic acid sequences which are obtained by any means available in the art, including, without limitation, recombinant means, i.e., the cloning of nucleic acid sequences from a recombinant library or a cell genome, using ordinary cloning technology and PCR™, and the like, and by synthetic means. Conventional notation is used herein to describe polynucleotide sequences: the left-hand end of a single-stranded polynucleotide sequence is the 5′-end; the left-hand direction of a double-stranded polynucleotide sequence is referred to as the 5′-direction.

As used herein, the terms “polypeptide,” “peptide,” and “protein” are used interchangeably, and refer to a compound comprised of amino acid residues covalently linked by peptide bonds. A protein or peptide must contain at least two amino acids, and no limitation is placed on the maximum number of amino acids that can comprise a protein's or peptide's sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. The polypeptides include natural peptides, recombinant peptides, synthetic peptides, or a combination thereof.

The term “promoter” as used herein is defined as a DNA sequence recognized by the synthetic machinery of the cell, or introduced synthetic machinery, required to initiate the specific transcription of a polynucleotide sequence.

A “sample” or “biological sample” as used herein means a biological material from a subject, including but is not limited to organ, tissue, exosome, blood, plasma, saliva, urine and other body fluid. A sample can be any source of material obtained from a subject.

As used herein, the terms “sequencing” or “nucleotide sequencing” refer to determining the order of nucleotides (base sequences) in a nucleic acid sample, e.g. DNA or RNA. Many techniques are available such as Sanger sequencing and high-throughput sequencing technologies (also known as next-generation sequencing technologies) such as Illumina's HiSeq and MiSeq platforms or the GS FLX platform offered by Roche Applied Science.

The term “subject” is intended to include living organisms in which an immune response can be elicited (e.g., mammals). A “subject” or “patient,” as used therein, may be a human or non-human mammal. Non-human mammals include, for example, livestock and pets, such as ovine, bovine, porcine, canine, feline and murine mammals. Preferably, the subject is human.

A “target site” or “target sequence” refers to a genomic nucleic acid sequence that defines a portion of a nucleic acid to which a binding molecule may specifically bind under conditions sufficient for binding to occur.

As used herein, the term “T cell receptor” or “TCR” refers to a complex of membrane proteins that participate in the activation of T cells in response to the presentation of antigen. The TCR is responsible for recognizing antigens bound to major histocompatibility complex molecules. TCR is composed of a heterodimer of an alpha (α) and beta (β) chain, although in some cells the TCR consists of gamma and delta (γ/δ) chains. TCRs may exist in α/β and γ/δ forms, which are structurally similar but have distinct anatomical locations and functions. Each chain is composed of two extracellular domains, a variable and constant domain. In some embodiments, the TCR can be modified on any cell comprising a TCR, including, for example, a helper T cell, a cytotoxic T cell, a memory T cell, regulatory T cell, natural killer T cell, and/or gamma delta T cell.

The term “therapeutic” as used herein means a treatment and/or prophylaxis. A therapeutic effect is obtained by suppression, remission, or eradication of a disease state.

The term “transfected” or “transformed” or “transduced” as used herein refers to a process by which exogenous nucleic acid is transferred or introduced into the host cell. A “transfected” or “transformed” or “transduced” cell is one that has been transfected, transformed or transduced with exogenous nucleic acid. The cell includes the primary subject cell and its progeny.

To “treat” a disease as the term is used herein, means to reduce the frequency or severity of at least one sign or symptom of a disease or disorder experienced by a subject.

A “vector” is a composition of matter which comprises an isolated nucleic acid and which can be used to deliver the isolated nucleic acid to the interior of a cell. Numerous vectors are known in the art including, but not limited to, linear polynucleotides, polynucleotides associated with ionic or amphiphilic compounds, plasmids, and viruses. Thus, the term “vector” includes an autonomously replicating plasmid or a virus. The term should also be construed to include non-plasmid and non-viral compounds which facilitate transfer of nucleic acid into cells, such as, for example, polylysine compounds, liposomes, and the like. Examples of viral vectors include, but are not limited to, Sendai viral vectors, adenoviral vectors, adeno-associated virus vectors, retroviral vectors, lentiviral vectors, and the like.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### DESCRIPTION

In the present study, multiple genome-scale in vivo and in vitro CRISPR screens of CD8⁺ cytotoxic T cells were performed to dissect their phenotypes, and quantitative maps of genetic factors modulating important immunological processes such as trafficking, survival, degranulation and tumor infiltration of CD8+ T cells were generated. Dhx37 was one of the top candidates that emerged from multiple screens. Herein, it was demonstrated that targeting this gene with CRISPR leads to significantly enhanced anti-tumor activity. Dhx37 was also mechanistically linked to altered transcriptomes of immunomodulatory and effector genes in tumor-infiltrating lymphocytes (TILs) using single-cell RNAseq.

The screen was performed in two settings of immunotherapy to assay the abilities of OT-I; Cas9 CD8⁺ effector T cells to infiltrate the tumors and to kill cancer cells upon TCR-antigen encounter. These screens converged on the RNA helicase, Dhx37, which has not been associated with T cell function previously. Engineered OT-I; Cas9 CD8⁺ effector T cells with sgRNAs targeting Dhx37 (sgDhx37) had significantly enhanced anti-tumor activity, resulted in reduced tumor burden, and suppressed relapse in a breast cancer model in mice. Single-cell RNA-sequencing profiled the heterogeneous transcriptomes of sgDhx37 TILs, revealing strong signatures of alterations in immune modulating and effector transcripts, including lymphocyte cell adhesion, interferon-gamma pathway, cytokine production and immune effector genes. These data collectively indicate that Dhx37 inhibition is a novel avenue for immunotherapy, potentially alone or in combination with existing checkpoint blockade agents, and could be rationalized to enhance chimeric antigen receptor (CAR) T cell efficacy.

The present invention provides, in one aspect, compositions and methods for enhancing T cell based immunotherapy. In certain embodiments, the invention provides modified T cells and inhibitors of Dhx37 for use in enhancing T cell based immunotherapy and/or treating cancer.

### Compositions

In one aspect, the invention includes a genetically modified T cell wherein a gene selected from the group consisting of Dhx37, Lyn, Slc35c1, Lexm, Fam103a1 and Odc1 has been mutated. In one embodiment, the invention includes a genetically modified T cell wherein the Dhx37 gene has been mutated. The genetically modified T cell can be for use in enhancing T cell based immunotherapy and treating cancer, and can be generated by the methods described herein. The T cell can be of any subtype, including but not limited to CD8+, CD4+, T regulatory (Treg) cells, and CAR-T cells. Additional genes can be mutated in the T cell. In other words, the invention includes a T cell wherein a single gene or multiple genes are mutated. Combinations of genes that can be mutated, include but are not limited to, Dhx37, Lyn, Slc35c1, Lexm, Fam103a1 and Odc1.

In another aspect, the invention includes an inhibitor of Dhx37. By ‘inhibitor of Dhx37” is meant any compound, construct or other that blocks function or production of Dhx37 at the DNA, RNA, or protein level. This can include but is not limited to any drug, small molecule, antibody, siRNA, or CRISPR system. In one aspect, a CRISPR system comprising a Cas9, and at least one sgRNA complementary to Dhx37, can be used to inhibit Dhx37. In certain embodiments, the sgRNAs are complementary to Dhx37. In certain embodiments, the sgRNA comprises a nucleotide sequence selected from the group consisting of SEQ ID NOs: 1-10. In certain embodiments, the sgRNA comprises a nucleotide sequence selected from the group consisting of SEQ ID NOs: 11-820.

TABLE 1
Mouse sgRNAs

| Mouse sgRNA Name | Sequence | SEQ ID NO: |
|---|---|---|
| mm52368_Dhx37 | AAGTTGCCTACCTATAGCAG | SEQ ID NO: 1 |
| mm52369_Dhx37 | CCTGCTTCGTAGAGAAACTG | SEQ ID NO: 2 |
| mm52370_Dhx37 | ACCAACCTAGGACCAGCACA | SEQ ID NO: 3 |
| mm52371_Dhx37 | ACCTGTTACAGGTTGAGTCG | SEQ ID NO: 4 |
| MKO10014128_Dhx37 | CAAGCTCCCGATCCTCGCCG | SEQ ID NO: 5 |
| MKO10014129_Dhx37 | CTTGCTCCTCGGCGAGGATC | SEQ ID NO: 6 |
| MKO10014130_Dhx37 | TCATCTCGGCCTCCGATACT | SEQ ID NO: 7 |
| MKO10081526_Dhx37 | TTCACGGGGATGAATACAGC | SEQ ID NO: 8 |
| MKO10081527_Dhx37 | GCTTCCGGTGGGCCCCGCTG | SEQ ID NO: 9 |
| MKO10081528_Dhx37 | ACTGAGTGAAGTCCAAGTAT | SEQ ID NO: 10 |

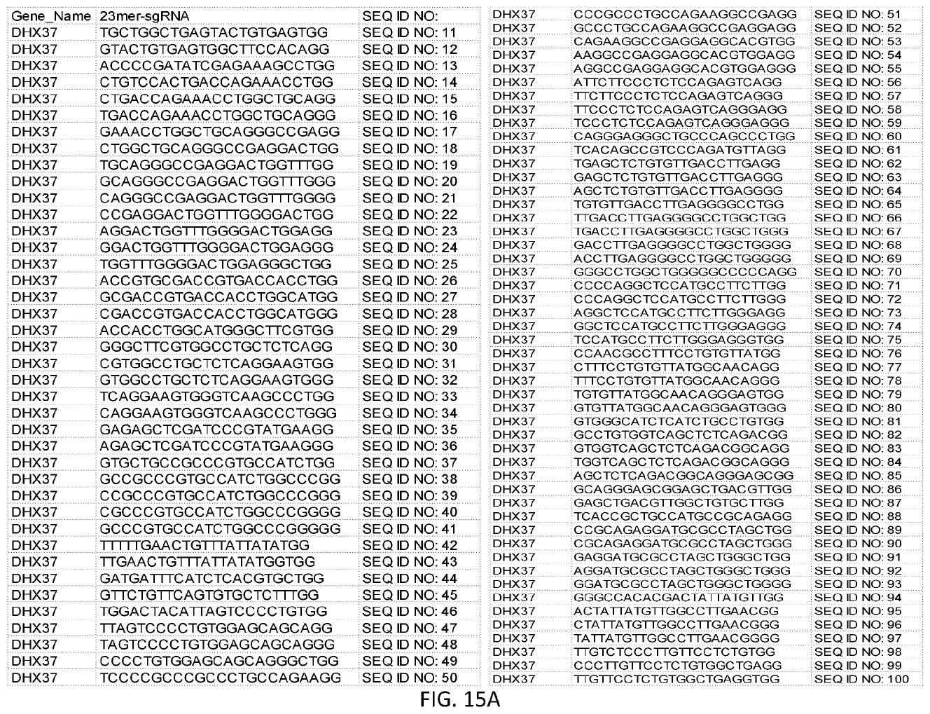

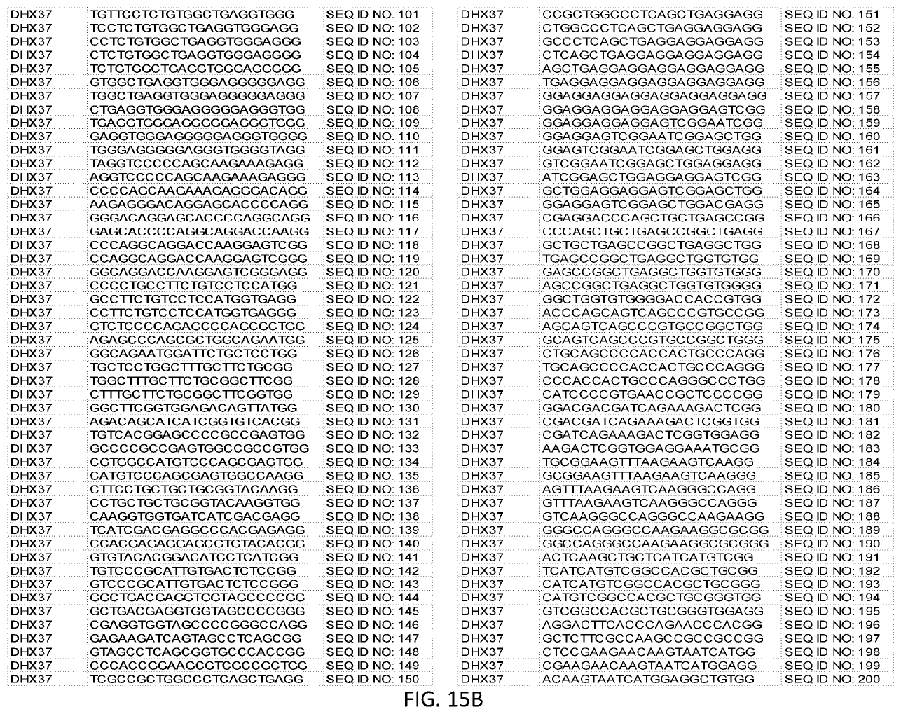

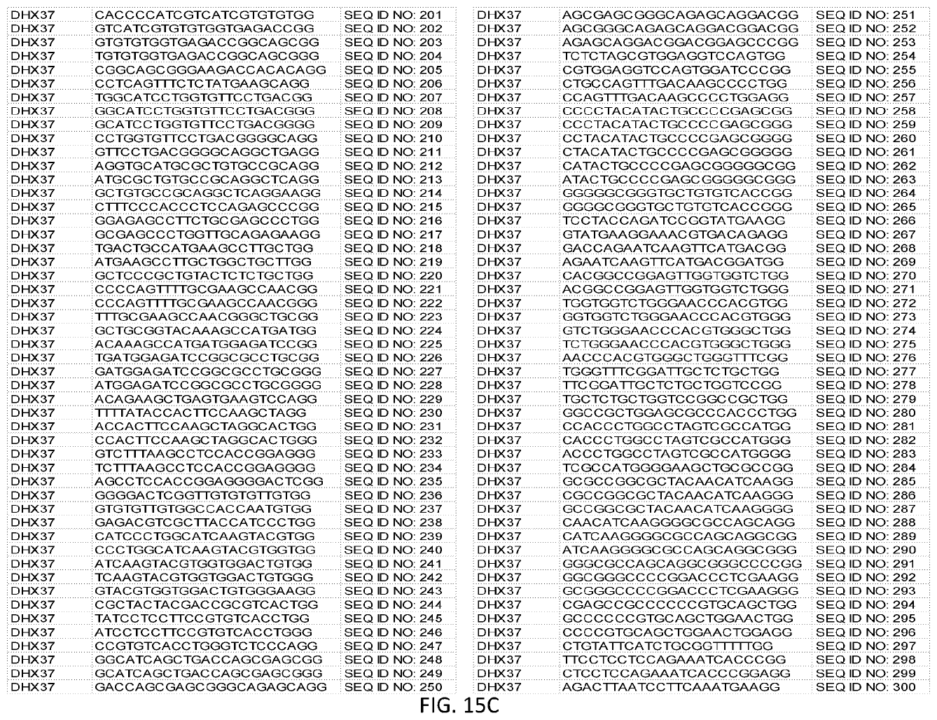

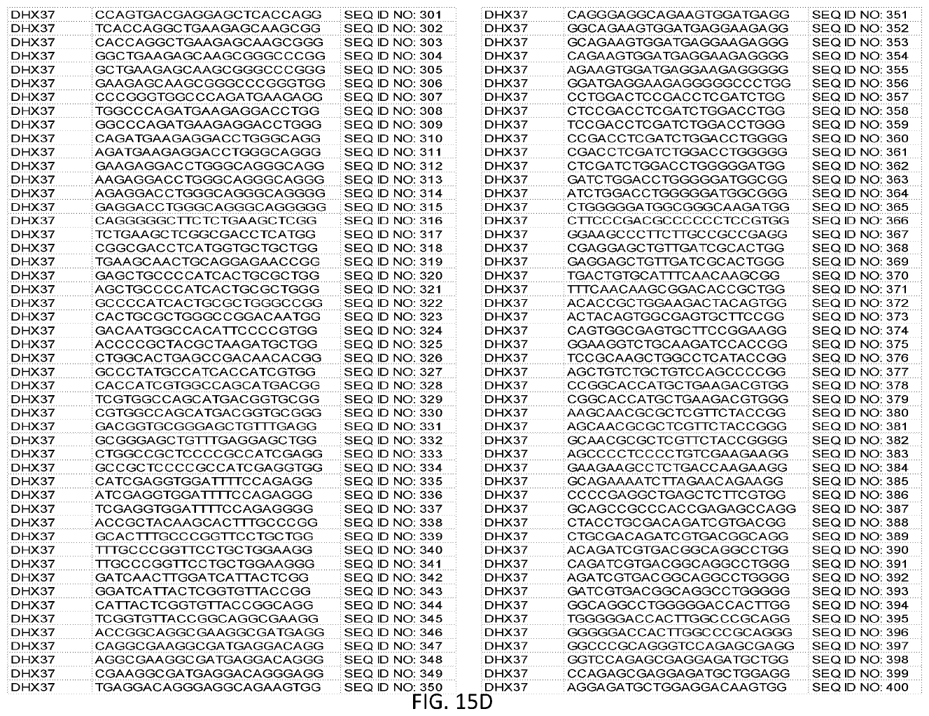

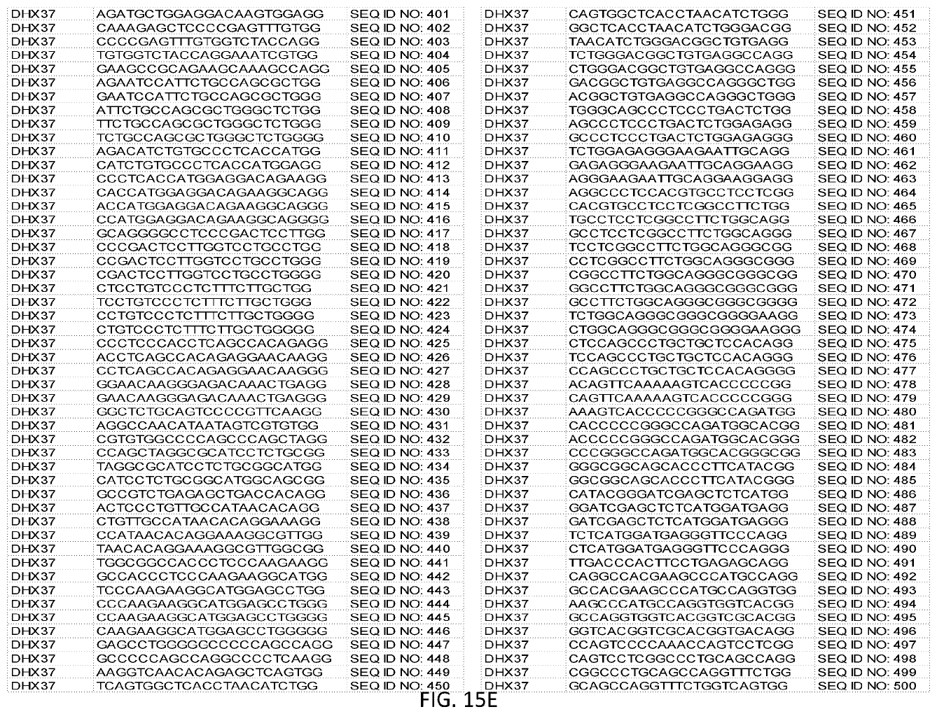

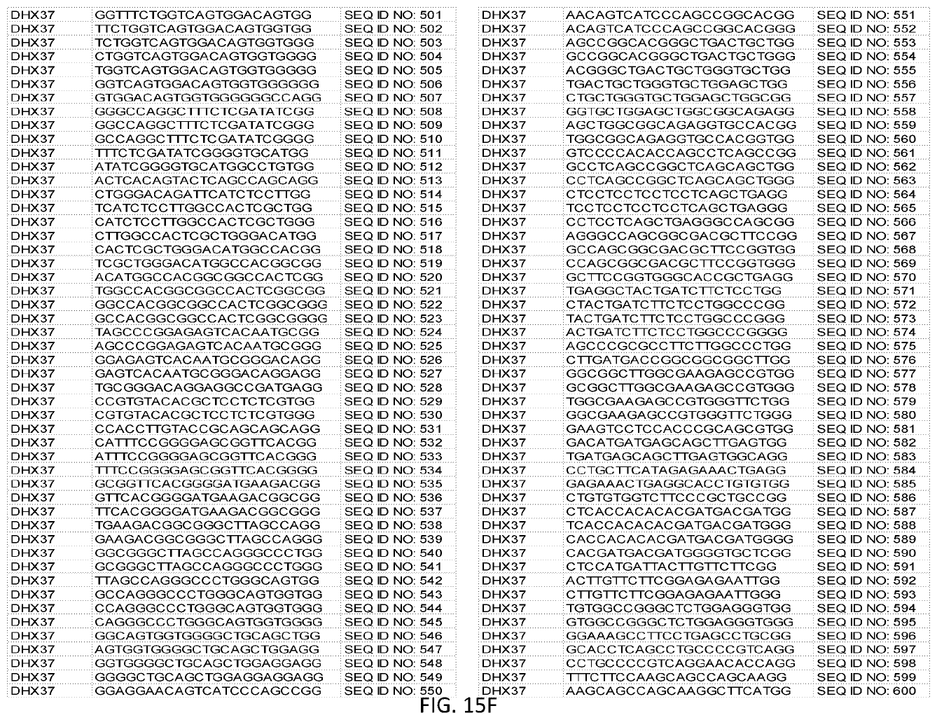

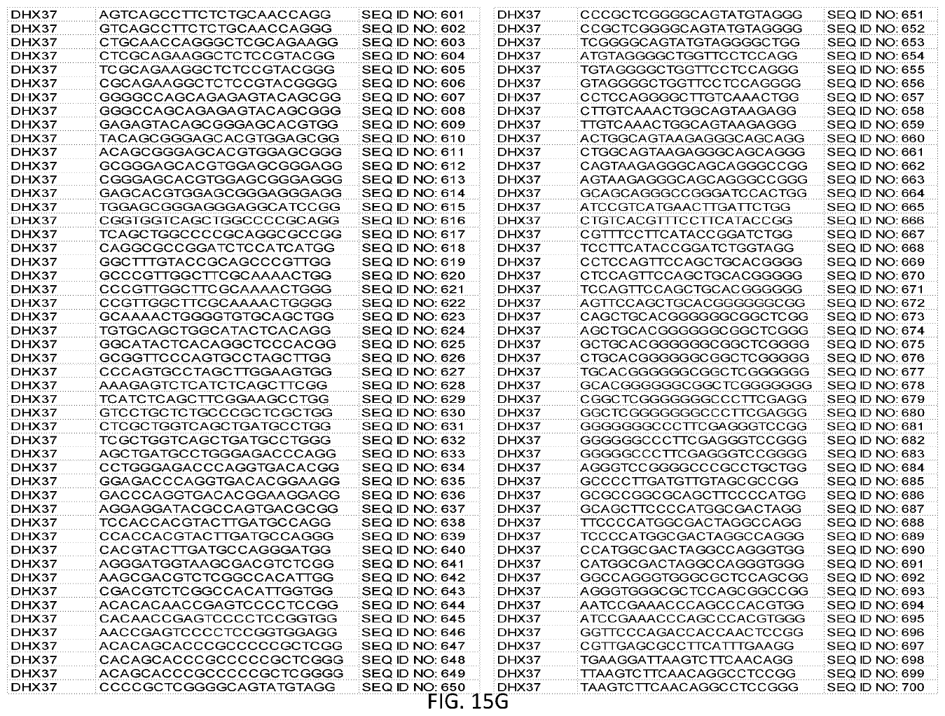

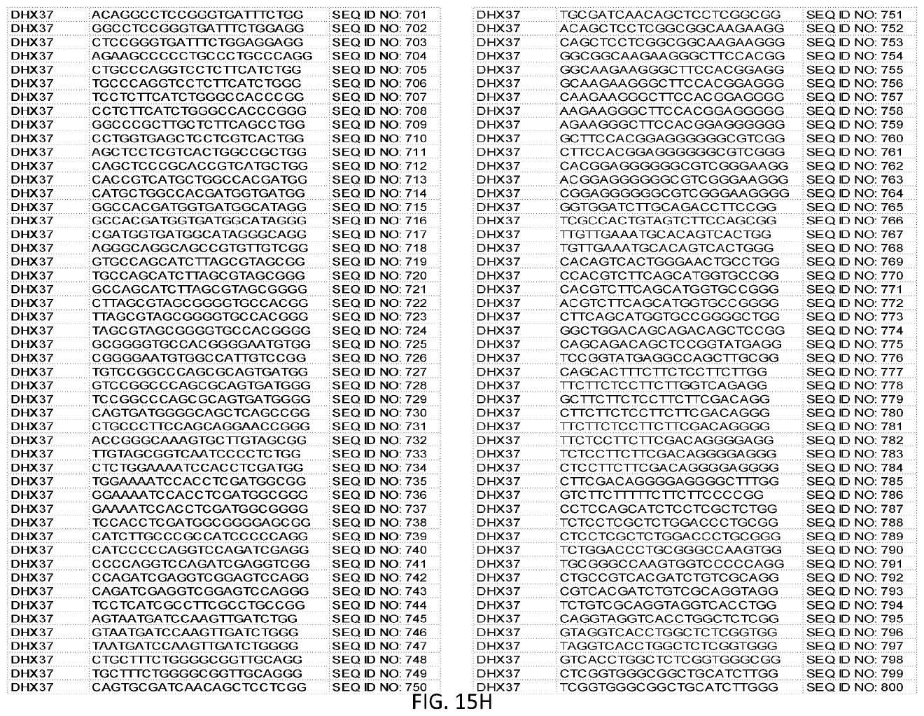

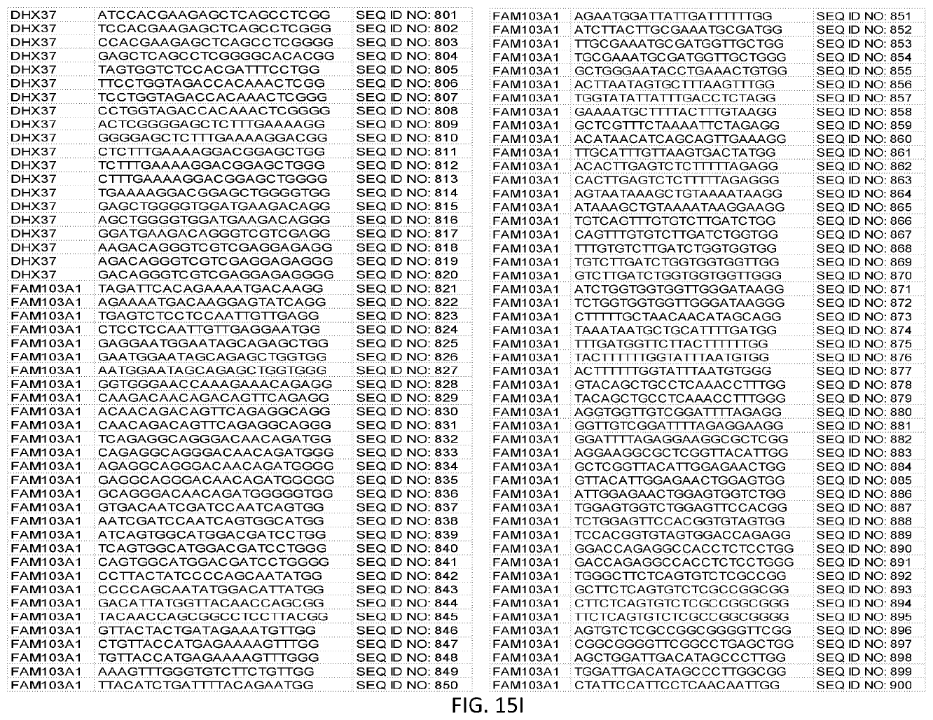

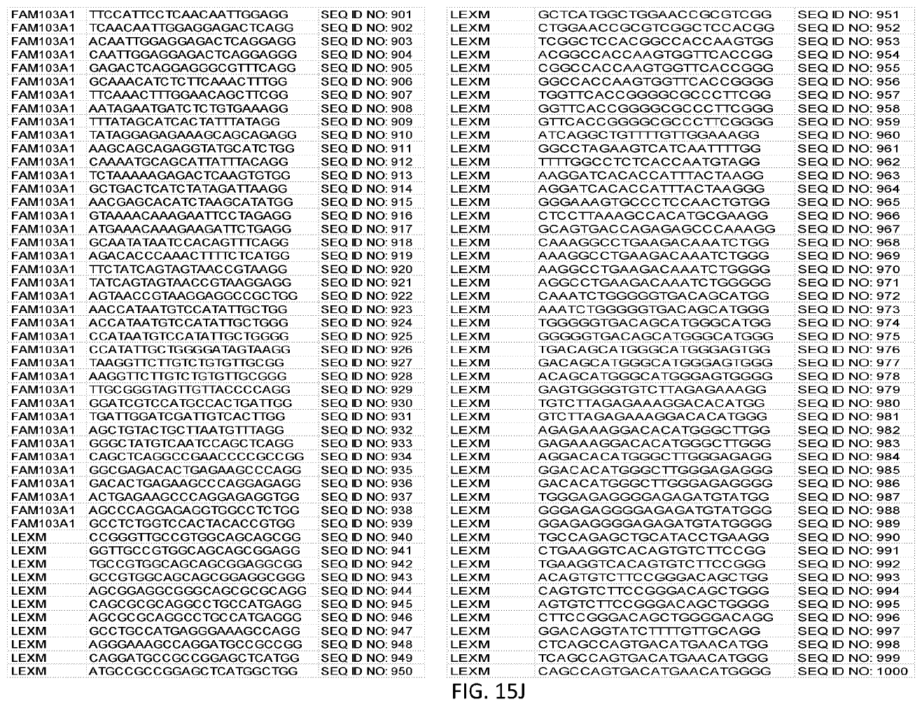

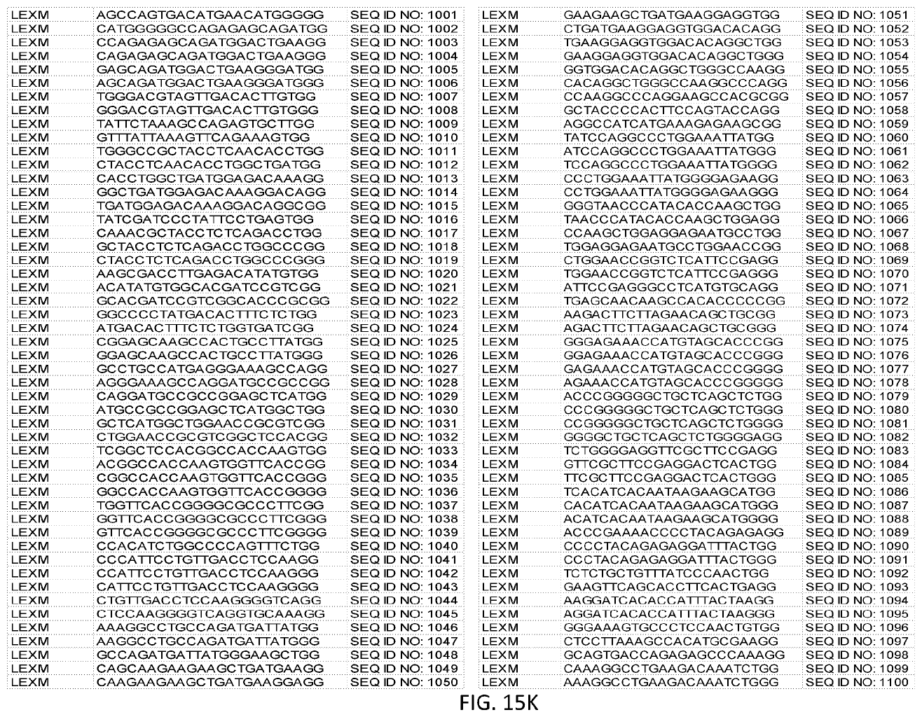

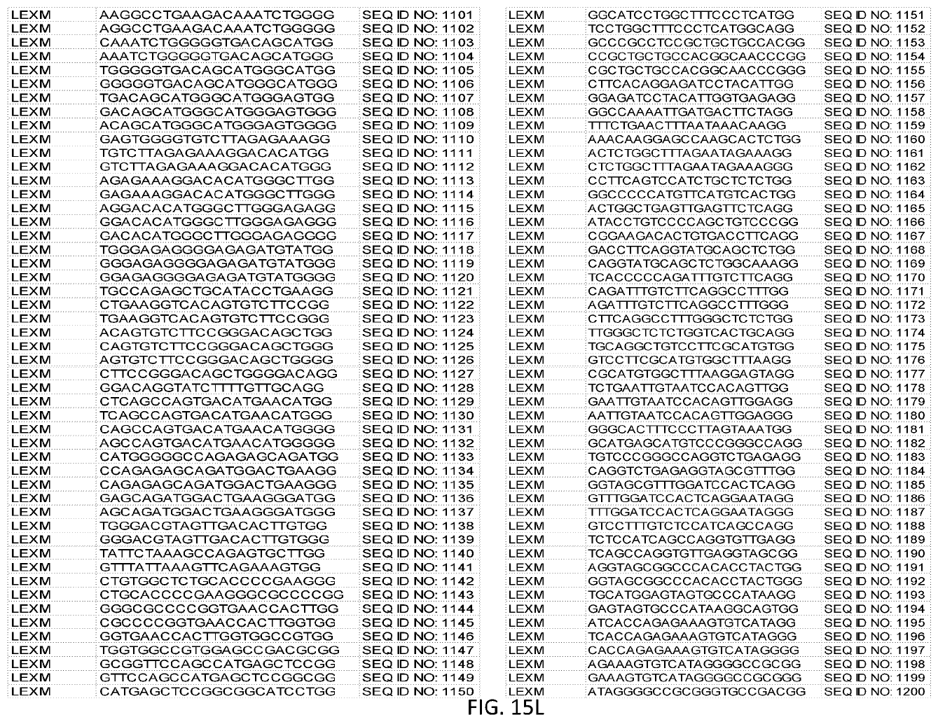

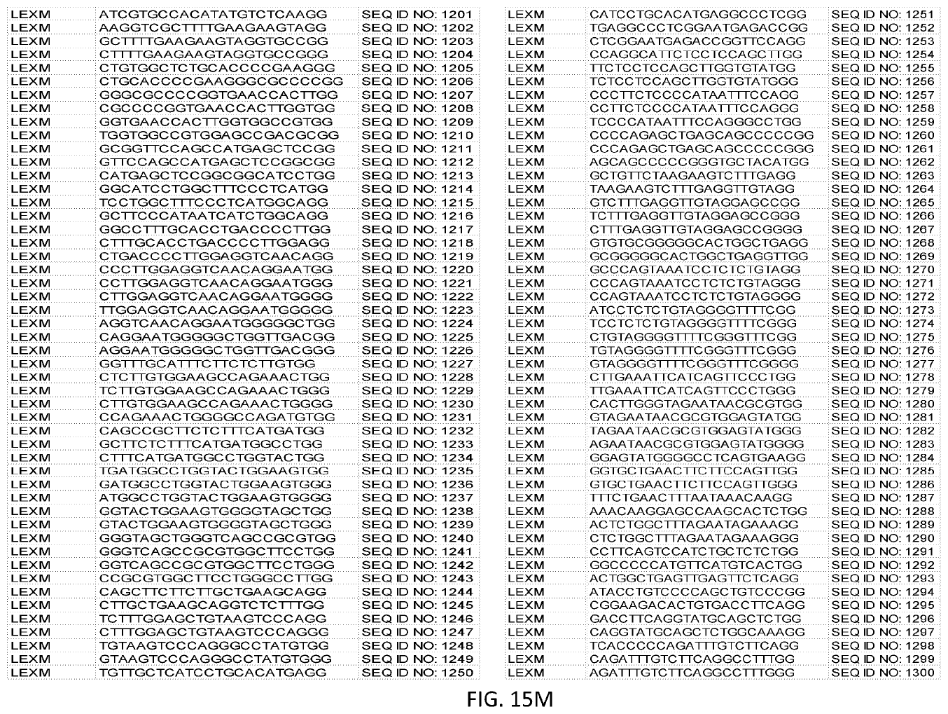

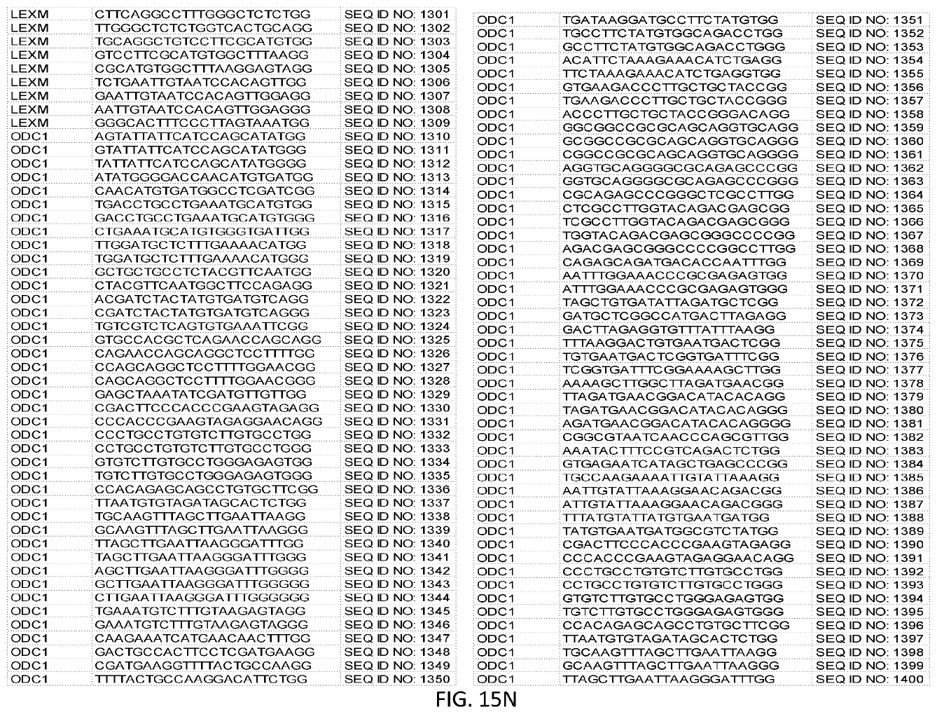

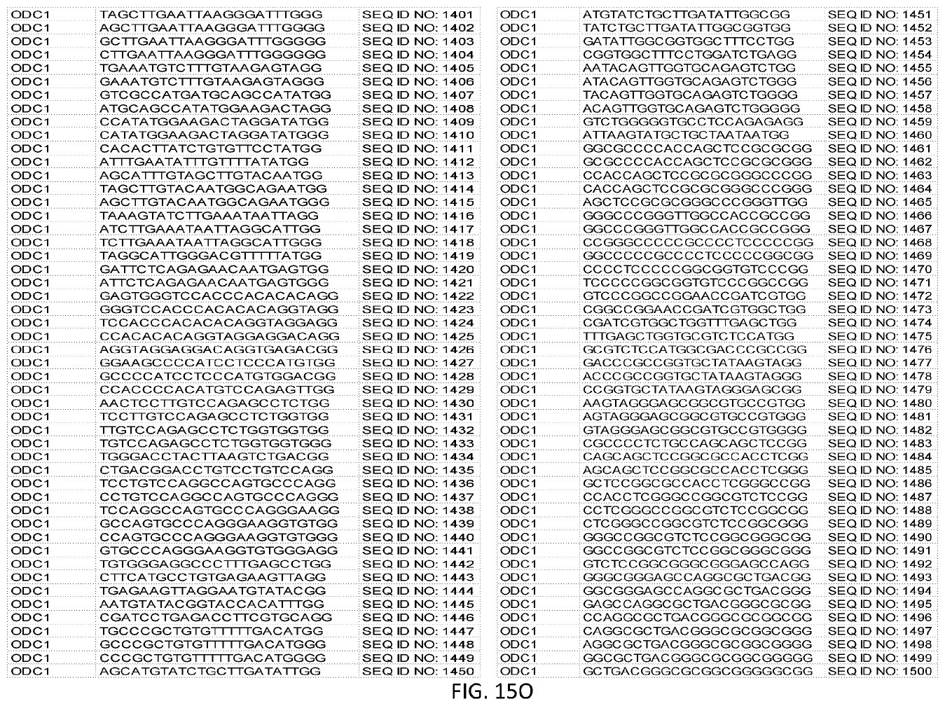

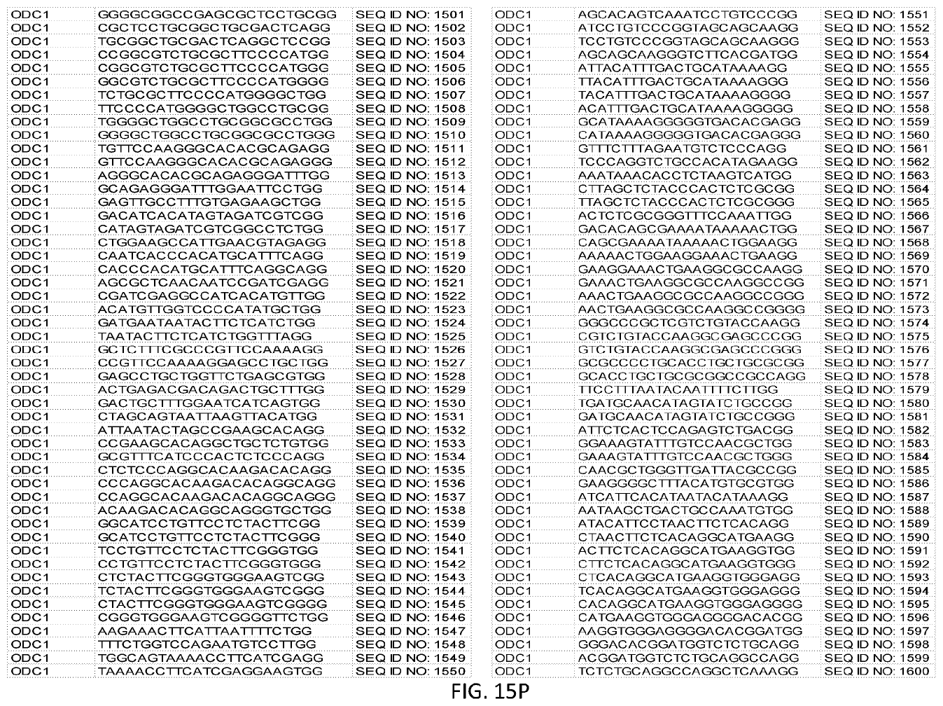

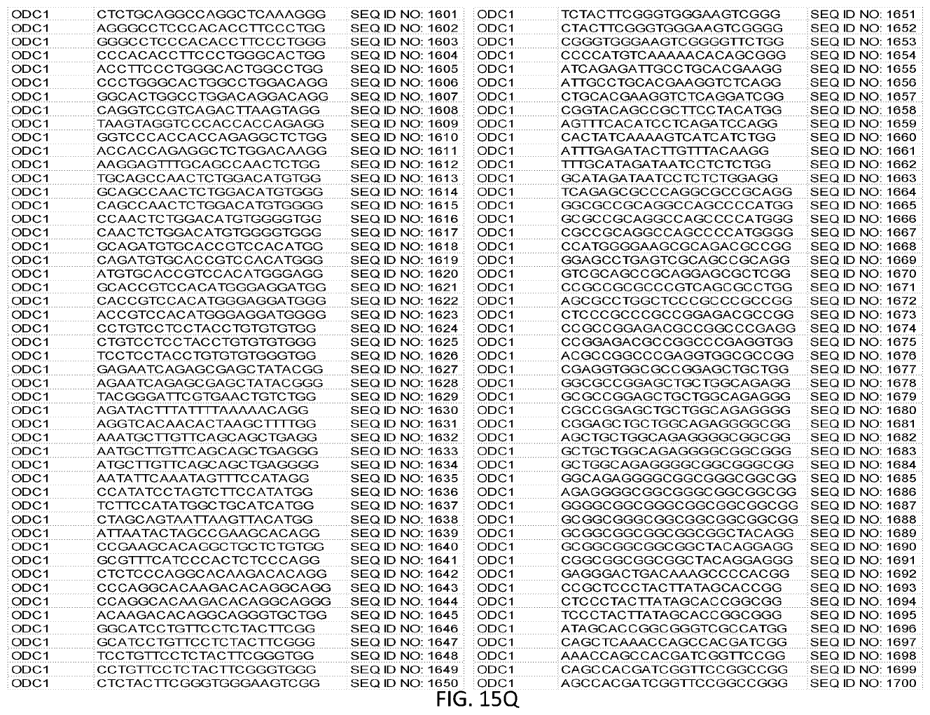

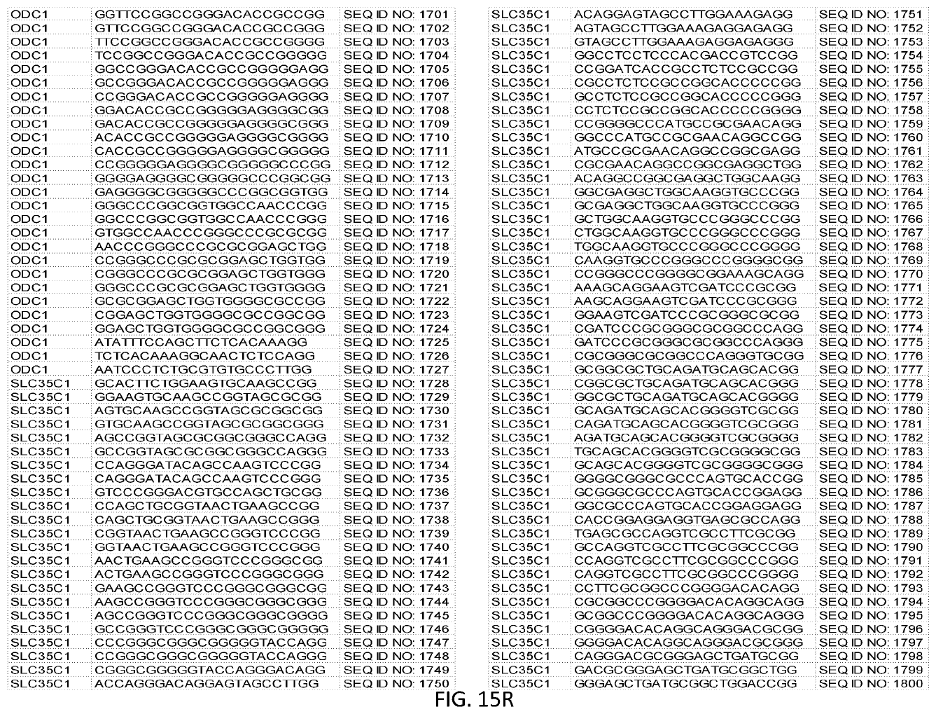

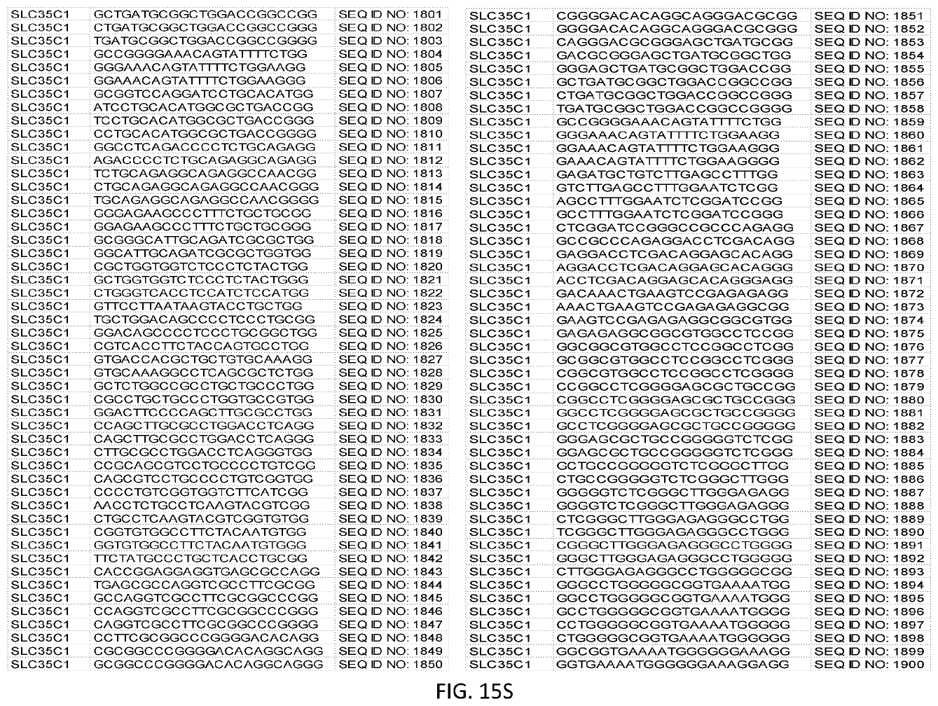

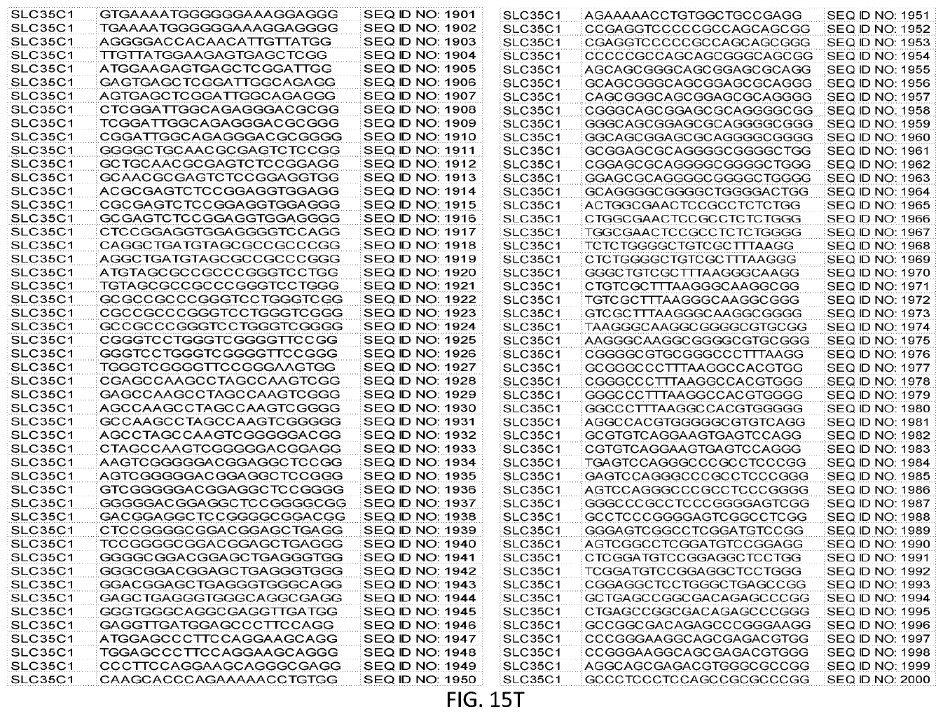

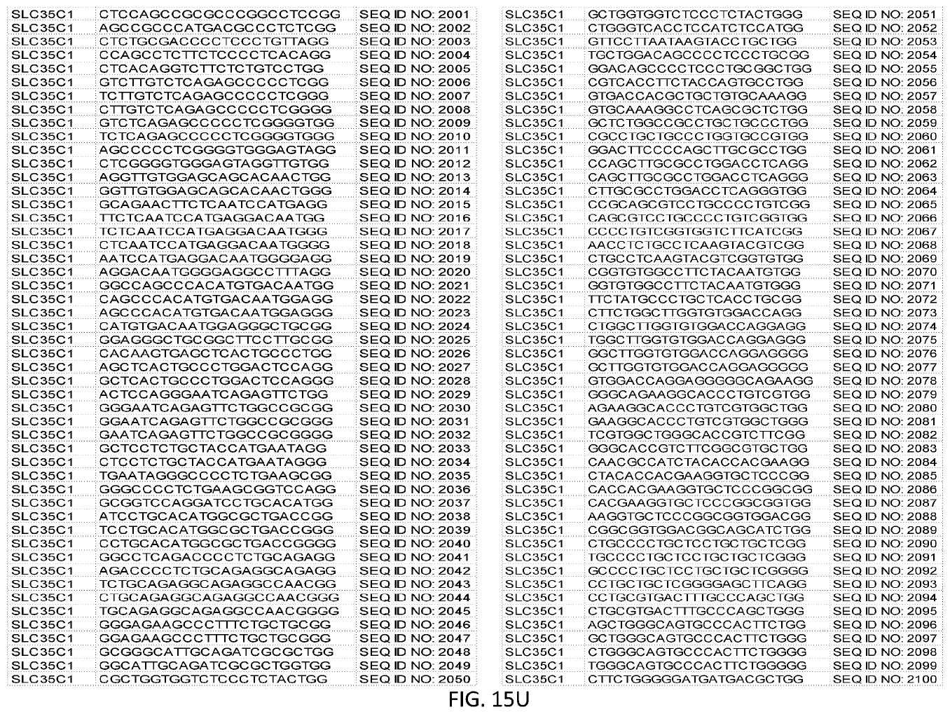

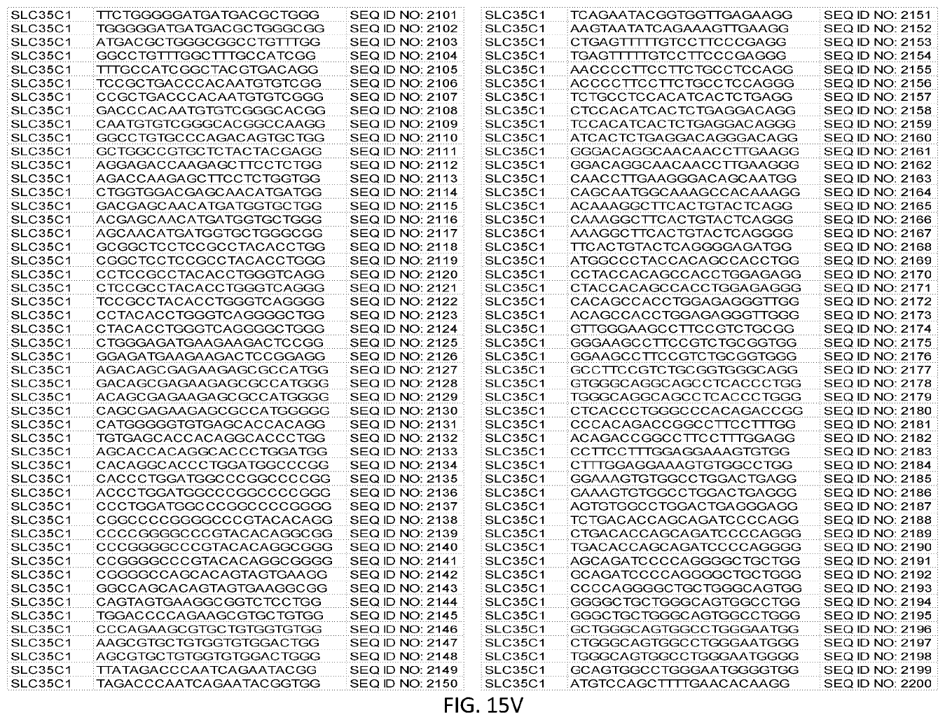

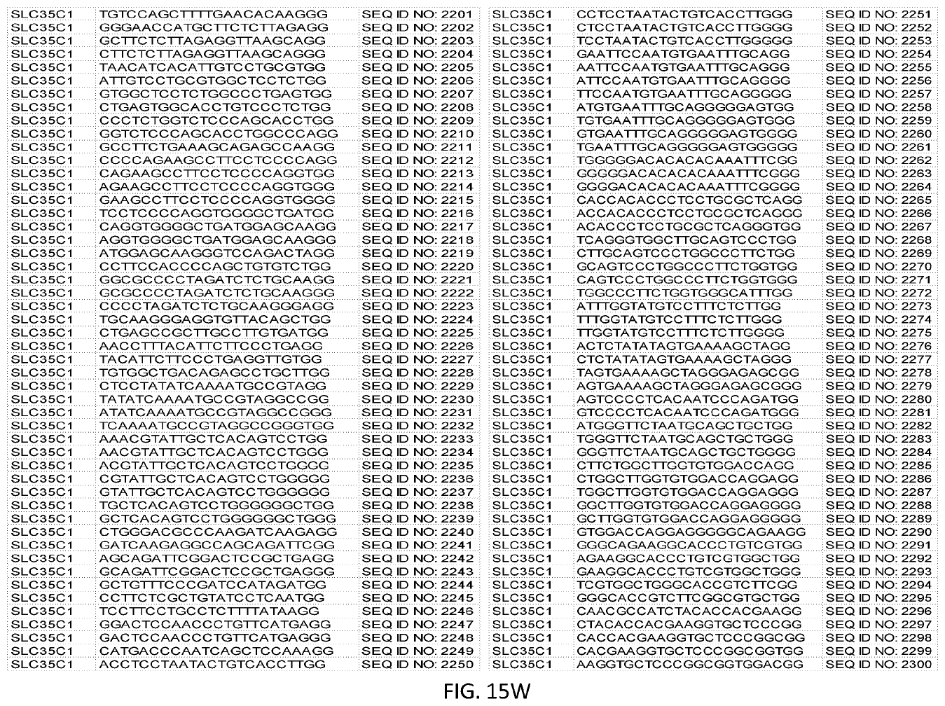

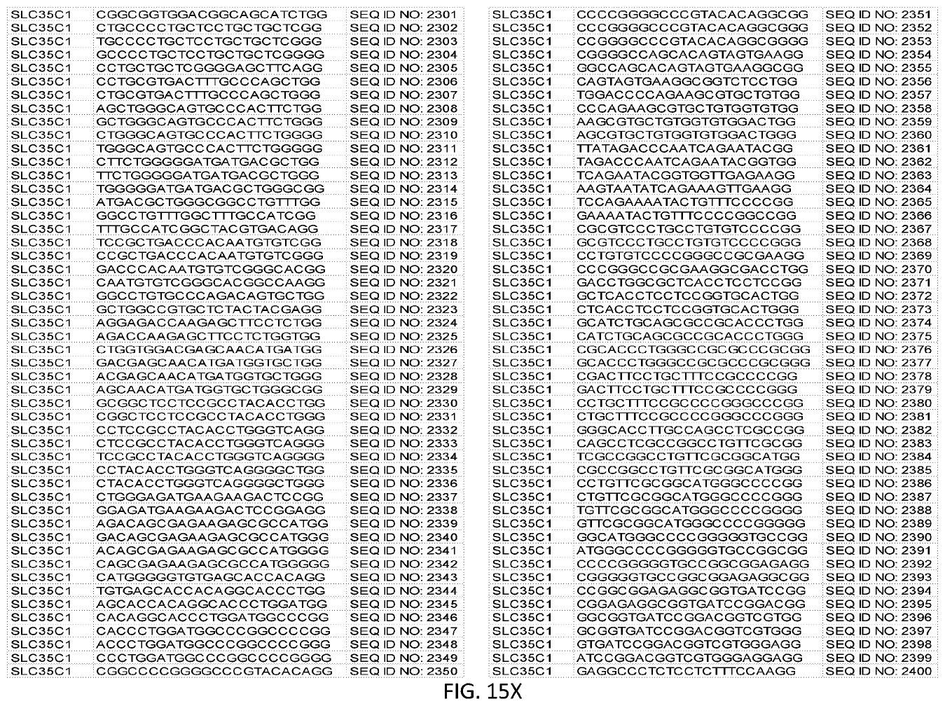

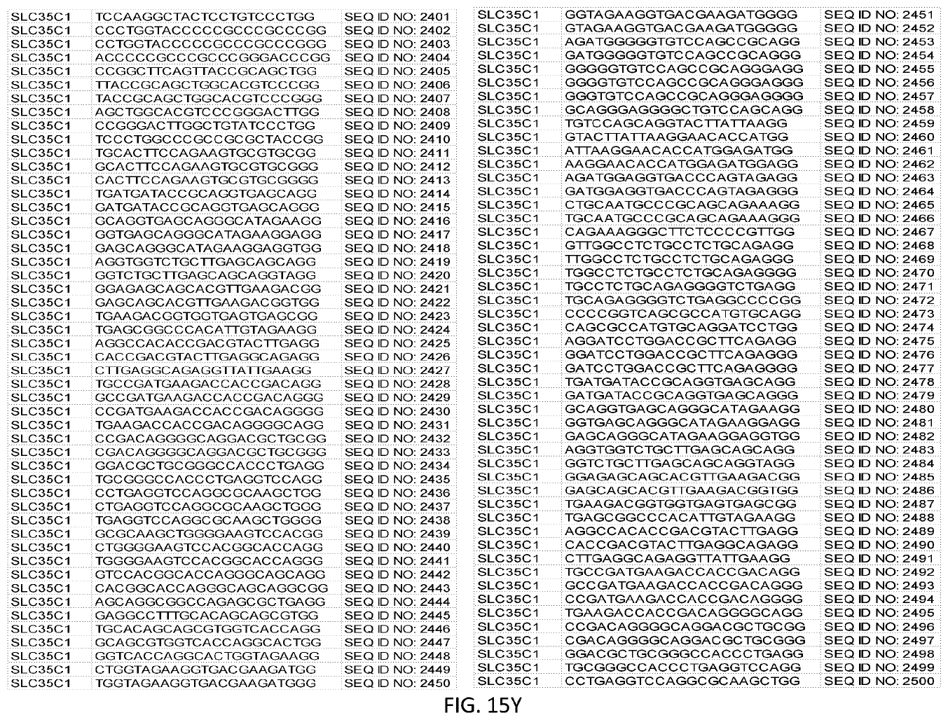

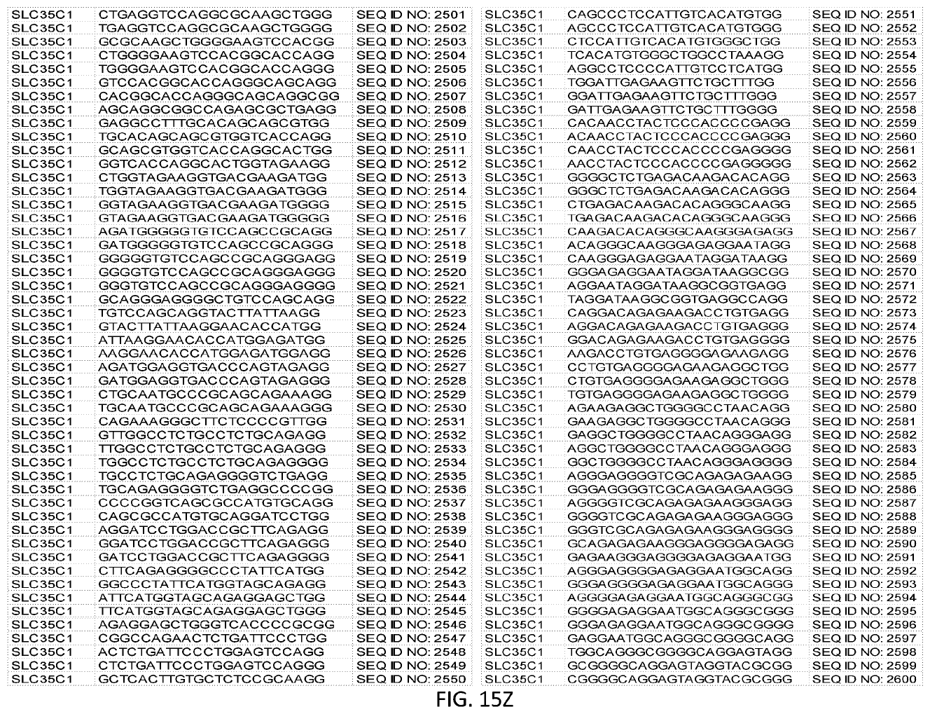

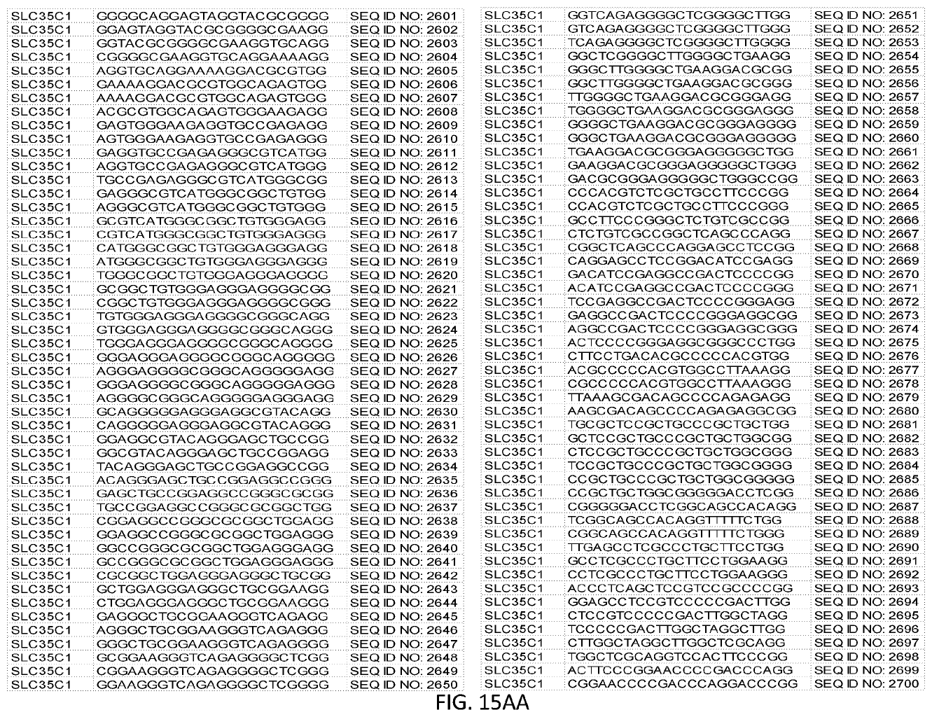

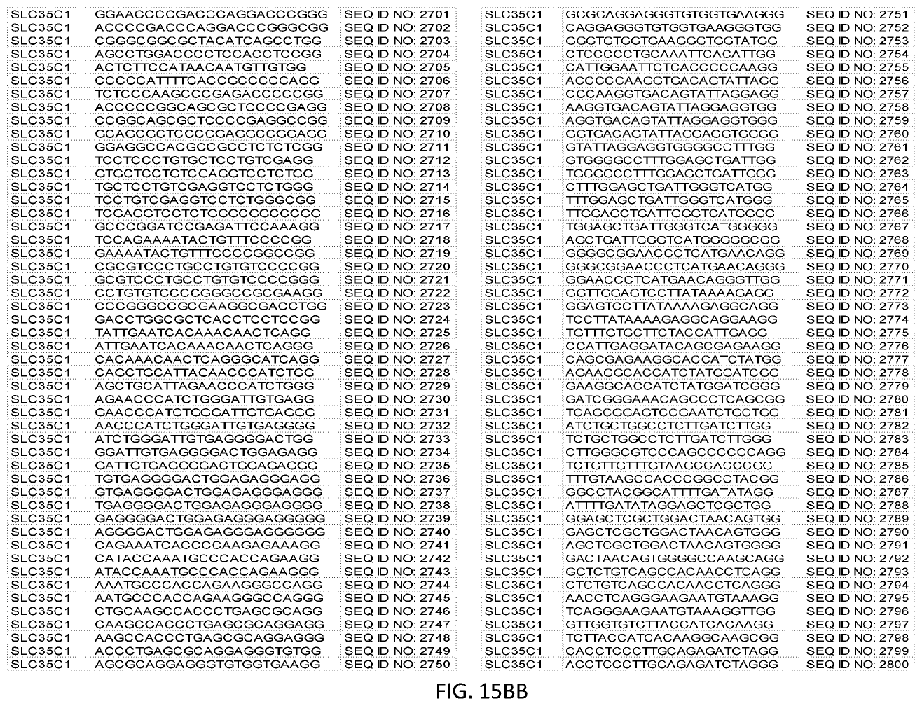

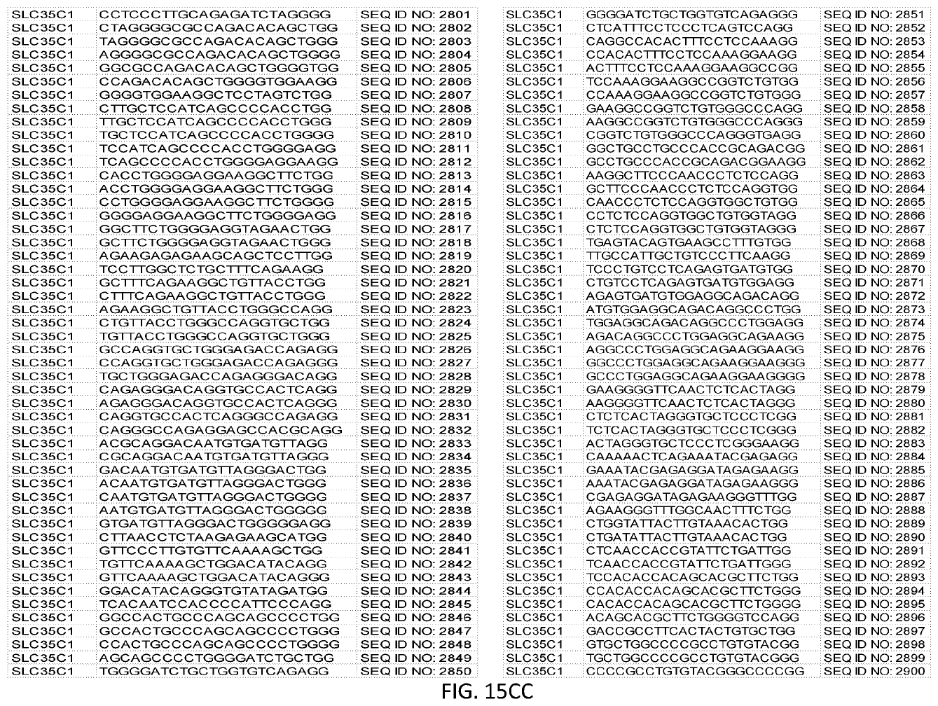

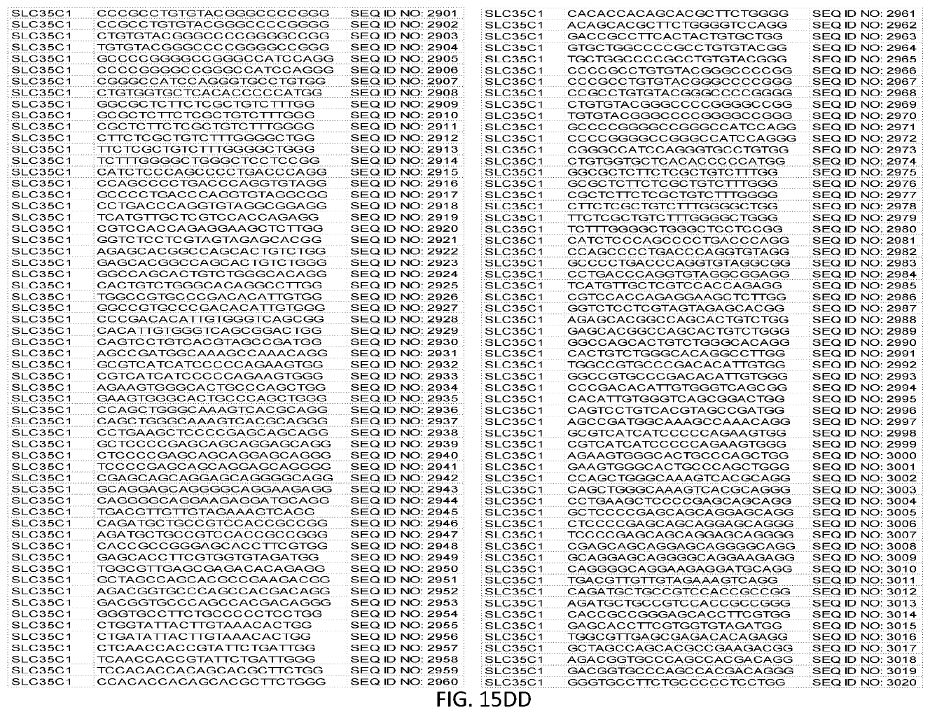

In another aspect, the invention provides a plurality of sgRNAs targeting human genes of the top hits identified in the T cell screens described herein (FIGS. 15A-15DD). sgRNAs were designed to target human genes including, but not limited to, DHX37, LEXM, FAM103A1, ODC1, and SLC35C1 (SEQ ID NOs: 11-3020).

In yet another aspect of the invention, antibodies are used to inhibit Dhx37. The antibodies used recognize and bind to at least one epitope listed in Table 2 (SEQ ID NOs: 3022-3031).

>DHX37
(SEQ ID NO: 3021)
MGKLRRRYNIKGRQQAGPGPSKGPPEPPPVQLELEDKDTLKGVDASNALV
LPGKKKKKTKAPPLSKKEKKPLTKKEKKVLQKILEQKEKKSQRAEMLQKL
SEVQASEAEMRLFYTTSKLGTGNRMYHTKEKADEVVAPGQEKISSLSGAH
RKRRRWPSAEEEEEEEEESESELEEESELDEDPAAEPAEAGVGTTVAPLP
PAPAPSSQPVPAGMTVPPPPAAAPPLPRALAKPAVFIPVNRSPEMQEERL
KLPILSEEQVIMEAVAEHPIVIVCGETGSGKTTQVPQFLYEAGFSSEDSI
IGVTEPRRVAAVAMSQRVAKEMNLSQRVVSYQIRYEGNVTEETRIKFMTD
GVLLKEIQKDFLLLRYKVVIIDEAHERSVYTDILIGLLSRIVTLRAKRNL
PLKLLIMSATLRVEDFTQNPRLFAKPPPVIKVESRQFPVTVHFNKRTPLE
DYSGECFRKVCKIHRMLPAGGILVFLTGQAEVHALCRRLRKAFPPSRARP
QEKDDDQKDSVEEMRKFKKSRARAKKARAEVLPQINLDHYSVLPAGEGDE
DREAEVDEEEGALDSDLDLDLGDGGQDGGEQPDASLPLHVLPLYSLLAPE
KQAQVFKPPPEGTRLCVVATNVAETSLTIPGIKYVVDCGKVKKRYYDRVT
GVSSFRVTWVSQASADQRAGRAGRTEPGHCYRLYSSAVFGDFEQFPPPEI
TRRPVEDLILQMKALNVEKVINFPFPTPPSVEALLAAEELLIALGALQPP
QKAERVKQLQENRLSCPITALGRTMATFPVAPRYAKMLALSRQHGCLPYA
ITIVASMTVRELFEELDRPAASDEELTRLKSKRARVAQMKRTWAGQGASL
KLGDLMVLLGAVGACEYASCTPQFCEANGLRYKAMMEIRRLRGQLTTAVN
AVCPEAELFVDPKMQPPTESQVTYLRQIVTAGLGDHLARRVQSEEMLEDK
WRNAYKTPLLDDPVFIHPSSVLFKELPEFVVYQEIVETTKMYMKGVSSVE
VQWIPALLPSYCQFDKPLEEPAPTYCPERGRVLCHRASVFYRVGWPLPAI
EVDFPEGIDRYKHFARFLLEGQVFRKLASYRSCLLSSPGTMLKTWARLQP
RTESLLRALVAEKADCHEALLAAWKKNPKYLLAEYCEWLPQAMHPDIEKA
WPPTTVH

TABLE 2
Epitopes recognized by anti-DHX37 Antibodies

| Rank | Location | Epitope | Score | SEQ ID NO: |
|---|---|---|---|---|
| 1 | 873-892 | QFCEANGLRYKAMMEIRRLR | 1.000 | (SEQ ID NO: 3022) |
| 2 | 623-642 | AETSLTIPGIKYVVDCGKVK | 0.786 | (SEQ ID NO: 3023) |
| 3 | 363-382 | LLRYKVVIIDEAHERSVYTD | 0.749 | (SEQ ID NO: 3024) |
| 4 | 34-53 | LEDKDTLKGVDASNALVLPG | 0.591 | (SEQ ID NO: 3025) |
| 5 | 260-279 | VIMEAVAEHPIVIVCGETGS | 0.532 | (SEQ ID NO: 3026) |
| 6 | 931-950 | AGLGDHLARRVQSEEMLEDK | 0.406 | (SEQ ID NO: 3027) |
| 7 | 309-328 | VAAVAMSQRVAKEMNLSQRV | 0.397 | (SEQ ID NO: 3028) |
| 8 | 226-245 | LPRALAKPAVFIPVNRSPEM | 0.393 | (SEQ ID NO: 3029) |
| 9 | 140-159 | QEKISSLSGAHRKRRRWPSA | 0.386 | (SEQ ID NO: 3030) |
| 10 | 989-1008 | TKMYMKGVSSVEVQWIPALL | 0.385 | (SEQ ID NO: 3031) |

In yet another aspect, the invention provides a kit comprising an inhibitor of Dhx37, wherein the inhibitor is selected from the group consisting of an antibody, an siRNA, and a CRISPR system. In one embodiment, the CRISPR system comprises a Cas9, and at least one sgRNA complementary to Dhx37. In another embodiment, the sgRNA comprises a nucleotide sequence selected from the group consisting of SEQ ID NOs: 1-10. In another embodiment, the sgRNA comprises a nucleotide sequence selected from the group consisting of SEQ ID NOs: 11-820. In yet another embodiment, the antibody recognizes and binds to at least one epitope sequence selected from the group consisting of SEQ ID NOs: 3022-3031.

In still another aspect, the invention includes a kit comprising a plurality of sgRNAs comprising the nucleotide sequences selected from the group consisting of SEQ ID NOs: 11-3020.

Instructional material for use thereof is also included with the kits. Instructional material can include directions for using the components of the kit as well as instructions or guidance for interpreting the results.

### Methods

In one aspect, the invention includes a method of enhancing T cell based immunotherapy. Another aspect includes a method of performing adoptive cell transfer. Yet another aspect includes a method of treating cancer in a subject. In certain embodiments, the method comprises administering to a subject in need thereof a genetically modified T cell wherein a gene selected from the group consisting of Dhx37, Lyn, Slc35c1, Lexm, Fam103a1 and Odc1 has been mutated in the T cell. In certain embodiments, the method comprises administering to a subject in need thereof a genetically modified T cell wherein the Dhx37 gene has been mutated in the T cell. The T cell can be any subset of T cells, including but not limited to CD8+, CD4+, T regulatory (Treg) cells, and CAR T-cells. In certain embodiments, additional genes are mutated in the T cell. The additional mutated genes can include, but are not limited to, Dhx37, Lyn, Slc35c1, Lexm, Fam103a1 and Odc1.

Another aspect of the invention includes a method of treating cancer in subject in need thereof comprising administering to the subject a therapeutically effective amount of an inhibitor of Dhx37. The inhibitor can include but is not limited to an antibody, an siRNA, and a CRISPR system. The CRISPR system can comprise a Cas9, and at least one sgRNA complementary to Dhx37 and the sgRNAs can comprise SEQ ID NOs: 1-10. In another embodiment, the sgRNAs are selected from the group consisting of SEQ ID NOs: 11-820. In another embodiment, the antibody recognizes and binds to at least one epitope sequence selected from the group consisting of SEQ ID NOs: 3022-3031.

Yet another aspect of the invention includes a method of treating cancer in subject in need thereof comprising administering to the subject a therapeutically effective amount of an inhibitor of a gene selected from the group consisting of Lyn, Slc35c1, Lexm, Fam103a1 and Odc. The inhibitor can include but is not limited to an antibody, an siRNA, and a CRISPR system. The CRISPR system can comprise a Cas9, and at least one sgRNA complementary to a gene selected from the group consisting of Lyn, Slc35c1, Lexm, Fam103a1 and Odc. In one embodiment, the sgRNA comprises a nucleotide sequence selected from the group consisting of SEQ ID NOs: 821-3020. Certain embodiments of the methods described herein include administering an additional treatment to the subject. The additional treatment can include immune checkpoint inhibitors, including but not limited to inhibitors of CTLA-4, PD-1, 4-1BB, CD27, CD28, ICOS, LAG3, OX-40, TIM3, and VISTA.

Another aspect of the invention includes a method of generating a genetically modified T cell for use in immunotherapy. In one embodiment, the method comprises administering to a naïve T cell a vector comprising a first sgRNA complementary to a first nucleotide sequence of the Dhx37 gene and a second sgRNAs complementary to a second nucleotide sequence of the Dhx37 gene. In one embodiment, the method comprises administering to a naïve T cell a vector comprising a first sgRNA complementary to a first nucleotide sequence of a gene selected from the group consisting of Lyn, Slc35c1, Lexm, Fam103a1 and Odc and a second sgRNA complementary to a second nucleotide sequence of a gene selected from the group consisting of Lyn, Slc35c1, Lexm, Fam103a1 and Odc. In one embodiment, the first sgRNA is selected from the group consisting of SEQ ID NOs: 1-10 and the second sgRNA is selected from the group consisting of SEQ ID NOs: 1-10. In another embodiment, the first sgRNA is selected from the group consisting of SEQ ID NOs: 11-820 and the second sgRNA is selected from the group consisting of SEQ ID NOs: 11-820. In one embodiment, the first sgRNA nucleotide sequence is selected from the group consisting of SEQ ID NOs: 821-3020 and the second sgRNA nucleotide sequence is selected from the group consisting of SEQ ID NOs: 821-3020.

The mutations introduced by the methods described herein can be any combination of insertions or deletions, including but not limited to a single base insertion, a single base deletion, a frameshift, a rearrangement, and an insertion or deletion of 2, 3, 4, 5, 6, 7, 8, 9, 10, 15, 20, 25, 30, 40, 50, 60, 70, 80, 90, 100, 150, 200, 250, 300, any and all numbers in between, bases. The mutation can occur in a gene or in a non-coding region.

In certain embodiments of the invention, the subject is a human. Other subjects that can be used include but are not limited to mice, rats, rabbits, dogs, cats, horses, pigs, cows and birds. The compositions of the invention can be administered to an animal by any means standard in the art. For example the vectors can be injected into the animal. The injections can be intravenous, subcutaneous, intraperitoneal, or directly into a tissue or organ. In certain embodiments, the genetically modified T cells of the invention are adoptively transferred to the animal.

### CRISPR/Cas9

The CRISPR/Cas9 system is a facile and efficient system for inducing targeted genetic alterations. Target recognition by the Cas9 protein requires a ‘seed’ sequence within the guide RNA (gRNA) and a conserved dinucleotide containing protospacer adjacent motif (PAI) sequence upstream of the gRNA-binding region. The CRISPR/Cas9 system can thereby be engineered to cleave virtually any DNA sequence by redesigning the gRNA in cell lines (such as 293T cells), primary cells, and CAR T cells. The CRISPR/Cas9 system can simultaneously target multiple genomic loci by co-expressing a single Cas9 protein with two or more gRNAs, making this system uniquely suited for multiple gene editing or synergistic activation of target genes.

The Cas9 protein and guide RNA form a complex that identifies and cleaves target sequences. Cas9 is comprised of six domains: REC I, REC II, Bridge Helix, PAM interacting, HNH, and RuvC. The RecI domain binds the guide RNA, while the Bridge helix binds to target DNA. The HNH and RuvC domains are nuclease domains. Guide RNA is engineered to have a 5′ end that is complementary to the target DNA sequence. Upon binding of the guide RNA to the Cas9 protein, a conformational change occurs activating the protein.

Once activated, Cas9 searches for target DNA by binding to sequences that match its protospacer adjacent motif (PAM) sequence. A PAM is a two or three nucleotide base sequence within one nucleotide downstream of the region complementary to the guide RNA.

In one non-limiting example, the PAM sequence is 5′-NGG-3′. When the Cas9 protein finds its target sequence with the appropriate PAM, it melts the bases upstream of the PAI and pairs them with the complementary region on the guide RNA. Then the RuvC and HNH nuclease domains cut the target DNA after the third nucleotide base upstream of the PAM.

One non-limiting example of a CRISPR/Cas system used to inhibit gene expression, CRISPRi, is described in U.S. Patent Appl. Publ. No. US20140068797. CRISPRi induces permanent gene disruption that utilizes the RNA-guided Cas9 endonuclease to introduce DNA double stranded breaks, which trigger error-prone repair pathways to result in frame shift mutations. A catalytically dead Cas9 lacks endonuclease activity. When coexpressed with a guide RNA, a DNA recognition complex is generated that specifically interferes with transcriptional elongation, RNA polymerase binding, or transcription factor binding. This CRISPRi system efficiently represses expression of targeted genes.

CRISPR/Cas gene disruption occurs when a guide nucleotide sequence specific for a target gene and a Cas endonuclease are introduced into a cell and form a complex that enables the Cas endonuclease to introduce a double strand break at the target gene. In certain embodiments, the CRISPR/Cas system comprises an expression vector, such as, but not limited to, an pAd5F35-CRISPR vector. In other embodiments, the Cas expression vector induces expression of Cas9 endonuclease. Other endonucleases may also be used, including but not limited to, T7, Cas3, Cas8a, Cas8b, Cas10d, Cse1, Csy1, Csn2, Cas4, Cas10, Csm2, Cmr5, Fok1, other nucleases known in the art, and any combinations thereof.

In certain embodiments, inducing the Cas expression vector comprises exposing the cell to an agent that activates an inducible promoter in the Cas expression vector. In such embodiments, the Cas expression vector includes an inducible promoter, such as one that is inducible by exposure to an antibiotic (e.g., by tetracycline or a derivative of tetracycline, for example doxycycline). However, it should be appreciated that other inducible promoters can be used. The inducing agent can be a selective condition (e.g., exposure to an agent, for example an antibiotic) that results in induction of the inducible promoter. This results in expression of the Cas expression vector.

In certain embodiments, guide RNA(s) and Cas9 can be delivered to a cell as a ribonucleoprotein (RNP) complex. RNPs are comprised of purified Cas9 protein complexed with gRNA and are well known in the art to be efficiently delivered to multiple types of cells, including but not limited to stem cells and immune cells (Addgene, Cambridge, Mass., Mirus Bio LLC, Madison, Wis.).

The guide RNA is specific for a genomic region of interest and targets that region for Cas endonuclease-induced double strand breaks. The target sequence of the guide RNA sequence may be within a loci of a gene or within a non-coding region of the genome. In certain embodiments, the guide nucleotide sequence is at least 10, 11, 12, 13, 14, 15, 16, 17, 18, 19, 20, 21, 22, 23, 24, 25, 26, 27, 30, 31, 32, 33, 34, 35, 36, 37, 38, 39, 40 or more nucleotides in length.

Guide RNA (gRNA), also referred to as “short guide RNA” or “sgRNA”, provides both targeting specificity and scaffolding/binding ability for the Cas9 nuclease. The gRNA can be a synthetic RNA composed of a targeting sequence and scaffold sequence derived from endogenous bacterial crRNA and tracrRNA. gRNA is used to target Cas9 to a specific genomic locus in genome engineering experiments. Guide RNAs can be designed using standard tools well known in the art.

In the context of formation of a CRISPR complex, “target sequence” refers to a sequence to which a guide sequence is designed to have some complementarity, where hybridization between a target sequence and a guide sequence promotes the formation of a CRISPR complex. Full complementarity is not necessarily required, provided there is sufficient complementarity to cause hybridization and promote formation of a CRISPR complex. A target sequence may comprise any polynucleotide, such as a DNA or a RNA polynucleotide. In certain embodiments, a target sequence is located in the nucleus or cytoplasm of a cell. In other embodiments, the target sequence may be within an organelle of a eukaryotic cell, for example, mitochondrion or nucleus. Typically, in the context of an endogenous CRISPR system, formation of a CRISPR complex (comprising a guide sequence hybridized to a target sequence and complexed with one or more Cas proteins) results in cleavage of one or both strands in or near (e.g., within about 1, 2, 3, 4, 5, 6, 7, 8, 9, 10, 20, 50 or more base pairs) the target sequence. As with the target sequence, it is believed that complete complementarity is not needed, provided this is sufficient to be functional.

In certain embodiments, one or more vectors driving expression of one or more elements of a CRISPR system are introduced into a host cell, such that expression of the elements of the CRISPR system direct formation of a CRISPR complex at one or more target sites. For example, a Cas enzyme, a guide sequence linked to a tracr-mate sequence, and a tracr sequence could each be operably linked to separate regulatory elements on separate vectors. Alternatively, two or more of the elements expressed from the same or different regulatory elements may be combined in a single vector, with one or more additional vectors providing any components of the CRISPR system not included in the first vector. CRISPR system elements that are combined in a single vector may be arranged in any suitable orientation, such as one element located 5′ with respect to (“upstream” of) or 3′ with respect to (“downstream” of) a second element. The coding sequence of one element may be located on the same or opposite strand of the coding sequence of a second element, and oriented in the same or opposite direction. In certain embodiments, a single promoter drives expression of a transcript encoding a CRISPR enzyme and one or more of the guide sequence, tracr mate sequence (optionally operably linked to the guide sequence), and a tracr sequence embedded within one or more intron sequences (e.g., each in a different intron, two or more in at least one intron, or all in a single intron).

In certain embodiments, the CRISPR enzyme is part of a fusion protein comprising one or more heterologous protein domains (e.g. about or more than about 1, 2, 3, 4, 5, 6, 7, 8, 9, 10, or more domains in addition to the CRISPR enzyme). A CRISPR enzyme fusion protein may comprise any additional protein sequence, and optionally a linker sequence between any two domains. Examples of protein domains that may be fused to a CRISPR enzyme include, without limitation, epitope tags, reporter gene sequences, and protein domains having one or more of the following activities: methylase activity, demethylase activity, transcription activation activity, transcription repression activity, transcription release factor activity, histone modification activity, RNA cleavage activity and nucleic acid binding activity. Additional domains that may form part of a fusion protein comprising a CRISPR enzyme are described in U.S. Patent Appl. Publ. No. US20110059502, which is incorporated herein by reference. In certain embodiments, a tagged CRISPR enzyme is used to identify the location of a target sequence.

Conventional viral and non-viral based gene transfer methods can be used to introduce nucleic acids in mammalian and non-mammalian cells or target tissues. Such methods can be used to administer nucleic acids encoding components of a CRISPR system to cells in culture, or in a host organism. Non-viral vector delivery systems include DNA plasmids, RNA (e.g., a transcript of a vector described herein), naked nucleic acid, and nucleic acid complexed with a delivery vehicle, such as a liposome. Viral vector delivery systems include DNA and RNA viruses, which have either episomal or integrated genomes after delivery to the cell (Anderson, 1992, Science 256:808-813; and Yu, et al., 1994, Gene Therapy 1:13-26).

In certain embodiments, the CRISPR/Cas is derived from a type II CRISPR/Cas system. In some embodiments, the CRISPR/Cas system is derived from a Cas9 protein. The Cas9 protein can be from Streptococcus pyogenes, Streptococcus thermophilus, or other species.

In general, Cas proteins comprise at least one RNA recognition and/or RNA binding domain. RNA recognition and/or RNA binding domains interact with the guiding RNA. Cas proteins can also comprise nuclease domains (i.e., DNase or RNase domains), DNA binding domains, helicase domains, RNAse domains, protein-protein interaction domains, dimerization domains, as well as other domains. The Cas proteins can be modified to increase nucleic acid binding affinity and/or specificity, alter an enzymatic activity, and/or change another property of the protein. In certain embodiments, the Cas-like protein of the fusion protein can be derived from a wild type Cas9 protein or fragment thereof. In other embodiments, the Cas can be derived from modified Cas9 protein. For example, the amino acid sequence of the Cas9 protein can be modified to alter one or more properties (e.g., nuclease activity, affinity, stability, and so forth) of the protein. Alternatively, domains of the Cas9 protein not involved in RNA-guided cleavage can be eliminated from the protein such that the modified Cas9 protein is smaller than the wild type Cas9 protein. In general, a Cas9 protein comprises at least two nuclease (i.e., DNase) domains. For example, a Cas9 protein can comprise a RuvC-like nuclease domain and a HNH-like nuclease domain. The RuvC and HNH domains work together to cut single strands to make a double-stranded break in DNA. (Jinek, et al., 2012, Science, 337:816-821). In certain embodiments, the Cas9-derived protein can be modified to contain only one functional nuclease domain (either a RuvC-like or a HNH-like nuclease domain). For example, the Cas9-derived protein can be modified such that one of the nuclease domains is deleted or mutated such that it is no longer functional (i.e., the nuclease activity is absent). In some embodiments in which one of the nuclease domains is inactive, the Cas9-derived protein is able to introduce a nick into a double-stranded nucleic acid (such protein is termed a “nickase”), but not cleave the double-stranded DNA. In any of the above-described embodiments, any or all of the nuclease domains can be inactivated by one or more deletion mutations, insertion mutations, and/or substitution mutations using well-known methods, such as site-directed mutagenesis, PCR-mediated mutagenesis, and total gene synthesis, as well as other methods known in the art.

In one non-limiting embodiment, a vector drives the expression of the CRISPR system. The art is replete with suitable vectors that are useful in the present invention. The vectors to be used are suitable for replication and, optionally, integration in eukaryotic cells. Typical vectors contain transcription and translation terminators, initiation sequences, and promoters useful for regulation of the expression of the desired nucleic acid sequence. The vectors of the present invention may also be used for nucleic acid standard gene delivery protocols. Methods for gene delivery are known in the art (U.S. Pat. Nos. 5,399,346, 5,580,859 & 5,589,466, incorporated by reference herein in their entireties).

Further, the vector may be provided to a cell in the form of a viral vector. Viral vector technology is well known in the art and is described, for example, in Sambrook et al. (4^{th} Edition, Molecular Cloning: A Laboratory Manual, Cold Spring Harbor Laboratory, New York, 2012), and in other virology and molecular biology manuals. Viruses, which are useful as vectors include, but are not limited to, retroviruses, adenoviruses, adeno-associated viruses, herpes viruses, Sindbis virus, gammaretrovirus and lentiviruses. In general, a suitable vector contains an origin of replication functional in at least one organism, a promoter sequence, convenient restriction endonuclease sites, and one or more selectable markers (e.g., WO 01/96584; WO 01/29058; and U.S. Pat. No. 6,326,193).

### Introduction of Nucleic Acids

Methods of introducing nucleic acids into a cell include physical, biological and chemical methods. Physical methods for introducing a polynucleotide, such as RNA, into a host cell include calcium phosphate precipitation, lipofection, particle bombardment, microinjection, electroporation, and the like. RNA can be introduced into target cells using commercially available methods which include electroporation (Amaxa Nucleofector-II (Amaxa Biosystems, Cologne, Germany)), (ECM 830 (BTX) (Harvard Instruments, Boston, Mass.) or the Gene Pulser II (BioRad, Denver, Colo.), Multiporator (Eppendort, Hamburg Germany). RNA can also be introduced into cells using cationic liposome mediated transfection using lipofection, using polymer encapsulation, using peptide mediated transfection, or using biolistic particle delivery systems such as “gene guns” (see, for example, Nishikawa, et al. Hum Gene Ther., 12(8):861-70 (2001).

Biological methods for introducing a polynucleotide of interest into a host cell include the use of DNA and RNA vectors. Viral vectors, and especially retroviral vectors, have become the most widely used method for inserting genes into mammalian, e.g., human cells. Other viral vectors can be derived from lentivirus, poxviruses, herpes simplex virus I, adenoviruses and adeno-associated viruses, and the like. See, for example, U.S. Pat. Nos. 5,350,674 and 5,585,362.

Chemical means for introducing a polynucleotide into a host cell include colloidal dispersion systems, such as macromolecule complexes, nanocapsules, microspheres, beads, and lipid-based systems including oil-in-water emulsions, micelles, mixed micelles, and liposomes. An exemplary colloidal system for use as a delivery vehicle in vitro and in vivo is a liposome (e.g., an artificial membrane vesicle).

Regardless of the method used to introduce exogenous nucleic acids into a host cell or otherwise expose a cell to the inhibitor of the present invention, in order to confirm the presence of the nucleic acids in the host cell, a variety of assays may be performed. Such assays include, for example, “molecular biological” assays well known to those of skill in the art, such as Southern and Northern blotting, RT-PCR and PCR; “biochemical” assays, such as detecting the presence or absence of a particular peptide, e.g., by immunological means (ELISAs and Western blots) or by assays described herein to identify agents falling within the scope of the invention.

It should be understood that the method and compositions that would be useful in the present invention are not limited to the particular formulations set forth in the examples. The following examples are put forth so as to provide those of ordinary skill in the art with a complete disclosure and description, and are not intended to limit the scope of what the inventors regard as their invention.

The practice of the present invention employs, unless otherwise indicated, conventional techniques of molecular biology (including recombinant techniques), microbiology, cell biology, biochemistry and immunology, which are well within the purview of the skilled artisan. Such techniques are explained fully in the literature, such as, Molecular Cloning: A Laboratory Manual”, fourth edition (Sambrook et al. (2012) Molecular Cloning, Cold Spring Harbor Laboratory); “Oligonucleotide Synthesis” (Gait, M. J. (1984). Oligonucleotide synthesis. IRL press); “Culture of Animal Cells” (Freshney, R. (2010). Culture of animal cells. Cell Proliferation, 15(2.3), 1); “Methods in Enzymology” “Weir's Handbook of Experimental Immunology” (Wiley-Blackwell; 5 edition (Jan. 15, 1996); “Gene Transfer Vectors for Mammalian Cells” (Miller and Carlos, (1987) Cold Spring Harbor Laboratory, New York); “Short Protocols in Molecular Biology” (Ausubel et al., Current Protocols; 5 edition (Nov. 5, 2002)); “Polymerase Chain Reaction: Principles, Applications and Troubleshooting”, (Babar, M., VDM Verlag Dr. Müller (Aug. 17, 2011)); “Current Protocols in Immunology” (Coligan, John Wiley & Sons, Inc. Nov. 1, 2002).

Those skilled in the art will recognize, or be able to ascertain using no more than routine experimentation, numerous equivalents to the specific procedures, embodiments, claims, and examples described herein. Such equivalents were considered to be within the scope of this invention and covered by the claims appended hereto. For example, it should be understood, that modifications in reaction conditions, including but not limited to reaction times, reaction size/volume, and experimental reagents, such as solvents, catalysts, pressures, atmospheric conditions, e.g., nitrogen atmosphere, and reducing/oxidizing agents, with art-recognized alternatives and using no more than routine experimentation, are within the scope of the present application.

It is to be understood that wherever values and ranges are provided herein, all values and ranges encompassed by these values and ranges, are meant to be encompassed within the scope of the present invention. Moreover, all values that fall within these ranges, as well as the upper or lower limits of a range of values, are also contemplated by the present application.

The following examples further illustrate aspects of the present invention. However, they are in no way a limitation of the teachings or disclosure of the present invention as set forth herein.

### EXPERIMENTAL EXAMPLES

The invention is now described with reference to the following Examples. These Examples are provided for the purpose of illustration only, and the invention is not limited to these Examples, but rather encompasses all variations that are evident as a result of the teachings provided herein.

The materials and methods employed in these experiments are now described.

Mice: Mice, both sexes, between the ages of 6-12 weeks of age were used for the study. OT-I TCR transgenic mice (OT-I mice) were described by Hogquist et al. (1994) Cell 76, 17-27. Constitutive Cas9-2A-EGFP mice (Cas9 mice) were described by Chu et al. (2016) BMC Biotechnol 16, 4.; Platt et al. (2014) Cell 159, 440-455. OT-I; Cas9 mice were generated by breeding OT-I and Cas9 mice, and genotyped according to Jackson Lab protocol. Naive CD8⁺ T cells were isolated from OT-I mice, Cas9 mice, and OT-I; Cas9 mice. All animals were housed in standard individually ventilated, pathogen-free conditions, with 12 h:12 h or 13 h:11 h light cycle, room temperature (21-23° C.) and 40-60% relative humidity. When a cohort of animals were receiving multiple treatments, animals were randomized by 1) randomly assigning animals to different groups using littermates, 2) random mixing of females prior to treatment, maximizing the evenness or representation of mice from different cages in each group, and/or 3) random assignment of mice to each group, in order to minimize the effect of gender, litter, small difference in age, cage, housing position, where applicable.

Generation of a T cell CRISPR knockout vector (sgRNA-Thy1.1 Expression Vector): A lentiviral T cell CRISPR knockout vector, lenti-pLKO-U6-sgRNA(BsmBI)-EFS-Thy1.1CO-spA, was generated by codon-optimizing and subcloning Thy1.1 and sgRNA expression cassette into a lentiviral vector via Gibson Assembly.

Genome-scale mouse T cell CRISPR knockout library cloning: The original mouse CRISPR knockout library, in two sub-libraries (mGeCKOa and mGeCKOb) was from Sanjana et al. (2014) Nat Methods 11, 783-784. mGeCKOa and mGeCKOb were sub-cloned in equal molar, by Gibson assembly and electroporation, into the T cell CRISPR vector to generate the Genome-scale mouse T cell CRISPR knockout library (MKO), with a total of 129,209 sgRNAs including 1,000 non-targeting controls (NTCs). An estimated library coverage of >50× (˜7×10⁶ total colonies) was achieved in electroporation. The library was subsequently sequence-verified by Illumina sequencing. At least 94.1% (121,608/129,209) of unique sgRNAs the whole library cloned, targeting 98.3% (22,375/22,768) of all protein coding genes and microRNAs in the mouse genome, with a tight log-normal distribution representing the vast majority of all designed sgRNAs (90% within 2 orders of magnitude, 99% within 3 orders of magnitude).

Viral library production: The MKO library plasmid was transfected into low-passage HEK293FT cells at 80% confluency in 15 cm tissue culture plates. Viral supernatant was collected at 48 h and 72 h post-transfection, filtered via a 0.45 m filtration unit (Fisher/VWR), and concentrated using AmiconUltra 100 kD ultracentrifugation units (Millipore), aliquoted and stored in −80° C. until use. Virus for empty vector was produced in a similar manner.

T cell isolation and culture: Spleens and mesenteric lymph nodes (mLNs) were isolated from various indicated mouse strains, and placed in ice-cold 2% FBS [FBS (Sigma)+RPMI-1640 (Lonza)]. Organs were prepared by mashing through a 100 μm filter. Lymphocytes were suspended in 2% FBS. RBCs were lysed with 1 ml of ACK Lysis Buffer (Lonza) per spleen, incubated for 2 mins at room temperature, and washed with 2% FBS. Lymphocytes were filtered through a 40 μm filter and resuspended with MACS Buffer (PBS+2% FBS+2 μM EDTA). Naive CD8⁺ T cells were isolated using the protocol and kit established by Miltenyi. Naive CD8⁺ T cells were resuspended with cRPMI (RPMI-1640+10% FBS+2 mM L-Glutamine+100 U Pen/Strep (Fisher)+49 nM f-mercaptoethanol (Sigma)) to a final concentration of 1×10⁶ cells/ml. Medium for in vivo experiments was supplemented with 2 ng/ml IL-2+2.5 ng/ml IL-7+50 ng/ml IL-15+1 μg/ml anti-CD28. Medium for in vitro experiments was supplemented with 2 ng/ml IL-2+2 ng/ml IL-12p70+1 μg/ml anti-CD28. Cells were cultured on plates pretreated with 5 μg/ml anti-CD3 and incubated at 37° C. Cytokines and antibodies mentioned above were purchased from BD, Biolegend and eBiosciences.

T cell transduction, virus titration: T cells were infected in culture immediately after isolation by directly adding concentrated virus into the media. Three days after infection, T cells were stained for Thy1.1 expression and analyzed on FACS. Viral titer was determined for each batch by the number of Thy1.1⁺ T cells normalized to total T cells divided by the volume of virus used. At least 3 doses of viruses with experimental duplicates were used for determining viral titer.

Antibody and Flow Cytometry: Infectivity of CD8⁺ T cells was assessed via surface staining with anti-CD3 APC, anti-CD8a FITC, and anti-Thy1.1 PE (BioLegend). Cells were stained on ice for 30 mins. Samples were collected on a BD FACSAria cell sorter with 3 lasers, and analyzed using FlowJo software 9.9.4 (Treestar, Ashland, Oreg.) on a MAC® workstation.

Library-scale viral transduction of T cells: T cells were isolated and cultured as described herein. With the viral titer information, for each infection replicate, a total of >1×10⁸ Cas9 or naive OT-I; Cas9 CD8⁺ T cells were transduced at a MOI of 1 with concentrated lentivirus containing the MKO library described above, to achieve an initial library coverage of >700×. Transduction with the virus containing the empty vector was performed in parallel with a total of >1×10⁷ naive CD8⁺ T cells.

Adoptive transfer of viral library infected T cells and tissue processing: At day 0 of the culture, naive CD8⁺ T cells were infected with the lentiviral MKO library, and incubated at 37° C. for 3 days. On day 3 of culture, T cells were collected, washed with ice-cold PBS, and resuspended to a final concentration of 5×10⁷ cells/ml. 1×10⁷ cells were injected intravenously into each mouse. C57BL/6 (B6), Cas9, or Rag1^{-/-} mice were used as recipient mice in respective experiments. On 7-day post-transfer, mice were euthanized, and relevant organs were isolated. Skin draining lymph nodes were comprised of inguinal, popliteal, axillary, and brachial lymph nodes. Cervical lymph nodes were comprised of the 6 superficial cervical lymph nodes. Abdominal lymph nodes were comprised of the mesenteric and pancreatic lymph nodes. Other relevant organs isolated were the spleen, liver, pancreas, lung, muscle and brain.

Generation of a neoantigen expression vector (mCherry-cOVA Expression Vector): A lentiviral mCherry-cOVA (mCh-cOVA) vector, lenti-pLKO-U6-sg(BsmBI)-EFS-mCherry-2A-cOVA, was generated by subcloning cOVA into a mCherry lentiviral vector via Gibson Assembly.

Generation of stably transfected mCherry-cOVA expressing cell line: E0771 murine breast cancer cells were transduced with mCh-cOVA-expressing lentivirus. After 3 days post-transduction, transduced E0771 cells were cultured individually in 96-well plate by resuspending cells to 10 cells/ml and culturing 100 μl of cell suspension in each well. 2 weeks later, clonal mCh⁺ E0771 clones were identified by fluorescence microscopy. mCh⁺ E0771 clones were stained with established anti-mouse [SIINFEKL: H-2K^{b}] antibody to determine cOVA expression. Different mCh⁺cOVA⁺ clones were selected based on cOVA expression. Clone 3 was chosen for in vivo experiments because of its low, uniform expression of cOVA to select for genes with stronger phenotypes.

Transplantation of cancer cells into Rag1^{-/-} mice and tissue processing: 5×10⁶ mCh⁺cOVA⁺ E0771 cells were either injected either subcutaneously or into the intra-mammary fat pad of Rag1^{-/-} mice. 10 days post-transplantation, viral library infected T cells were intravenously injected in tumor-bearing Rag1^{-/-} mice. After 7 days, draining lymph nodes, non-draining lymph nodes, spleens, lungs, and tumors were isolated. Samples were prepared for DNA extraction or FACS analysis. Tumors were broken down into smaller fragments, about the size of lentils. Tumors were then dissociated with 1 μg/ml Collagenase IV for 30 minutes using GentleMacs Octo dissociator from Miltenyi, and cell suspensions were passed through 100 μm filter twice before staining.

Degranulation assay and genome-scale CRISPR screening: Experiments were first optimized by pulsing E0771 cells with varying concentrations of SIINFEKL peptide for 4 hours at 37° C., and subsequently stained with the anti-mouse [SIINFEKL: H-2K^{b}] antibody and analyzed on flow cytometry. The dose of 1 ng/ml was chosen as it represents the maximum concentration tested without being detected by anti-(SIINFEKL: H-2K^{b}). Naive OT-I; Cas9 CD8⁺ T cells were isolated and transduced with MKO lentiviral library. Infected OT-I; Cas9 CD8⁺ T cells were incubated on plates pretreated with 5 μg/ml anti-CD38 in cRPMI supplemented with 2 ng/ml IL-2+2 ng/ml IL-12p70+1 μg/ml anti-CD28 for 6 days. 12 hours before the assay, infected OT-I; Cas9 CD8⁺ T cells were incubated on untreated plates in the presence of 2 ng/ml IL-2+2 ng/ml IL-12 p70 to rest the cells. On day 6, 12 hours before the assay, 1×10⁷ E0771 cells were also plated on 10 cm plate in D10 media (DMEM+10% FBS+100 U Pen/Strep). The following day, E0771 cells were incubated with warm D10 media supplemented with either 0 or 1 ng/ml SIINFEKL peptide for 4 hours. Meanwhile, infected OT-I; Cas9 CD8⁺ T cells were resuspended to a final concentration 1×10⁶ cells/ml with cRPMI+2 nM monensin+anti-CD107a PE antibody, and added to E0771 cells at a T cell: seeding cancer cell ratio=1:1. Cells were coincubated at 37° C. for 2 hours. Cells were then stained with anti-CD8 APC for 30 minutes on ice, and cells were sorted via BD FACSAria. A total of 1×10⁷ T cells were analyzed, and the top 5% CD107a⁺ cells were sorted, and subjected to genomic DNA extraction, CRISPR library readout, and screen data analysis. A total of three biological replicates were performed.

Genomic DNA extraction from cells and mouse tissues: For gDNA extraction, three methods were used. Method 1: for samples with a total number of less than or equal to 1×10⁵ cells, 100 μl of QuickExtract solution (Epicentre) was directly added to cells and incubated at 65° C. for 30 to 60 minutes until the cell pellets were completely dissolved. Method 2: for cellular samples with a total number of 1×10⁵ to 2×10⁶ cells, or tissue samples from mouse lymph nodes, samples were subjected to QIAamp Fast DNA Tissue Kit (Qiagen) following the manufacturer's protocol. Method 3: for cellular samples with a total number of greater than 2×10⁶ cells, or tissue samples from mouse organs such as spleen, lung, liver, brain, pancreas, colon, or tumor samples, a custom Puregene protocol was used. Briefly, 50-200 mg of frozen ground tissue were resuspended in 6 ml of Lysis Buffer (50 mM Tris, 50 mM EDTA, 1% SDS, pH 8) in a 15 ml conical tube, and 30 μl of 20 mg/ml Proteinase K (Qiagen) were added to the tissue/cell sample and incubated at 55° C. overnight. The next day, 30 μl of 10 mg/ml RNAse A (Qiagen) was added to the lysed sample, which was then inverted 25 times and incubated at 37° C. for 30 minutes. Samples were cooled on ice before addition of 2 ml of pre-chilled 7.5M ammonium acetate (Sigma) to precipitate proteins. The samples were vortexed at high speed for 20 seconds and then centrifuged at ≥4,000×g for 10 minutes. Then, a tight pellet was visible in each tube and the supernatant was carefully decanted into a new 15 ml conical tube. Then 6 ml 100% isopropanol was added to the tube, inverted 50 times and centrifuged at ≥4,000×g for 10 minutes. Genomic DNA was visible as a small white pellet in each tube. The supernatant was discarded, 6 ml of freshly prepared 70% ethanol was added, the tube was inverted 10 times, and then centrifuged at ≥4,000×g for 1 minute. The supernatant was discarded by pouring; the tube was briefly spun, and remaining ethanol was removed using a P200 pipette. After air-drying for 10-30 minutes, the DNA changed appearance from a milky white pellet to slightly translucent. Then, 500 μl of ddH₂O was added, the tube was incubated at 65° C. for 1 hour and at room temperature overnight to fully resuspend the DNA. The next day, the gDNA samples were vortexed briefly. The gDNA concentration was measured using a Nanodrop (Thermo Scientific).

SgRNA library readout by deep sequencing: The sgRNA library readout was performed using a two-steps PCR strategy, where the first PCR includes enough genomic DNA to preserve full library complexity and the second PCR adds appropriate sequencing adapters to the products from the first PCR.

For PCR #1, a region containing sgRNA cassette was amplified using primers specific to the T cell CRISPR knockout vector:

Forward
(SEQ ID NO: 3032)
CCCGAGGGGACCCAGAGAG
Reverse
(SEQ ID NO: 3033)
CAATTCCCACTCCTTTCAAGAC

PCR was performed using Phusion Flash High Fidelity Master Mix (PF) or DreamTaq Green PCR Master Mix (DT) (ThermoFisher). For reactions using PF, in PCR #1, the thermocycling parameters were: 98° C. for 2 min, 18-24 cycles of (98° C. for 1 s, 62° C. for 5 s, 72° C. for 30 s), and 72° C. for 2 minute. For reactions using DT, the thermocycling parameters were adjusted according to manufacturer's protocol. In each PCR #1 reaction, we used 3 μg of total gDNA. For each sample, the appropriate number of PCR #1 reactions was used to capture the full representation of the screen. For example, at ˜200× coverage of our 129,209 MKO sgRNA library, gDNA from 2.5×10⁷ cells was used. Assuming 6.6 μg of gDNA per cell, ˜160 μg of gDNA was used per sample, in approximately 50 PCR #1 reactions (with ˜3 μg of gDNA per reaction).

PCR #1 products for each biological sample were pooled and used for amplification with barcoded second PCR primers. For each sample, at least 4 PCR #2 reactions were performed using 2 μl of the pooled PCR #1 product per PCR #2 reaction. Second PCR products were pooled and then normalized for each biological sample before combining uniquely barcoded separate biological samples. The pooled product was then gel purified from a 2% E-gel EX (Life Technologies) using the QiaQuick kit (Qiagen). The purified pooled library was then quantified with a gel-based method using the Low-Range Quantitative Ladder Life Technologies, dsDNA High-Sensitivity Qubit (Life Technologies), BioAnalyzer (Agilent) and/or qPCR. Diluted libraries with 5-20% PhiX were sequenced with MiSeq, HiSeq 2500 or HiSeq 4000 systems (Illumina).

Demultiplexing and readpreprocessing: Raw single-end fastq read files were filtered and demultiplexed using Cutadapt (Martin, (2011) EMBnetjournal 17, 10-12). To remove extra sequences downstream (i.e. 3′ end) of the sgRNA spacer sequences, the following settings were used: cutadapt --discard-untrimmed -a GTTTTAGAGCTAGAAATGGC (SEQ ID NO: 3034). As the forward PCR primers used to readout sgRNA representation were designed to have a variety of barcodes to facilitate multiplexed sequencing, these filtered reads were then demultiplexed with the following settings: cutadapt -g file:fbc.fasta --no-trim, where fbc.fasta contained the 12 possible barcode sequences within the forward primers. Finally, to remove extra sequences upstream (i.e. 5′ end) of the sgRNA spacers, we used the following settings: cutadapt --discard-untrimmed -g GTGGAAAGGACGAAACACCG (SEQ ID NO: 3035). Through this procedure, the raw fastq read files could be pared down to the 20 bp sgRNA spacer sequences.

Mapping of sgRNA spacers and quantitation of sgRNAs: Having extracted the 20 bp sgRNA spacer sequences from each demultiplexed sample, the sgRNA spacers were then mapped to the MKO library. To do so, a bowtie index was generated of either sgRNA library using the bowtie-build command in Bowtie 1.1.2 (Langmead et al. (2009) Genome Biol 10, R25). Using these bowtie indexes, the filtered fastq read files were mapped using the following settings: bowtie -v 1 --suppress 4,5,6,7 --chunkmbs 2000 -best. Using the resultant mapping output, the number of reads that had mapped to each sgRNA within the library were quantitated. To generate sgRNA representation barplots, a detection threshold of 1 read was set, and the number of unique sgRNAs present in each sample was counted.

Normalization and summary-level analysis of sgRNA abundances: The number of reads in each sample was normalized by converting raw sgRNA counts to reads per million (rpm). The rpm values were then subject to log₂ transformation for certain analyses. To generate correlation heatmaps, the NMF R package (Gaujoux and Seoighe, (2010) BMC Bioinformatics 11, 367) was used and calculated the Pearson correlations between individual samples using log 2 rpm counts. To calculate the cumulative distribution function for each sample group, the normalized sgRNA counts were first averaged across all samples within a given group. The ecdfplot function in the latticeExtra R package was used to generate empirical cumulative distribution plots.

Enrichment analysis of sgRNAs: Three criteria were used to identify the top candidate genes: 1) if an sgRNA comprised ≥2% of the total reads in at least one organ sample; 2) if an sgRNA was deemed statistically significantly enriched in ≥20% of all organ samples using a false-discovery rate (FDR) threshold of 0.5% based on the abundances of all non-targeting controls; or 3) if ≥2 independent sgRNAs targeting the same gene were each found to be statistically significant at FDR<0.5% in at least one sample each. For the first and second criteria, individual sgRNA hits were collapsed to genes to facilitate comparisons with the hits from the third criteria.

Heatmap sgRNA library representation: Heatmaps of the top enriched sgRNAs were generated using the a heatmap function with default setting (NMF R package). Only sgRNAs with a log₂ rpm≥1 were included for visualization in the heatmaps.

Overlap and significance analysis of enriched sgRNAs: To generate Venn diagrams of overlapping enriched sgRNAs or genes, all sgRNAs were considered that were found to be significant across different statistical calling algorithms, different T cells, or different experiments.

Gene ontology and pathway enrichment analysis: Various gene sets were used for gene ontology and pathway enrichment analysis using DAVID functional annotation analysis (Huang et al., (2009) Nucleic Acids Res 37, 1-13). For sgRNA set, sgRNAs were converted to their target genes and then the resultant genes were used for analysis.

Testing anti-tumor function of T cells with sgRNAs targeting individual genes by adoptive transfer: SgRNAs targeting individual genes were cloned into the T cell CRISPR vector. Two independent sgRNAs targeting each gene (e.g. Dhx37) were used (SEQ ID NOs: 1-10). Virus prep and T cell infection were performed as described herein. 5×10⁶ mCh⁺cOVA⁺ E0771 cells were injected either subcutaneously or into the intra-mammary fat pad of Rag1^{-/-} mice. 7 days post-transplantation, freshly isolated naive OT-I; Cas9 CD8⁺ T cells were plated on plates pretreated with 5 μg/ml anti-CD38 in cRPMI supplemented with 2 ng/ml IL-2+2.5 ng/mL IL-7+50 ng/mL IL-15+1 μg/ml anti-CD28, infected with these sgRNA-containing lentiviruses (at MOI of ˜1) as described herein, and cultured for 3 days. 10 days post-transplantation, 5×10⁶ virally infected T cells were intravenously injected in tumor-bearing Rag1^{-/-} mice (T cell: initial cancer cell ratio=1:1). PBS and empty vector infected T cells were used as adoptive transfer controls. Tumor sizes were measured by caliper once to twice per week. 6 weeks after adoptive transfer, tumors were dissected, and samples were subjected to molecular, cellular, histology analysis, or single-cell RNA-seq. For statistical comparison of tumor growth curves, multiple t-tests were performed (Benjamini, Krieger and Yekutieli FDR method) on each timepoint.

Tumor Infiltration Lymphocyte (TIL) Isolation for single cell RNA-seq: Tumor bearing mice were euthanized at designated time points, and their tumors were collected and kept in ice cold 2% FBS. Tumors were minced into 1-3 mm size pieces using scalpel and then digested in 1 μg/ml Collagenase IV for 30-60 min using Miltenyi GentleMACS Octo Dissociator. Tumor suspensions were filtered twice through 100 μm cell strainer, and again through 40 μm cell strainer to remove large bulk. Subsequently, tumor suspensions were carefully layered onto Ficoll-Paque media (GE Healthcare) and centrifuge at 400 g for 30 min to enrich lymphocytes at the bilayer interface. Cells at the interface were carefully collected, and washed twice with 2% FBS, counted, and stained with indicated antibodies for 30 minutes on ice. CD3⁺CD8⁺ TILs were then sorted on BD FACSAria. A total of 3×10³ to 2×10⁴ TILs were collected per tumor.

TIL single cell RRNA-seq (scRNAseq): TILs sorted from freshly isolated tumors were subjected to single-cell RNAseq library prep. A protocol by 10× Genomics was followed. In brief, Single Cell Master Mix was prepared fresh containing RT reagent mix, RT primer, additive A, and RT enzyme mix. A Single Cell 3′ Chip was placed in a 10×™ Chip Holder. 50% glycerol solution to each unused well accordingly, TIL solution at ˜100 cell/ul was added together with the master mix. The Single Cell 3′ Gel Bead Strip was placed into a 10×™ Vortex Adapter and vortex for 30 sec. Then, Single Cell 3′ Gel Bead suspension and Partitioning Oil were dispensed into the bottom of the wells in the specified rows. The fully loaded chip was then inserted into Chromium™ Controller to generate emulsion. The emulsion was then transferred to a 96-well PCR plate for GEM-RT reaction, RT clean up, cDNA amplification, cDNA clean up, quantification and QC, and subjected to Illumina library construction. In library construction, clean input cDNA was then subjected to fragmentation, end repair & A-tailing. After that, double sided size selection was performed using SPRI Select, followed by adaptor ligation, clean up, and sample indexing PCR, pooling and PCR cleanup, resulting a single-cell RNA-seq library. Enzymatic Fragmentation and Size Selection were used to optimize the cDNA amplicon size prior to library construction per manufacturer's protocols. R1 (read 1 primer sequence) are added to the molecules during GEM incubation. P5, P7, a sample index and R2 (read 2 primer sequence) are added during library construction via end repair, A-tailing, adaptor ligation and PCR. The Single Cell 3′ Protocol produces Illumina-ready sequencing libraries contain the P5 and P7 primers used in Illumina bridge amplification. This final library was then QC'ed and quantified using BioAnalyzer, and loaded on a Hiseq 2500 RapidRun for standard Illumina paired-end sequencing, where Barcode and 10 bp randomer (UMI) is encoded in Read 1, while Read 2 is used to sequence the cDNA fragment. Sample index sequences are incorporated as the i7 index read.

TIL scRNA-seq dataprocessing: TIL scRNA-seq_fastq data was pre-processed using established and custom pipelines. Briefly, raw Illumina data files were subjected to Cell Ranger, which used cellranger mkfastq to wrap Illumina's bcl2fastq to correctly demultiplex Chromium-prepared sequencing samples and to convert barcode and read data to FASTQ files. Then, cellranger count was used to take FASTQ files and performs alignment to mouse genome (mm10), filtering, and UMI counting. Raw sequencing output was first preprocessed by Cell Ranger 1.3 (10× Genomics) (Zheng et al., (2017) Nat Commun 8, 14049) using cellranger mkfastq, count, and aggr (no normalization mode). Cells passed the initial quality control metrics imposed by the Cell Ranger pipeline were further filtered using a variety of criteria (Lun et al., (2016) F1000Res 5, 2122): 1) All cells with a total library count (i.e. # of UMIs) that was ≥4 standard deviations below the mean were excluded; 2) All cells with library diversity (i.e. # of detected genes/features) that was ≥4 standard deviations below the mean were excluded; and 3) All cells in which mitochondrial genes disproportionately comprised the total % of the library (≥4 standard deviations above the mean) were excluded. After applying these 3 filters, a final set of cells was retained for further analysis. The 27,998 genes/features were additionally filtered using a flat cutoff metric: genes with an average count of <0.05 across the 12 dataset were excluded. Finally, the data was normalized by library size using the scran R package (Lun et al., (2016) F1000Res 5, 2122).

scRNA-seq t-SNE dimension reduction and visualization: Using the final normalized and processed dataset, t-SNE dimension reduction was performed using the Rtsne R package with default settings (Maaten, (2014) J Mach Learn Res 15, 3221-3245). Individual data points were colored based on the treatment condition for each cell.

scRNA-seq differential expression analysis: Using the final normalized and processed dataset, differential expression analysis was performed using the edgeR R package (Robinson et al., (2010) Bioinformatics 26, 139-140). In brief, edgeR first estimates the negative binomial dispersion parameter to model the variance between cells from the same treatment group. A generalized linear model is then fitted to determine differentially expressed genes between treatment conditions. Multiple hypothesis correction was performed by the Benjamini-Hochberg method. Significantly differentially expressed genes were defined as having a Benjamini-Hochberg adjusted p<0.05, with upregulated genes having a positive log fold change and downregulated genes having a negative log fold change. Volcano plots were generated using edgeR output statistics. Gene ontology enrichment analyses on differentially expressed genes were performed using the PANTHER classification system (Mi et al., (2013) Nat Protoc 8, 1551-1566). The statistical overrepresentation test was used to identify enriched GO (biological process) categories among the differentially expressed genes. Bonferroni multiple hypothesis correction was performed.

scRNA-seq heatmap of differentially expressed genes: To generate an overall view of the top differentially expressed genes, the genes with an absolute log fold change ≥1 were selected. each row of the dataset was then scaled (i.e. by gene) to obtain z-scores. To improve visibility in the heatmap, the dynamic range of the z-scores was compressed to a maximum of 6 (denoted as 6+). Heatmaps were generated using the NMF R package (Gaujoux and Seoighe, (2010) BMC Bioinformatics 11, 367).

Blinding statement: Investigators were blinded for sequencing data analysis, but not blinded for tumor engraftment, adoptive transfer, organ and tumor dissection, and flow cytometry.

The results of the experiments are now described.

### Example 1: Genome-Scale T Cell Knockout Library and Genetic Screen for Trafficking and Survival in CD8+ T Cells with Diverse TCR

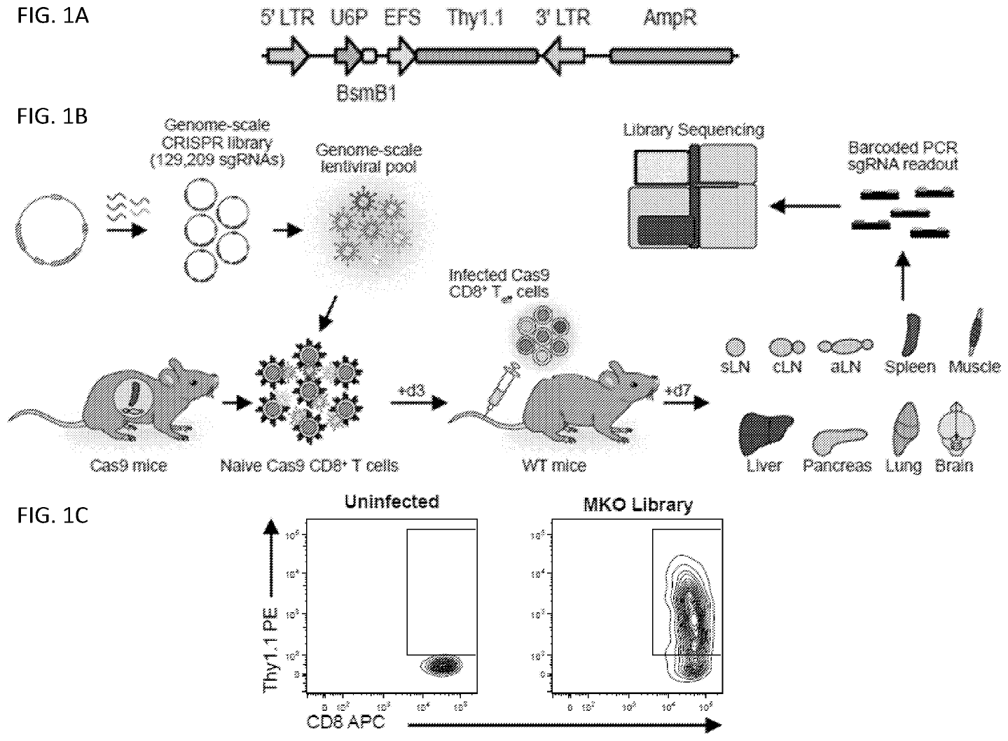

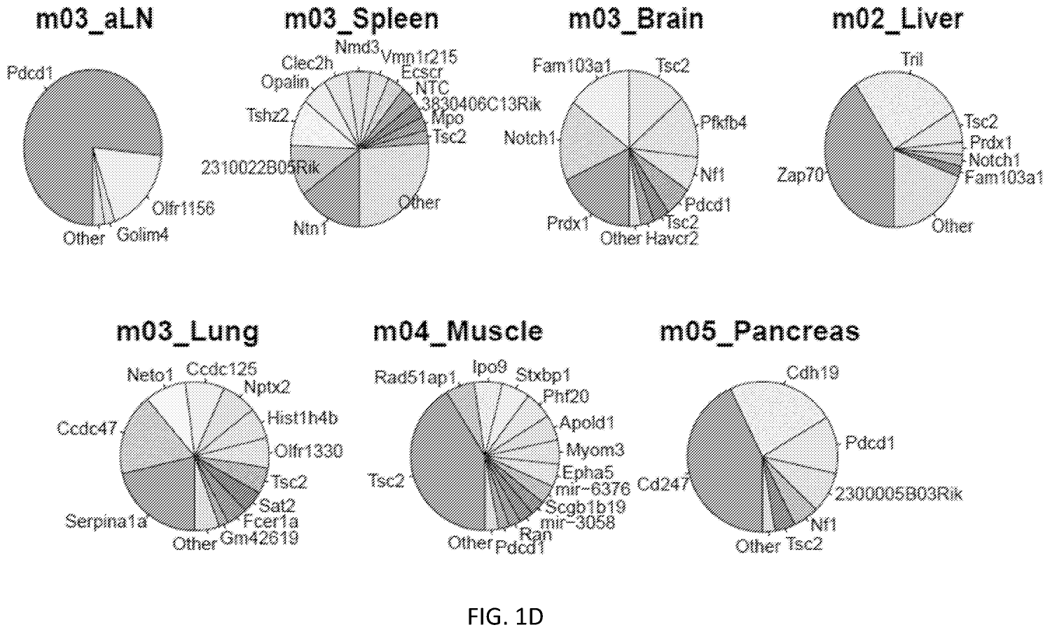

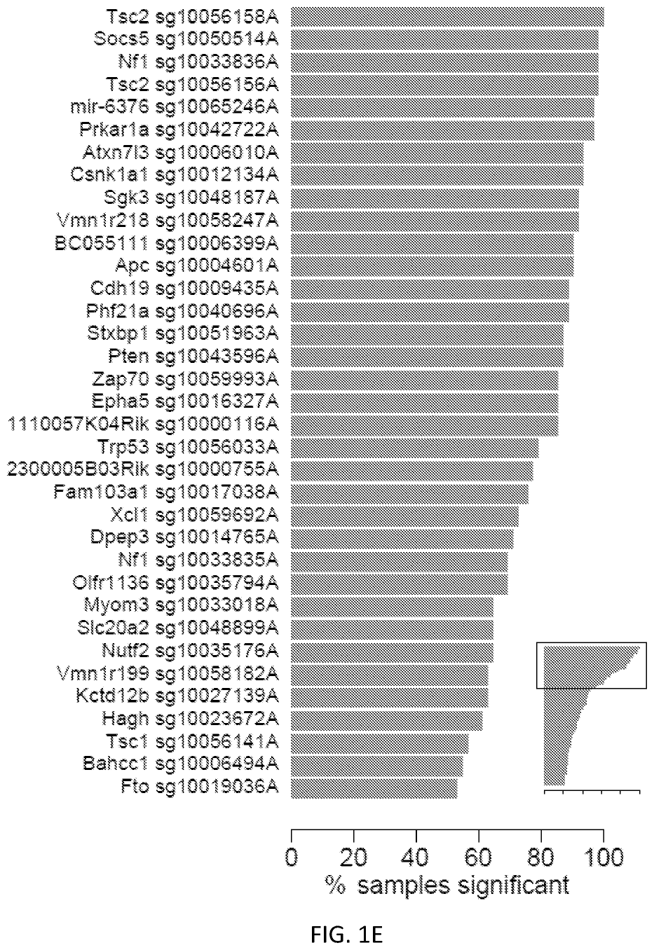

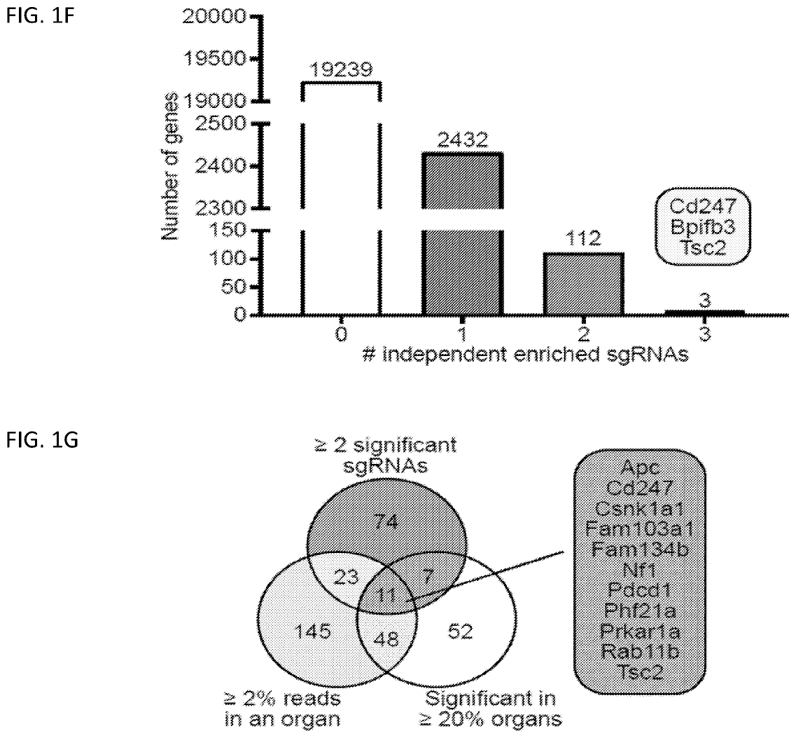

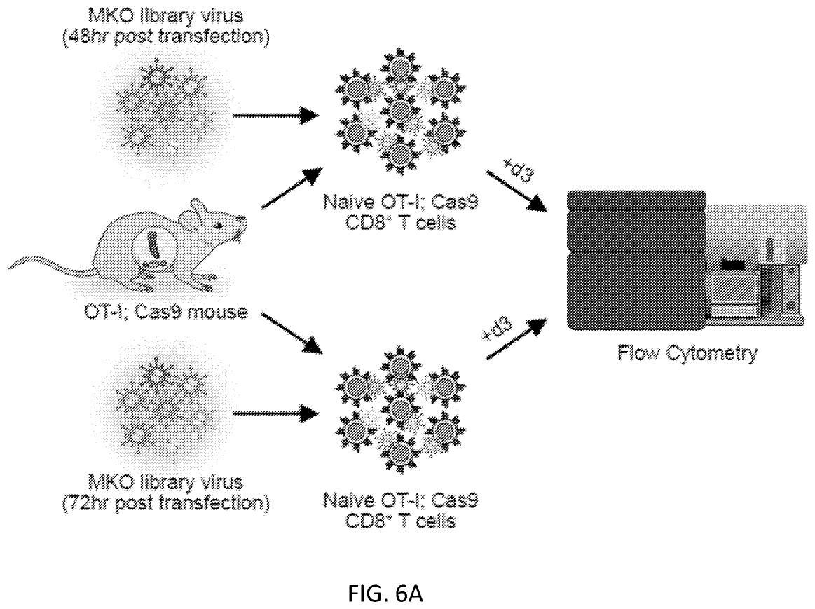

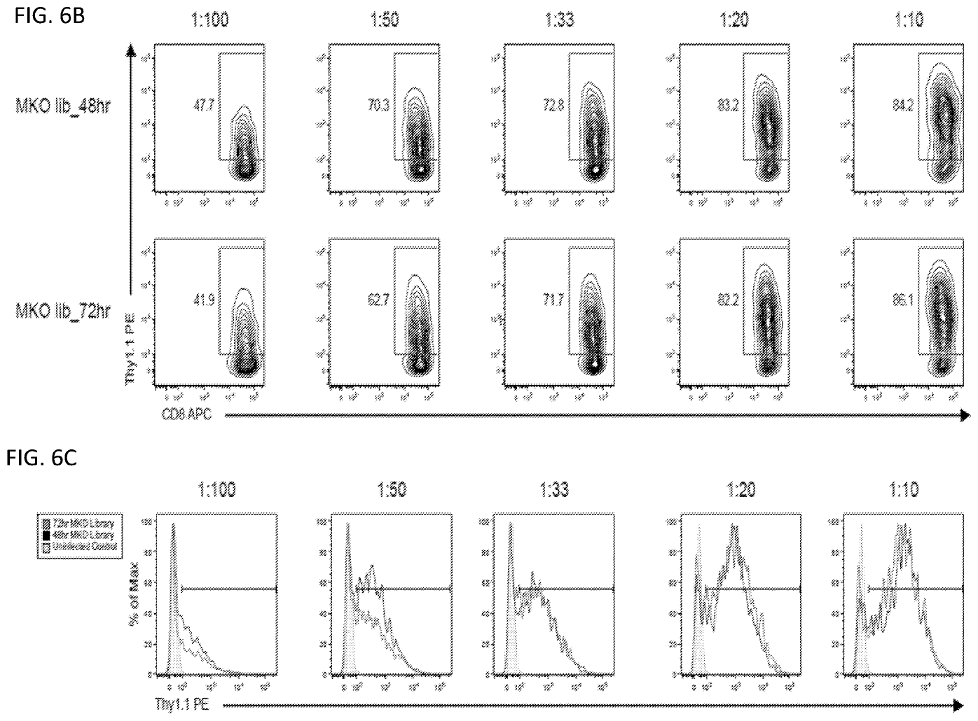

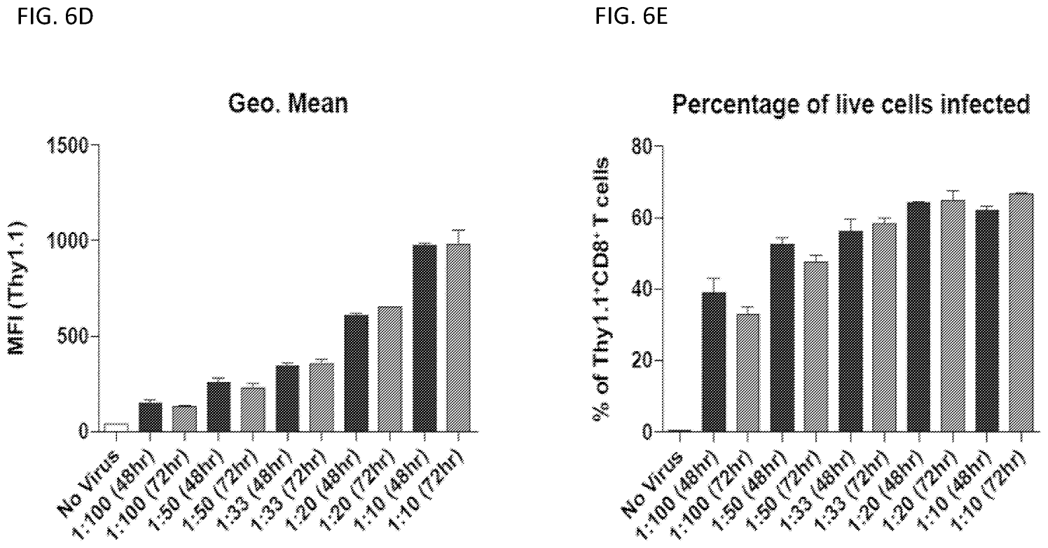

To enable CRISPR screen in CD8⁺ T cells, a T cell knockout vector was designed and generated. This vector contained an sgRNA expression cassette enabling genome editing in conjunction with Cas9, and a cassette that expresses a congenic variant of Thy1 protein (Thy1.1) for specific identification and single-cell isolation of transduced CD8⁺ T cells (FIG. 1A). In order to conduct large-scale genetic manipulation and thus high-throughput screening, a genome-scale sgRNA library was cloned into the vector. The sgRNA library contained a total of 129,209 sgRNAs including 128,209 sgRNAs each targeting a gene in the mouse genome, and 1,000 non-targeting controls (NTCs), at an estimated library coverage of >50× (˜7×10⁶ total colonies). Successful cloning of the library was verified (tight log-normal distribution of designed sgRNAs, covering 98.3% targeted genes) by Illumina sequencing. High-titer lentivirus was generated from this sgRNA library (termed MKO thereafter), and it was tested whether they could efficiently transduce cytotoxic T cells. Naive CD8⁺ T cells were isolated from mice that constitutively express Cas9, enabling genetic perturbations upon delivery of sgRNA. T cells were transduced with various concentrations of MKO virus, and analyzed the expression of the Thy1.1 surface marker via flow cytometry three days post-infection (FIG. 1B, FIG. 6A). Efficient transduction of CD8⁺ T cells was detected with various concentrations of MKO virus (FIG. 1C, FIGS. 6B-6E).

To map the genetic factors modulating the trafficking and survival of diverse T cell populations in vivo, the MKO library was used to interrogate the survival of adoptively transferred mutant T cells after trafficking to relevant organs (FIG. 1). First, freshly isolated naive Cas9 CD8⁺ T cells were mutagenized by transducing with the MKO lentiviral sgRNA library to achieve a coverage of >700× for the initial population, with 3 infection replicates. Three days after transduction, the MKO-infected mutant pool of CD8⁺ T cells (MKO T cell library) were adoptively transferred into wildtype C57BL/6 (B6) recipient mice (n=7) (FIG. 1C). It is expected that after adoptive transfer, T cells in circulation will traffic to lymphoid and non-lymphoid organs in which they will either survive or undergo apoptosis. In order to systematically examine whether T cells traffic to these organs and persist within the tissue microenvironment, the mice were euthanized seven days after adoptive transfer, lymphoid and non-lymphoid organs of interest were isolated, and the sgRNA library representation in each organ sample was sequenced to assess which mutant T cells, relatively how many, and how frequently, survived in vivo. Collected and surveyed were: the liver, pancreas, lung, muscle and brain as representative non-lymphoid organs, as well as the spleen and several types of lymph nodes (LNs) as lymphoid organs (FIG. 1). The LNs collected were divided into three groups: skin draining lymph nodes (sLNs) that comprised of the inguinal, popliteal, axillary, and brachial lymph nodes; cervical lymph nodes (cLNs) that comprised of the 6 superficial lymph nodes; and abdominal lymph nodes (aLNs) that consisted of the mesenteric and the pancreatic lymph nodes (FIG. 1).

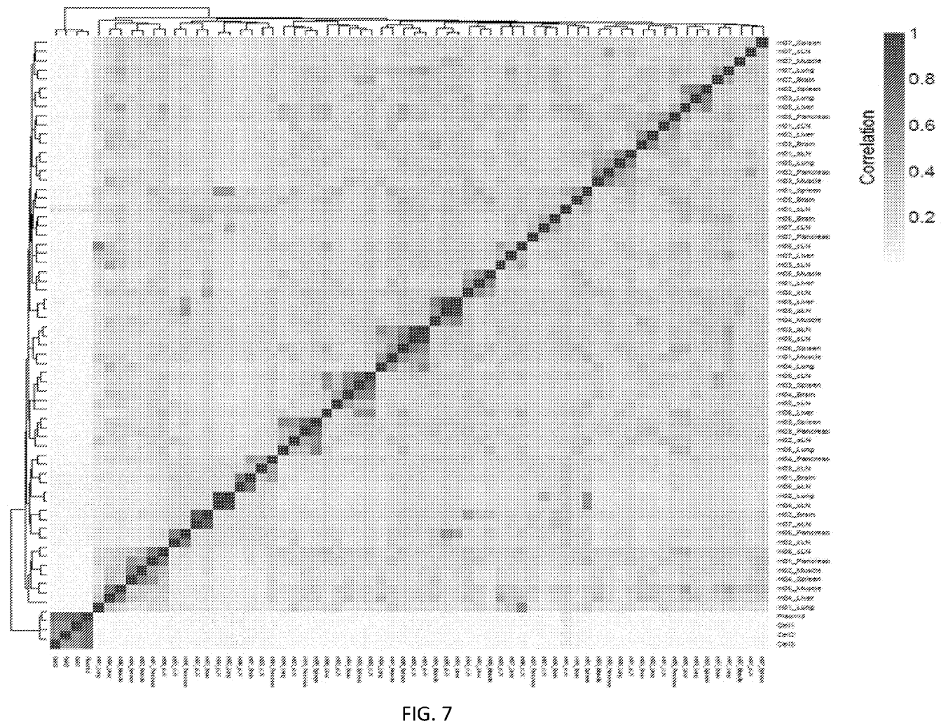

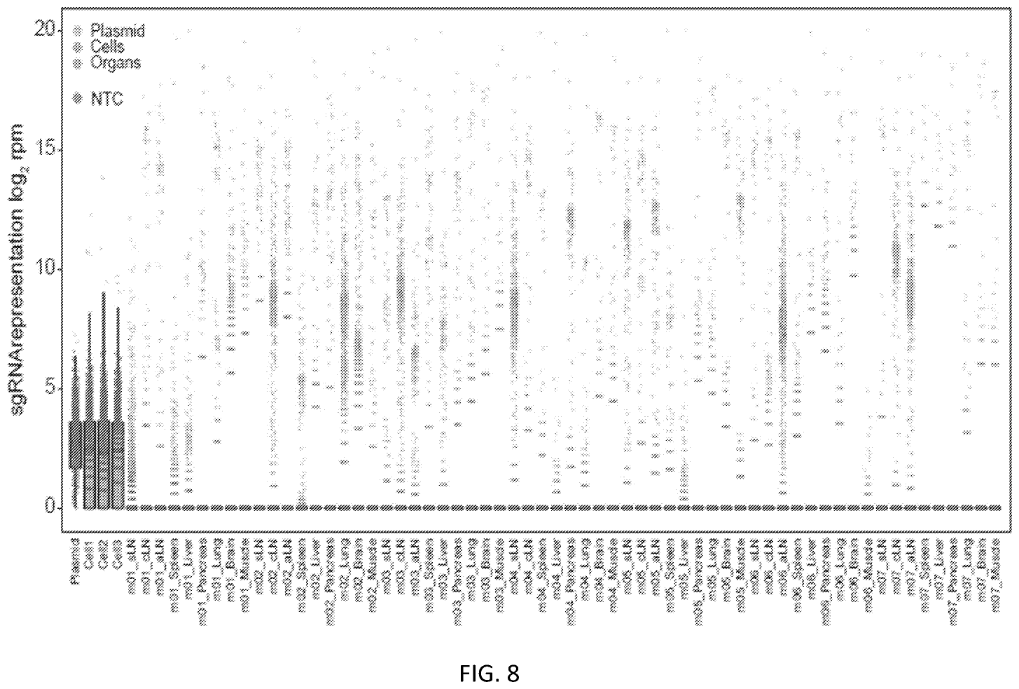

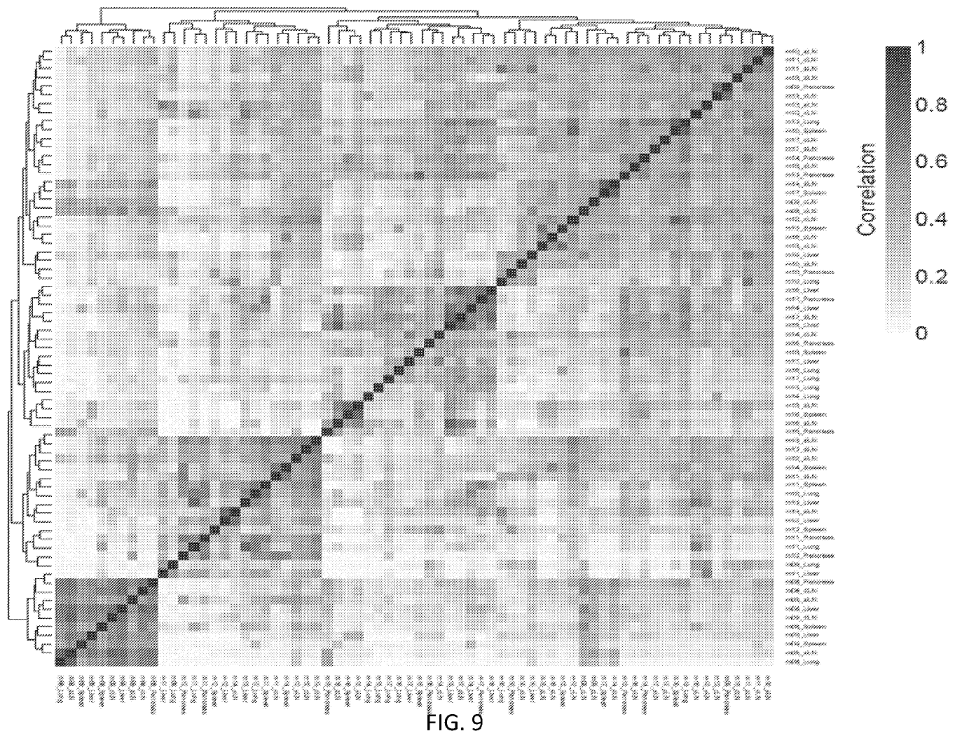

Illumina sequencing successfully read out the sgRNA library representation of the CD8⁺ T cells in all organs, as well as three representative pools of pre-injected MKO-transduced T cells. The library representation in all three replicates of uninjected T cells closely clustered with each other and the MKO plasmid library, whereas the library representation of all organs clustered together (FIG. 7). While the library representation of pre-injected T cells follows a log-normal distribution for both gene-targeting sgRNAs (GTS) and NTCs, the sgRNA representation in organs is characterized by the dominance of a small fraction of sgRNAs (FIG. 8), a signature of clonal expansion of a subset of targeted T cells. While an organ can be dominated by one or a few T cell mutants (e.g. a CD8⁺ T cell clone with an sgRNA targeting Program cell death protein 1 (PD-1/Pdcd1) dominated the aLN sample in mouse 3) (FIG. 1D), a given organ can also consist of multiple highly abundant, but non-dominating clones (FIG. 1D). Monoclonal (one major clone), oligoclonal (2 to 10 major clones each with ≥2% of total reads) and polyclonal (more than 10 clones with 2% or more reads) compositions of T cell variants exist in both lymphoid and non-lymphoid organs (FIG. 1D). These data revealed a global landscape of organ survival for mutant CD8⁺ T cells with a diverse TCR repertoire, and showed that a small subset of the variants from the MKO CD8⁺ T cell pool became highly enriched in vivo after trafficking and survival in a new host for 7 days.

The library representation within each sample was then analyzed to find enriched sgRNAs compared to the 1,000 NTC sgRNAs. To identify genes whose perturbation might result in enhanced ability of CD8⁺ T_{eff} cells to survive in differential organs in vivo, the sgRNAs and genes represented in the MKO library were ranked using multiple statistical metrics. At a false discovery rate (FDR) of 0.5% or lower, a set of significantly enriched sgRNAs were identified in each organ. Ranking sgRNAs by their prevalence (frequency of being enriched in an organ) (FIG. 1E) revealed dominant signatures of three types of genes: (1) immune genes (such as Lexm BC055111, Socs5, Zap70), consistent with their role in T cells; (2) genes regulating general cell growth and proliferation (e.g. tumor suppressor genes such as Tsc2, Nf1, Pten, and Trp53); as well as (3) genes with undocumented functions in CD8⁺ T cells or largely uncharacterized genes (such as Sgk3, Fam103a1, Phf21a, and 1110057K04Rik) (FIG. 1E). Ranking sgRNAs by the number of independent enriched sgRNAs also revealed these three types of genes, with the top three genes representing three different categories (Cd247—immune, Tsc2—growth, and Bpifb3—unknown) (FIG. 1F). In conjunction with a third criteria, in which a given sgRNA must comprise ≥2% of the reads in a single sample, a total of 11 genes were significantly enriched across all three criteria, again representing immune (Pdcd1, Cd247), growth (Apc, Nf1, Tsc2) and unknown (Csnk1a1, Fam103a1, Fam134b, Phf21a, Prkar1a, and Rab11b) genes (FIG. 1G). Pdcd1, also known as PD-1, is a well-established immune checkpoint regulator expressed on T cells (Ishida et al., 1992), and a major target of checkpoint blockade (Chen and Mellman, 2013). The fact that Pdcd1 passed all three criteria and emerged as a robust hit provided strong evidence for the validity of this approach. Many of the significantly enriched genes are membrane proteins involved in the immune system. Together, these data suggest that perturbation of these genes by CRISPR allows CD8⁺ T_{eff} cells to better survive in lymphoid and non-lymphoid organs in vivo.

### Example 2: Genome-Scale Screen for Trafficking and Survival with Effector CD8⁺ T Cells with Transgenic, Clonal TCR

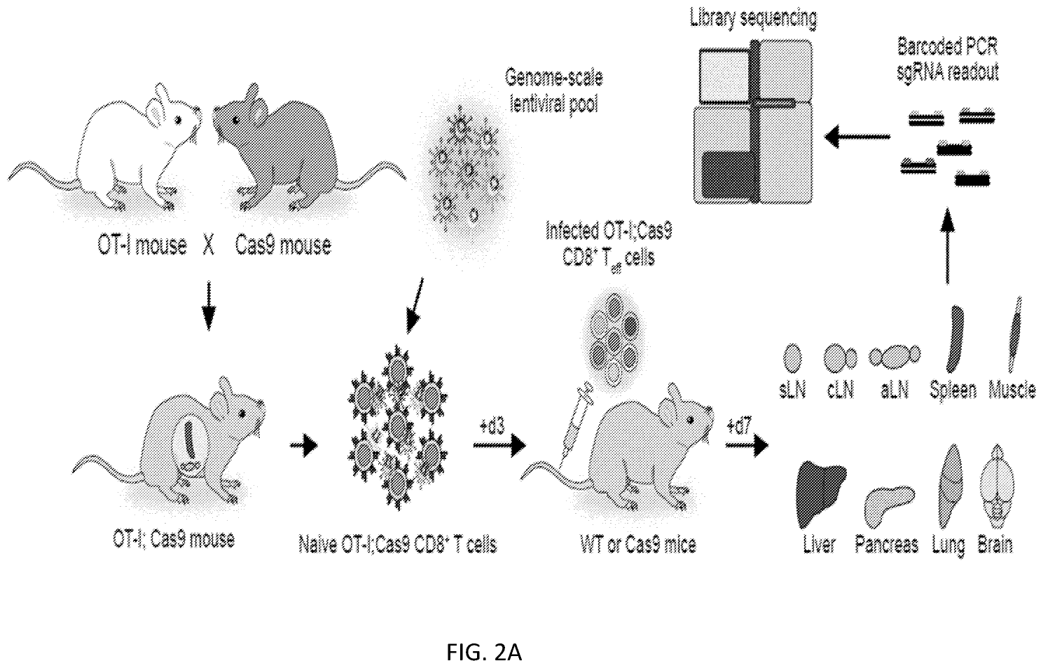

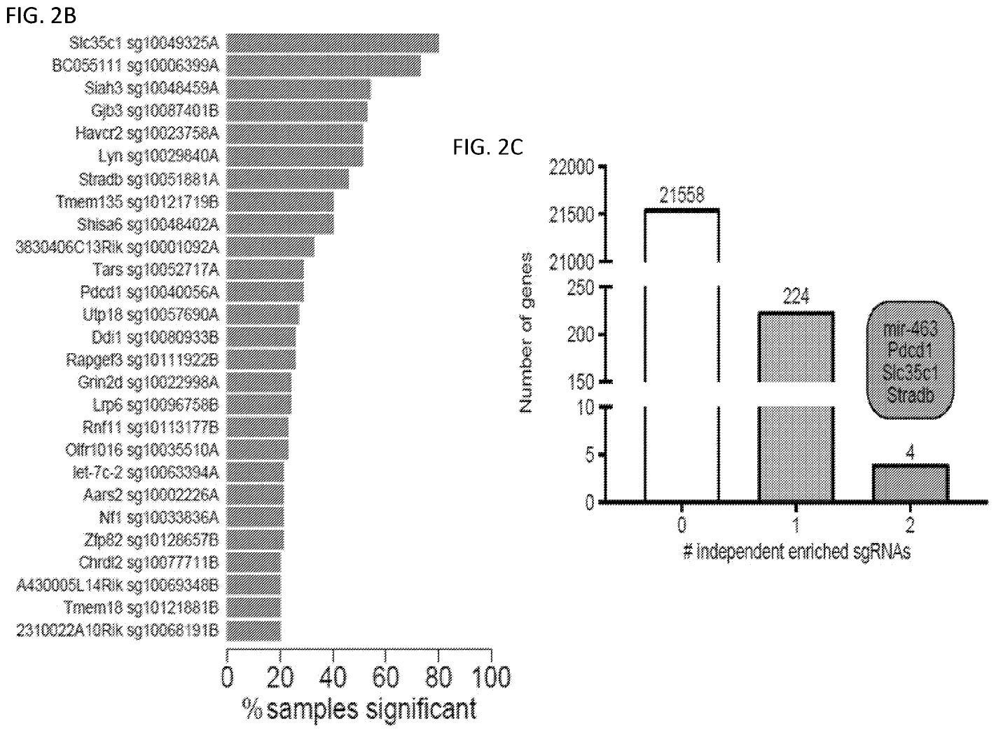

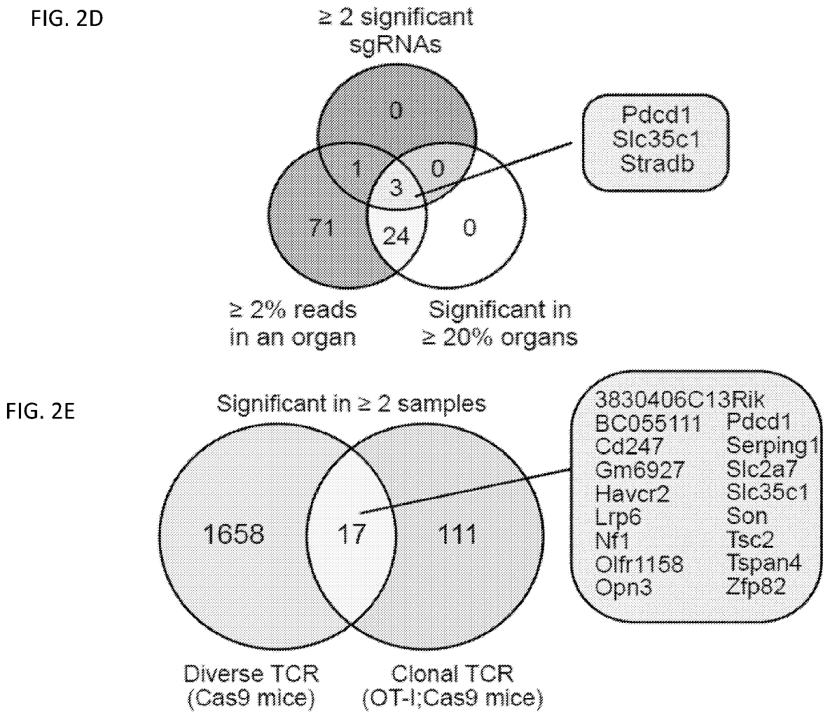

Due to the diversity of the TCR repertoire in Cas9 mice, certain genetic effects may be masked by the heterogeneity of the TCR pool. To address this issue and thereby provide a parallel picture in an isogenic setting, the genome-scale CRISPR screen was repeated with a homogenous pool of CD8⁺ T_{eff} cells that expressed the transgenic OT-I TCR, which specifically recognizes the SIINFEKL peptide of chicken ovalbumin (cOVA) presented on H-2K^{b}, a haplotype of MHC-I. Through genetic crosses, a mouse strain (OT-I; Cas9 mice) that expresses both Cas9 and the OT-I transgenic TCRs was generated (FIG. 2A). With these mice, the objective was to identify genes whose perturbation can result in enhanced ability of T_{eff} cells to survive in different organs in vivo following trafficking starting from clonal TCRs. Similarly, naive OT-I; Cas9 CD8⁺ T cells were isolated and mutagenized by transducing with the MKO lentiviral library with 3 infection replicates. Then they were adoptively transferred into wildtype B6 (n=5) or Cas9 (n=5) recipient mice (n=10 total) (FIG. 2A). Seven days post adoptive transfer, the mice were euthanized, relevant lymphoid and non-lymphoid organs collected, and then Illumina sequencing was performed to readout the sgRNA library representation. The sgRNA library representation revealed a global landscape of organ survival for mutant T_{eff} cells with clonal TCR in vivo.

To identify genes modulating trafficking and survival of OT-I; Cas9 CD8⁺ T_{eff} cells, sgRNAs and genes represented in the MKO library were ranked using multiple statistical metrics. Ranking sgRNAs by their prevalence (frequency of being enriched in an organ) (FIG. 2B) again revealed dominant signatures of three types of genes: (1) immune genes (e.g. BC055111, Hacvr2, Lyn and Pdcd1); (2) growth regulators (e.g. Nf1), although fewer compared to the previous screen; as well as (3) genes with undocumented functions in CD8⁺ T cells or largely uncharacterized genes (e.g. Slc35c1, Siah3, Gjb3, Tmem135 and Shisa6) (FIG. 2B). Havcr2, also known as Tim-3, is a well-established immune checkpoint regulator expressed on T cells (Chen and Flies, 2013), and currently an active target for immunomodulation (Sakuishi et al., 2010). Ranking sgRNAs by the number of independent enriched sgRNAs revealed 4 genes with multiple enriched sgRNAs (mir-463, Pdcd1, Slc35c1, and Stradb) (FIG. 2C). In conjunction with the sgRNA abundance criteria (≥2% of total reads in a sample), a total of 3 genes were significantly enriched across all three criteria (Pdcd1, Slc35c1, and Stradb) (FIG. 2D). These data together suggest that the CRISPR targeting of these genes allows TCR-clonal OT-I; CD8⁺ T_{eff} cells to better survive in lymphoid and non-lymphoid organs in vivo.

To find which candidate genes can modulate T cell function in both diverse (Cas9 CD8⁺ T cells) and clonal TCR (OT-I; Cas9 CD8⁺ T cells), the gene sets from these two screens were directly compared. A total of 17 genes were identified in both screen as common hits, which again included immune genes (BC055111, Cd247, Hacvr2, and Pdcd1), tumor suppressors (Nf1 and Tsc2), and unknown or uncharacterized genes in T cells (e.g. Gm6927, Slc35c1, Slc2a7, Lrp6, and Zfp82). The emergence of multiple immune genes as mutual top hits, in both diverse TCR and clonal TCR settings, further validated the rigor of this approach, gaining higher confidence for the phenotypes of the unknown genes or those previously not associated with T cell function.

### Example 3: In Vivo Genome-Scale Screen of TCR-Engineered T_{eff} Cells Infiltrating Tumors Expressing a Model Antigen

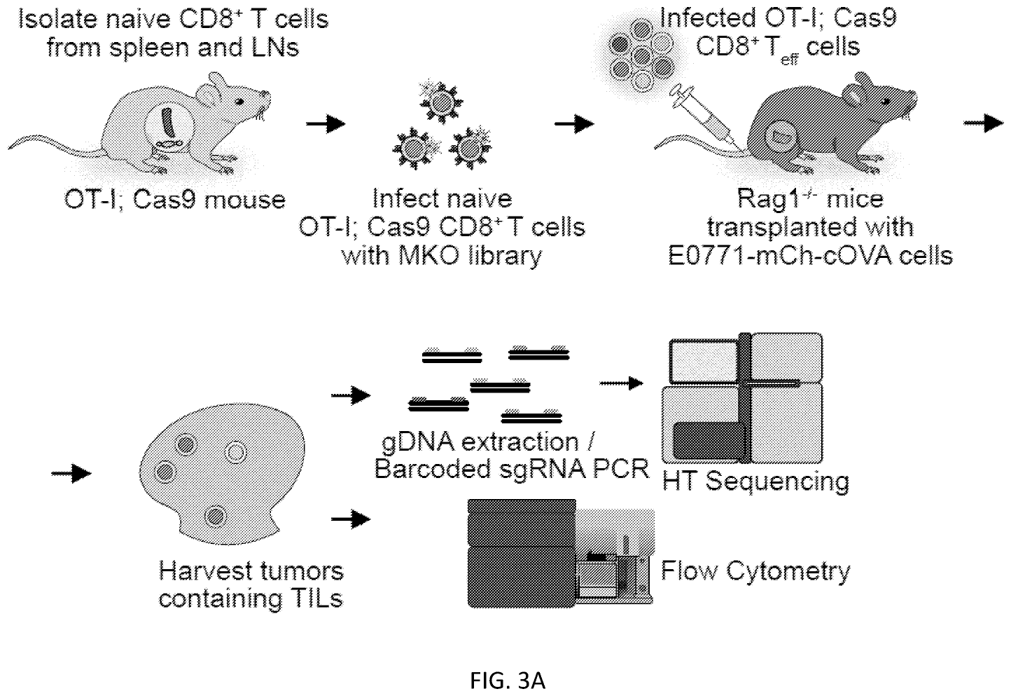

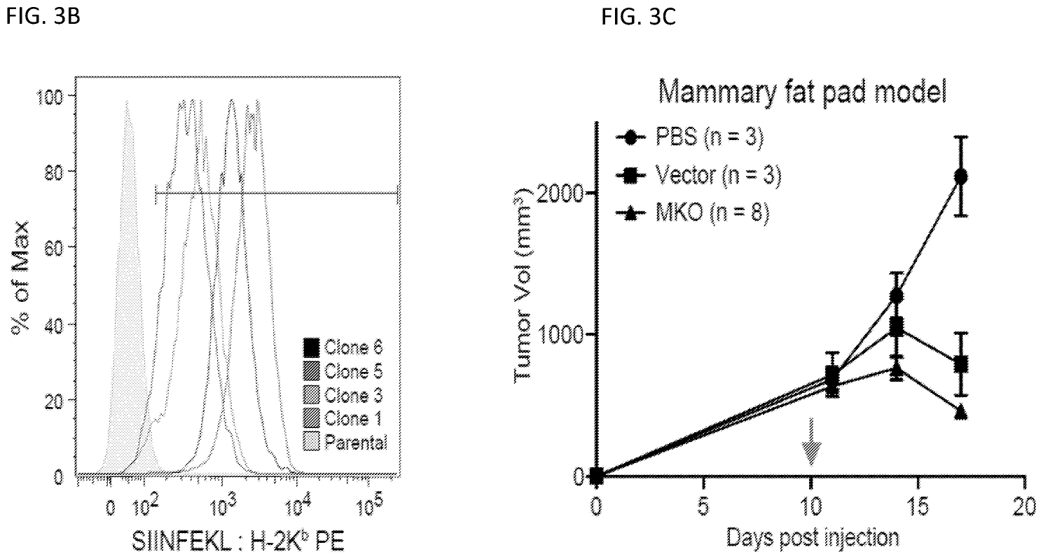

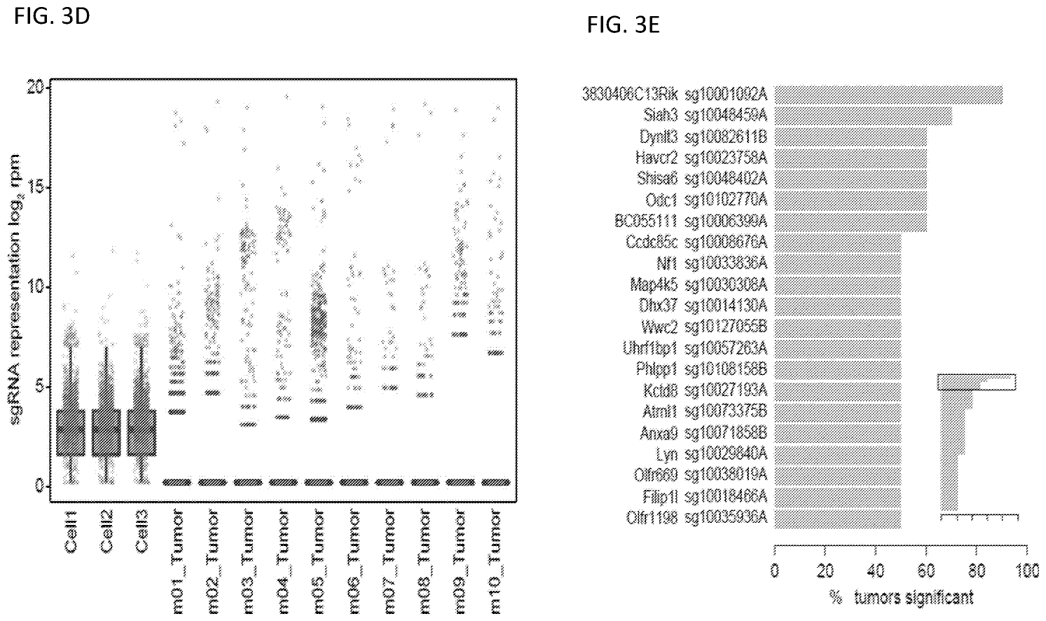

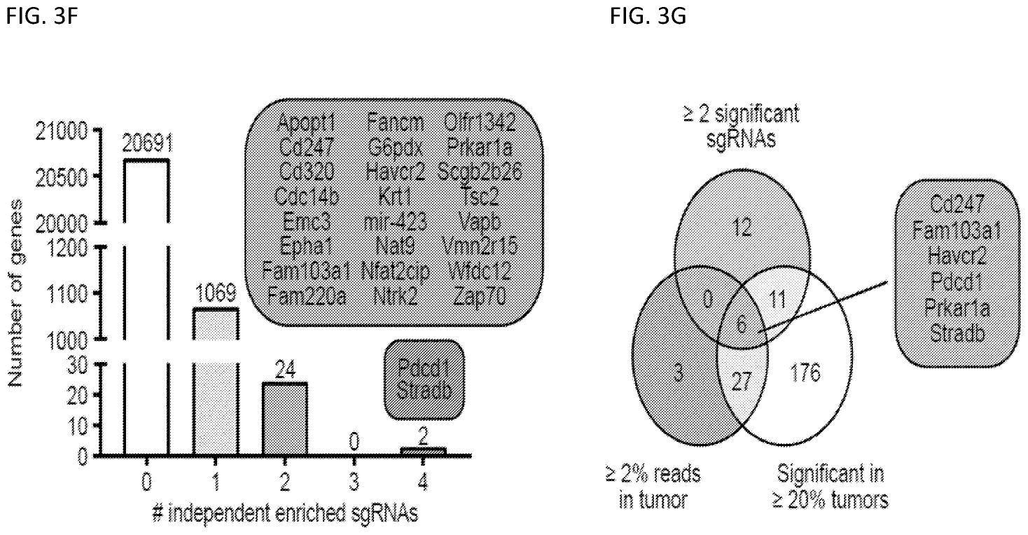

After establishing these robust experimental and statistical methodologies, T cell CRISPR screens were performed in immunotherapy settings. To enable T cell recognition of cognate antigen in cancer cells, several clonal cell lines that constitutively express cOVA were generated (FIG. 3A). Using a well-established antibody that recognizes the SIINFEKL: H-2K^{b} complex, it was confirmed that the SIINFEKL peptide was presented on surface H-2K^{b} (FIG. 3B). Clone 3 of E0771-mCherry-cOVA (E0771-mCh-cOVA for short) cell line was chosen for further in vivo studies because it presented a lower level of SIINFEKL peptide on H-2K^{b} (FIG. 3B), thereby enhancing the sensitivity of the screen in order to better detect genes with phenotypes. Despite expressing lower levels of presented SIINFEKL, Clone 5 was not chosen due to its putative bi-modal presentation of SIINFEKL: H-2K^{b} (FIG. 3B). Transplantation of 5×10⁶ clone 3 cells into Rag1^{-/-} mice led to rapid tumor formation in 10 days (FIG. 3C-3D).

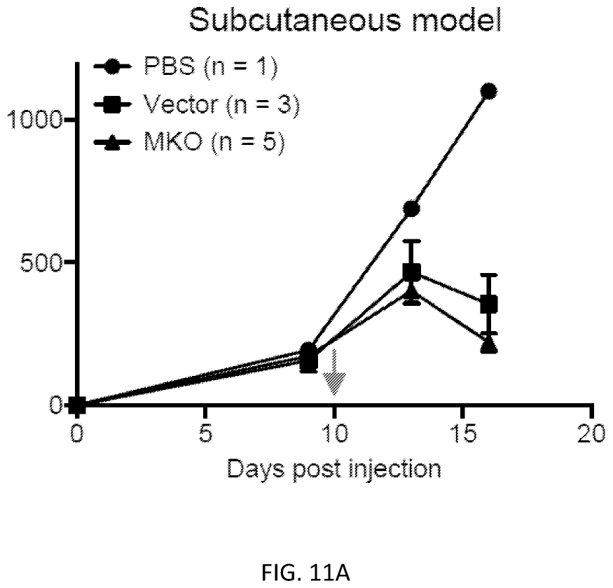

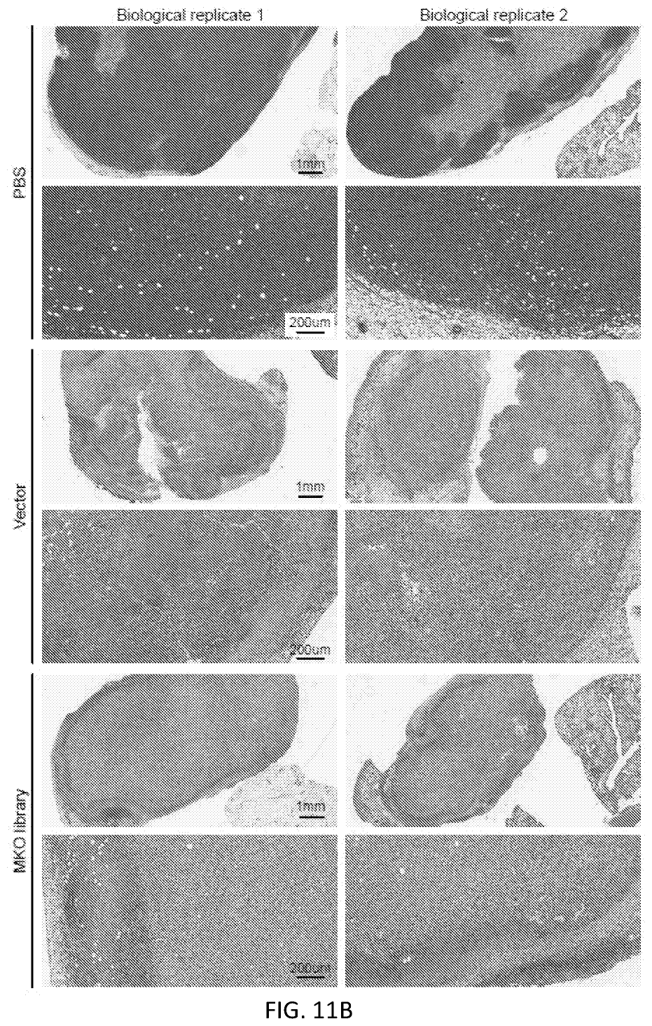

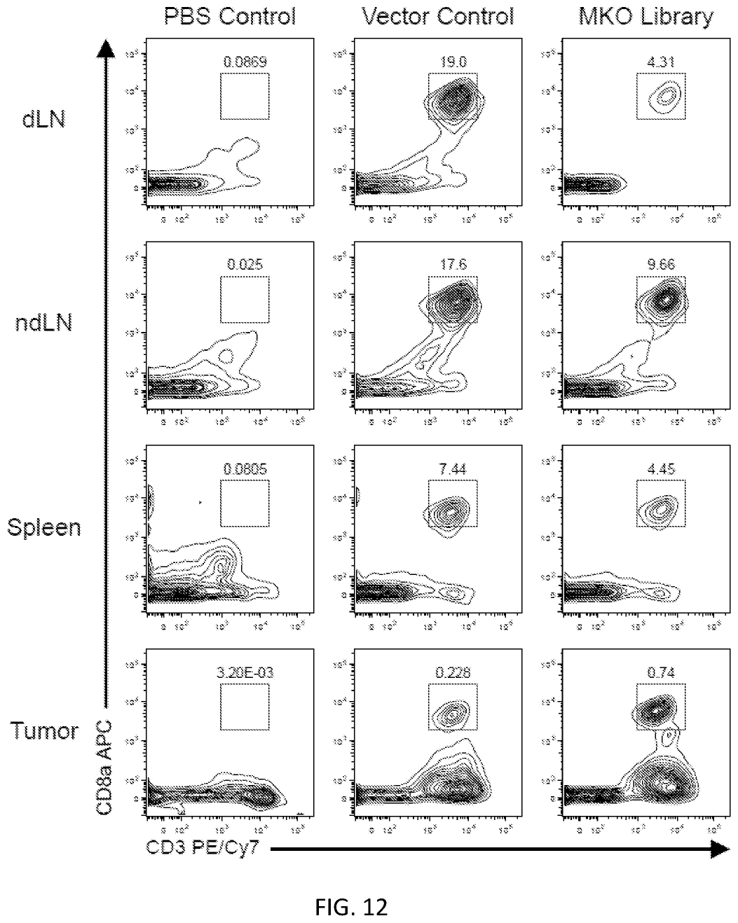

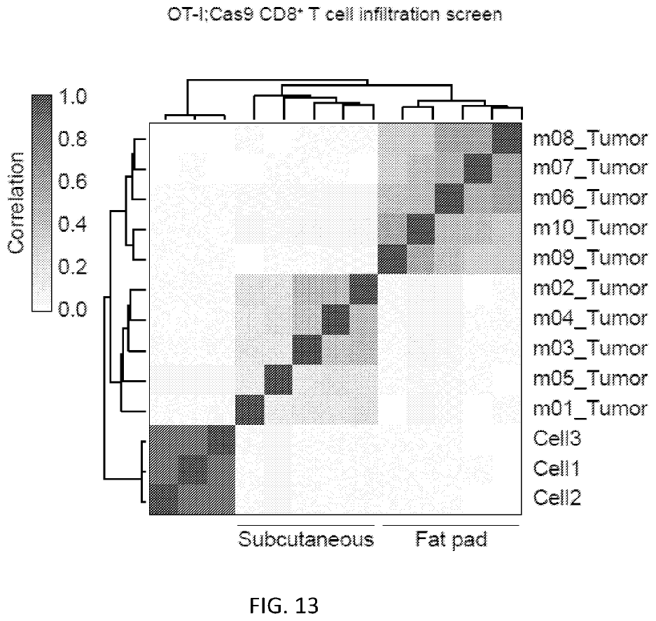

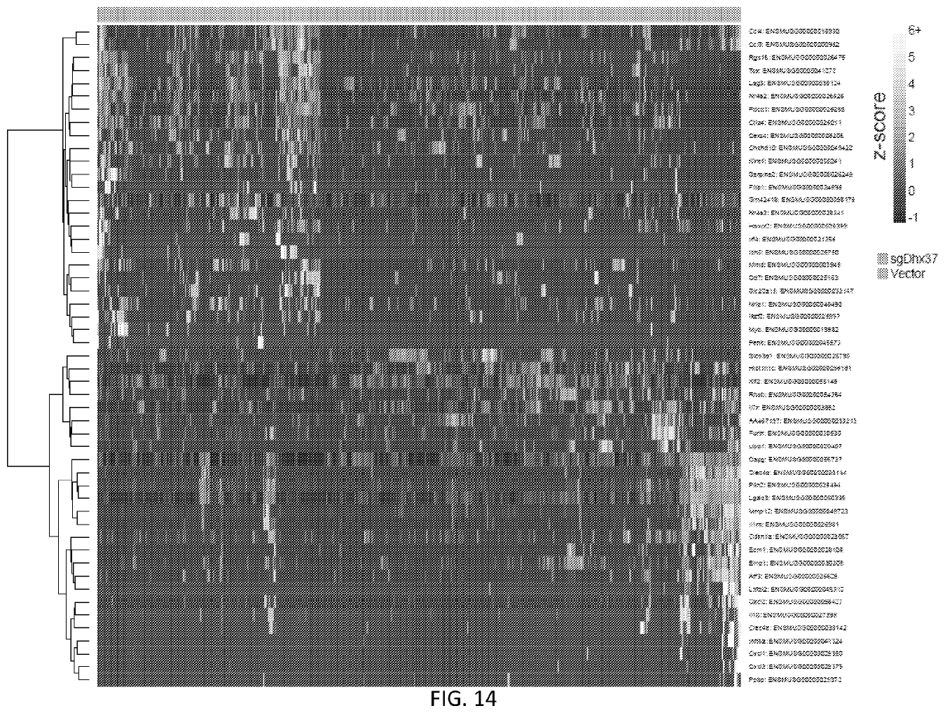

Naive CD8⁺ T cells from OT-I; Cas9 mice were isolated, mutagenized with the MKO sgRNA library, and 1×10⁷ cells adoptively transferred into Rag1^{-/-} mice bearing cOVA-expressing tumors grown from E0771-mCherry-cOVA clone 3 cells (FIG. 3A). Tumor size was measured throughout the experiment. T cell injections (either vector or MKO transduced) mitigated tumor growth, in sharp contrast with PBS (Endpoint tumor size vector vs PBS, unpaired two-sided t-test, p=0.02; MKO vs PBS, p<0.0001) (FIG. 3C). The MKO mutagenized population had a stronger therapeutic effect compared to vector controls (Endpoint tumor size MKO vs vector, unpaired two-sided t-test, p=0.03) (FIG. 3C). This anti-tumor effect also holds in a subcutaneous transplant model, although to a less extent (FIG. 11A). Seven days post-adoptive transfer (17 days after cancer cell transplantation), the mice were euthanized and the tumors isolated for analysis of tumor-infiltrating lymphocytes (TILs). Histological and pathological analysis revealed the existence of lymphocytes in the tumors from mice injected with vector and MKO CD8⁺ T_{eff} cells, but not in tumors of PBS treated mice (FIG. 11B). Flow cytometric analysis (n=3 mice) of single-cell suspensions of organs and tumors detected a large number of CD8+T_{eff} cells in Rag1^{-/-} mice receiving T cell injections but not those receiving PBS (FIG. 12), indicating that the CD8⁺ T_{eff} cells present in these samples were adoptively transferred. Representative tumors from a parallel cohort of mice (n=10) were subjected to high-throughput sgRNA library sequencing (FIG. 3A), which revealed the sgRNA representations of MKO mutagenized OT-I; Cas9 CD8⁺ T_{eff} cells before injection and in all tumor samples (FIG. 3D, FIG. 13).

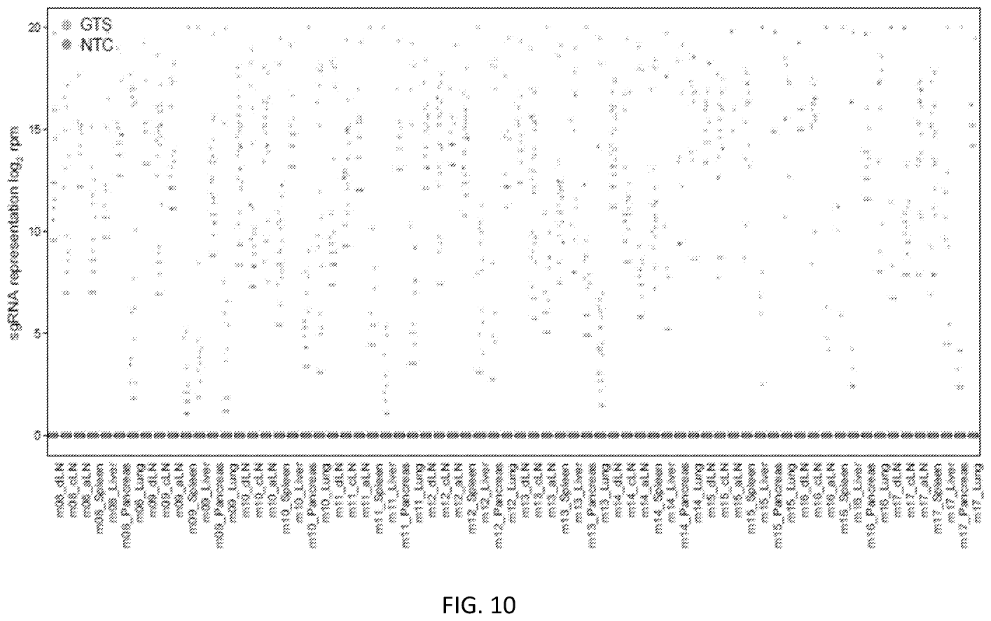

Using the same criteria as described previously herein (FDR<0.5%), significantly enriched sgRNAs in each tumor were identified (FIG. 3E, FIG. 10). Ranking sgRNAs by their prevalence across tumors again revealed dominant signatures of immune genes (such as Tim3 Havcr2, BC055111, and Lyn), growth genes (e.g. Nf1), as well as genes with undocumented function in CD8⁺ T cells or generally uncharacterized genes (such as Shisa6, Siah3, Odc1, Dhx37, and 3830406C13Rik) (FIG. 3E). Ranking sgRNAs by the number of independent enriched sgRNAs revealed 26 genes with multiple enriched sgRNAs (FIG. 3F). Notably, two genes (Pdcd1 and Stradb) had 4 enriched sgRNAs, representing independent evidence for their phenotypes (FIG. 3F). After considering a third criteria of sgRNA abundance (≥2% of total reads in a single tumor) representing substantial TIL clones, a total of 6 genes were significantly enriched across all three criteria (Cd247, Fam103a1, Hacvr2, Pdcd1, Prkar1a, and Stradb) (FIG. 3G). These data together suggested that the loss-of-function of these genes make CD8⁺ T_{eff} cells consistently better in terms of tumor infiltration and survival in the tumor microenvironment.

### Example 4: High-Throughput Identification of Genes Modulating Effector CD8+ T Cell Degranulation Upon Encountering Tumor Antigen

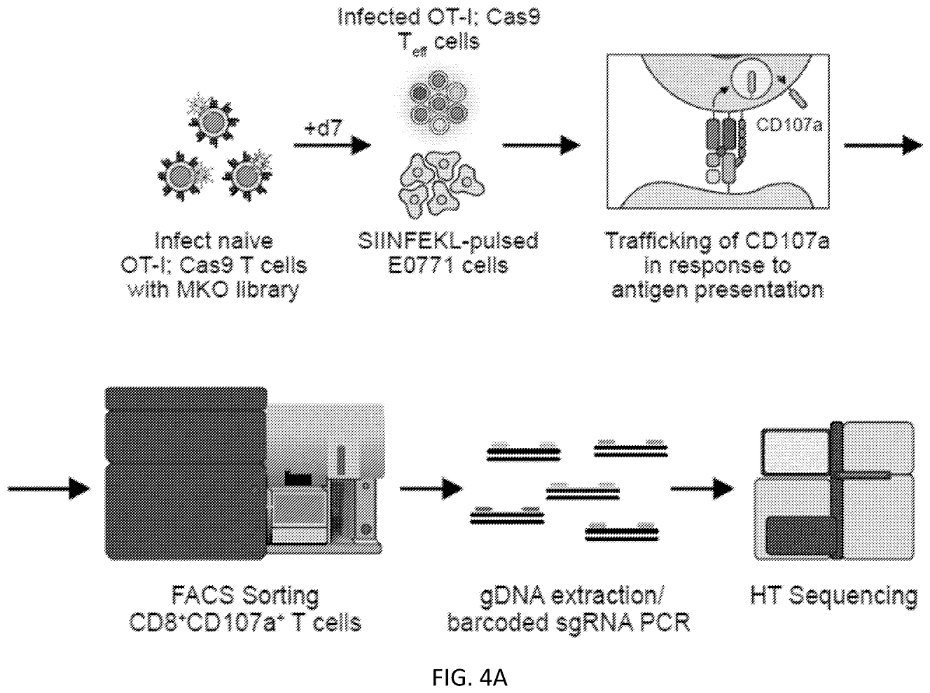

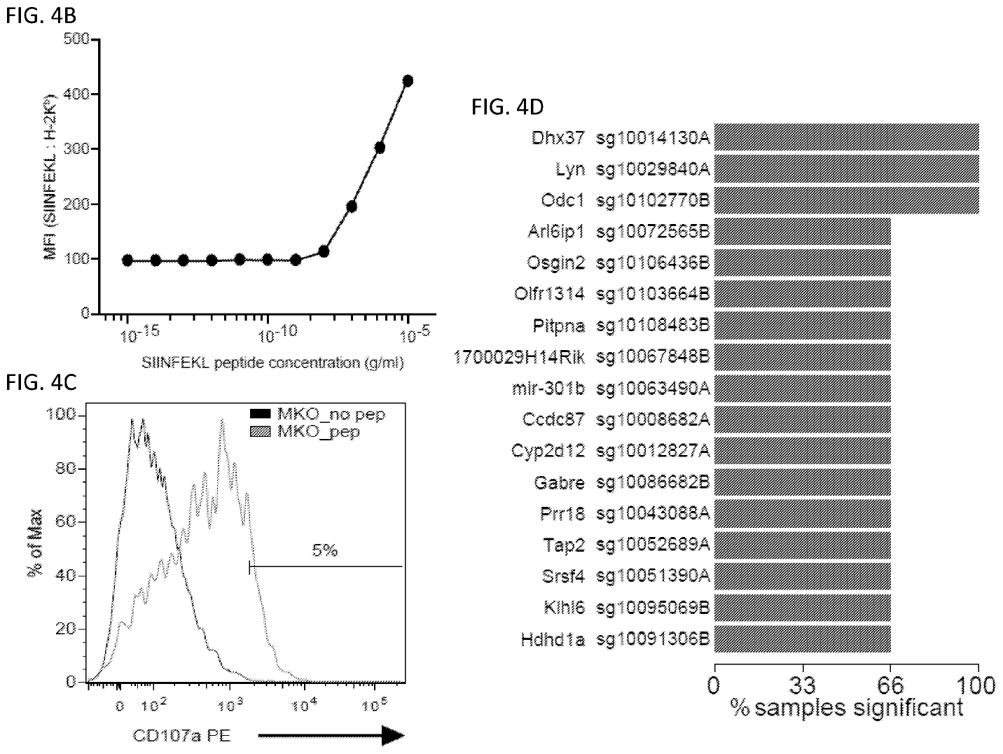

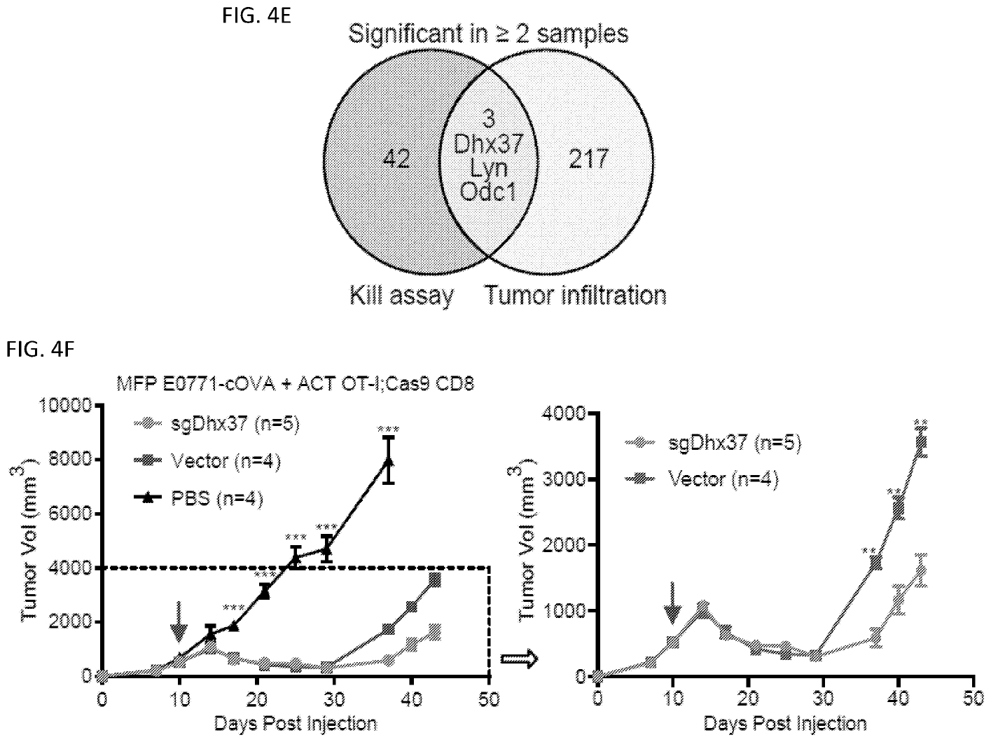

Having observed an anti-tumor effect in vivo, subsequent experiments set out to identify genes that could modulate the ability of CD8⁺ T_{eff} cells to target and kill cancer cells bearing tumor-specific antigen. A degranulation screen was developed using a co-culture system in which OT-I; Cas9 CD8⁺ T_{eff} cells would degranulate in response to E0771 cancer cells presenting SIINFEKL peptide (FIG. 4A). E0771 cells were pulsed with varying concentrations of SIINFEKL peptide, and found to present SIINFEKL peptide on surface MHC-I in a dose-dependent manner (FIG. 4B). To perform a high-throughput CRISPR degranulation screen, naive OT-I; Cas9 CD8⁺ T cells were isolated and transduced with MKO library. The cells were incubated in cRPMI supplemented with IL-2, IL-12, anti-CD28 and anti-CD3 for stimulation for 6 days, rested for 12 hours prior to the experiment on untreated plates, and then the mutagenized CD8⁺ T_{eff} cells were co-cultured with SIINFEKL-pulsed E0771 cells at 1:1 (T cell: cancer cell) ratio. T cells were incubated in media containing anti-CD107a antibody to label the transient deposition of surface CD107a, a marker of T cell granules that is temporarily presented on the cell surface when T cells encounter cognate antigen on MHC. A total of 1×10⁷ T cells per replicate with three biological replicates were analyzed. The top 5% CD107a⁺ cells (FIG. 4C) were sorted then subjected to genomic DNA extraction, CRISPR library readout, and screen data analysis (FIG. 4A). Using the FDR<0.5% significance cutoff, significantly enriched sgRNAs in sorted CD8⁺CD107a⁺ T cells after exposure to SIINFEKL-pulsed E0771 tumor cells in co-culture were identified (FIG. 4D). Remarkably, three genes were significantly enriched in all three samples (Dhx37, Lyn, and Odc1), and they were also found to be significant in the tumor infiltration screen (FIG. 4E). These data together pinpointed Dhx37, Lyn, and Odc1 as promising targets for potentially augmenting anti-tumor activity in vivo by CD8⁺ T cells.

### Example 5: Enhanced Anti-Tumor Function and Single-Cell Transcriptomic Signatures of OT-I; Cas9 CD8+T_{eff} Cells with Dhx37 Perturbation

The phenotype of Dhx37 was examined in a model of immunotherapy. Two sgRNAs targeting Dhx37 were cloned into the T cell CRISPR vector, and virus prep and T cell infection were performed as described above. 5×10⁶ sg-Dhx37 or vector lentivirus transduced OT-I; Cas9 CD8⁺ T cells were adoptively transferred into mice bearing breast tumors, 10 days post mammary fatpad transplantation of 5×10⁶ clone 3 mCh⁺cOVA⁺E0771 cells. Again, a 1:1 (T cell: cancer cell) ratio was adopted at the time of their respective injections (of note, the cancer cells in a day-10 tumor might largely outnumber 5×10⁶ T cells). Despite initially growing for 3-days post adoptive transfer, the tumors regressed in the ensuing 2.5 weeks (FIG. 4F, left panel). Both vector and sgDhx37 infected OT-I; Cas9 CD8+T_{eff} cells demonstrated strong anti-tumor effects beginning 7 days after adoptive transfer (Vector or sgDhx37 vs PBS, two-sided t-test, adjusted p<0.001 from d17 onwards (Benjamini, Krieger and Yekutieli method)) (FIG. 4F, left panel). As a result, sgDhx37 infected OT-I; Cas9 CD8⁺ T_{eff} cells (n=5 mice) significantly suppressed the relapse when compared to mice treated with vector-infected OT-I; Cas9 CD8⁺ T_{eff} T cells (n=4 mice) (two-sided t-test, adjusted p<0.01 from d37 onwards) (FIG. 4F, right panel). These data demonstrated that targeting Dhx37 with CRISPR/Cas9 and sgRNAs enhanced the anti-tumor effects of OT-I; Cas9 CD8⁺ T_{eff} cells against E0771 tumors expressing cognate antigen cOVA.

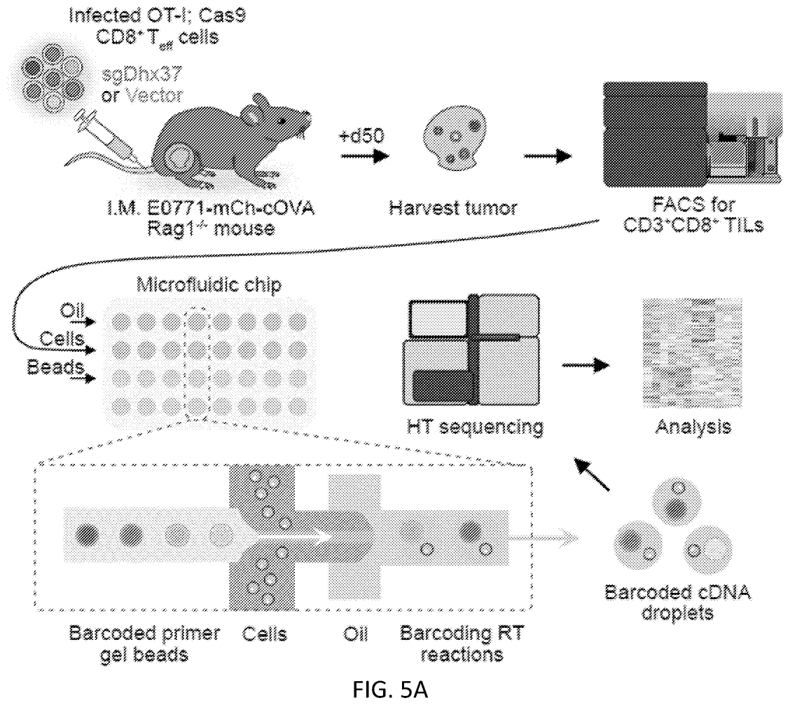

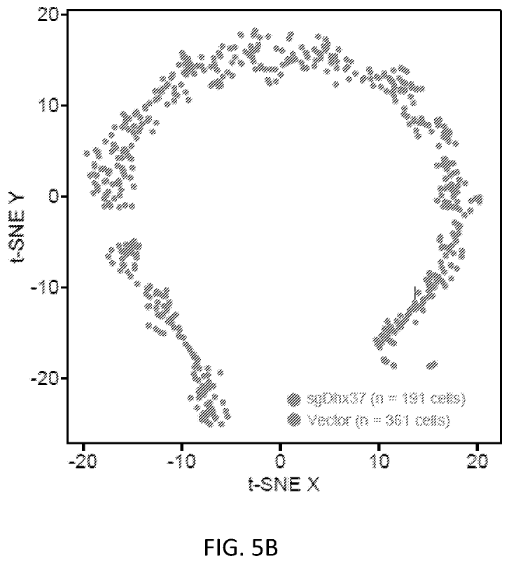

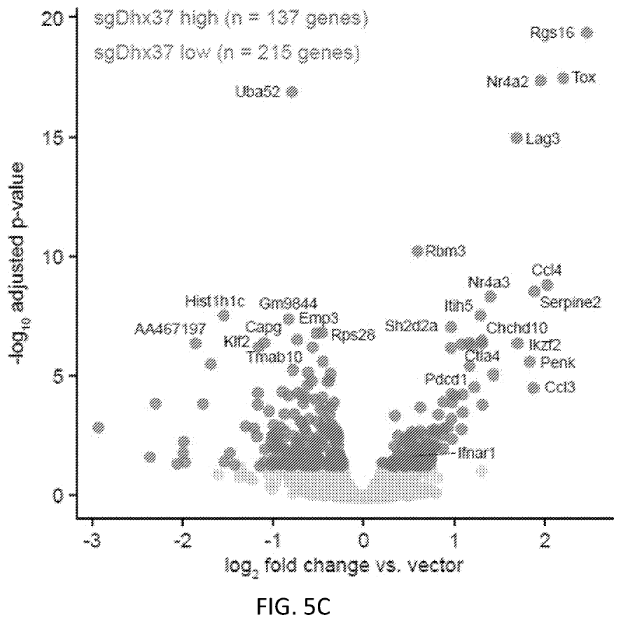

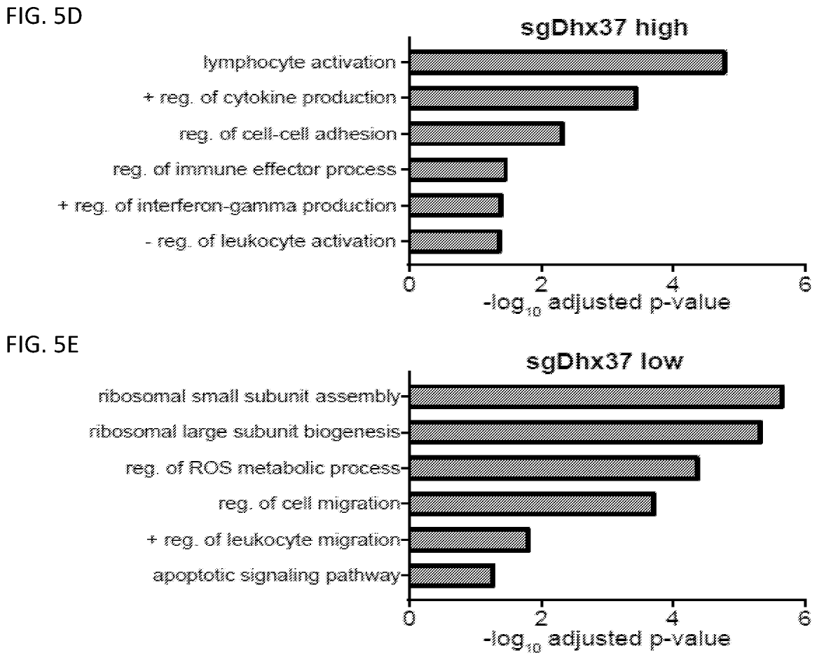

Dhx37 is a DEAH box RNA helicase reported to regulate escape behavior via glycine receptor expression in zebrafish, but has not been previously associated with T cell function in mammalian species. The putative ATP-Dependent RNA Helicase domain and conservation implies that it might affect gene expression and cellular function. To investigate the effect of gene expression alteration upon Dhx37 perturbation, transcriptome analysis of sgDhx37 OT-I; Cas9 CD8⁺ T cells in the form of TILs was performed. Because TILs are in the heterogeneous tumor microenvironment, which might influence the state of TILs leading to highly variable gene expression, single cell RNA-seq (scRNAseq) was used to investigate the transcriptomes of sgDhx37 TILs. Tumor-bearing mice were euthanized and single-cell suspensions generated from tumors by physical dissociation and enzymatic digestion. TILs were collected by staining and sorting the live CD3⁺CD8⁺ cells with FACS. Because TILs only consisted of a tiny fraction of cells in these tumors, the vast majority of single cell suspensions were sorted from whole tumors, and 3×10³ to 2×10⁴ live CD3⁺CD8⁺ TILs were collected per tumor (FIG. 5A). These freshly collected TILs were subjected to an emulsion-based microfluidic device to barcode the CD8⁺ TILs from sgDhx37 and vector groups, and scRNAseq library preparation was performed. The library was sequenced with Illumina Hiseq platform for unique molecular identifiers (UMIs), cellular barcodes, and the transcriptome in each cell was quantified.

After processing, stringent filtering, and normalizing the raw scRNA-seq data, the final dataset was comprised of 552 cells (sgDhx37, n=191 cells; vector, n=361 cells), measuring a total of 8,244 expressed genes in TILs. t-SNE dimensional reduction was first performed to visualize the overall transcriptomic landscape of these cells (FIG. 5B). From this global view, sgDhx37 and vector-treated TILs spanned a continuum of transcriptomic states, indicating a degree of heterogeneity among the TIL population. Differential expression analysis was subsequently performed between sgDhx37 and vector treated TILs, identifying sets of significantly upregulated and downregulated genes. 215 genes were significantly downregulated in sgDhx37 TILs, while 137 genes were significantly upregulated (Benjamini-Hochberg adjusted p<0.05), with the mostly highly upregulated genes as Rgs16, Tox and Nr4a2 (FIG. 5C). Rgs16 was found as an IL-2-dependent activation gene in human T lymphocytes, and is enriched in activated/effector T cells. Nr4a2 is a nuclear receptor essential for thymic regulatory T cell (T_{reg}) development and homeostasis, and associated with T cell activation, although its specific function in CD8⁺ T cell or TILs is not well characterized. Tox encodes a HMG box protein involved in both CD8+ and CD4⁺ T cell development, to some degree without the requirement of MHC-TCR interactions. Other significantly upregulated genes included known immune-related genes such as Eomes, Nr4a3, Lag3, Ccl4, Ifnar1, and Ikzf2, as well as genes with less knowledge in CD8⁺ T cells or TILs (FIG. 5C). Collectively as a gene set, gene ontology analysis revealed multiple immune-related pathways that were significantly upregulated in sgDhx37 TILs (adjusted p<0.05), including lymphocyte activation, positive regulation of cytokine production, regulation of cell-cell adhesion, regulation of immune effector process, and positive regulation of interferon-gamma production (FIG. 5D). Somewhat intriguingly, sgDhx37 upregulated genes also include genes involved in negative regulation of leukocyte activation such as Ctla4 and Pdcd1, albeit to a lesser extent (approximately 2-fold change), although these genes might have multifaceted roles in a delicate network of immune gene regulation. Taken together, the scRNAseq data revealed significant changes in the transcriptomes of sgDhx37 TILs in the heterogeneous tumor microenvironment at the single-cell level.

### Example 6

Herein, genome editing was coupled to high-throughput screening approaches, and directly applied to systematically study the trafficking and survival of CD8⁺ T cells in vivo, both in physiological and pathological (cancer) settings. These screens generated large-scale maps of genetic factors modulating the trafficking, survival and tumor infiltration of CD8⁺ T cells, and identified enriched genes belonging to various functional categories including those not documented in literature. Further validation of Dhx37 demonstrated a case that modulation of these hits can lead to enhanced anti-tumor activity in vivo. Single-cell transcriptomic interrogation of sgDhx37 TILs revealed distinct alterations in immune genes signatures. While the current study focused on CD8⁺ T cells, this approach can readily be applied to study other type of T cells such as CD4⁺ T helper cells or T_{regs}. Although the immunotherapy model in this study was based on orthotopic transplantation of breast cancer cells, a variety of cancer models such as genetically engineered mouse models and genome-editing based cancer models for diverse cancer types are all possible alternatives. Utilization of this approach will advance the understanding of genetic control of T cells against cancer, which will have direct implications on CAR-T, checkpoint blockade, or other forms of immunotherapies.

In summary, CD8⁺ T cells play fundamental roles in the adaptive immune response mounted against intracellular pathogens and tumors, with a central role in the cancer-immune response. Due to the complexity of immunological networks, the highly dynamic tumor microenvironment, and the delicate interplay of cancer cells and immune cells, there may be other important mechanisms and potential therapeutic targets outside of checkpoint inhibitors. The present study demonstrates a proof-of-principle and provides a platform for unbiased discovery in CD8⁺ T cells. This study serves as an early stage reference for high-throughput genetic interrogation of immune cells in vivo, which can be broadly applied for diverse studies in immunology and immunotherapy.

### OTHER EMBODIMENTS

The recitation of a listing of elements in any definition of a variable herein includes definitions of that variable as any single element or combination (or subcombination) of listed elements. The recitation of an embodiment herein includes that embodiment as any single embodiment or in combination with any other embodiments or portions thereof.

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

1. A method of enhancing T cell based immunotherapy in a subject, the method comprising administering to the subject in need thereof a genetically modified T cell wherein a gene selected from the group consisting of Dhx37, Lyn, Slc35c1, Lexm, Fam103a1 and Odc1 has been mutated in the T cell.

2. The method of claim 1, wherein the T cell is selected from the group consisting of a CD8+, a CD4+, a T regulatory (Treg) cell and a Chimeric Antigen Receptor (CAR)-T cell.

3. The method of claim 1, wherein the subject is a human.

4. The method of claim 1, wherein at least one additional gene has been mutated in the T cell.

5. The method of claim 4, wherein the at least one additional gene is selected from the group consisting of Dhx37, Lyn, Slc35c1, Lexm, Fam103a1 and Odc1.

6. The method of claim 1, further comprising administering an additional treatment to the subject.

7. The method of claim 6, wherein the additional treatment is selected from the group consisting of an immune checkpoint inhibitor, a PD-1 inhibitor, and a CTLA-4 inhibitor.

8. A method of performing adoptive cell transfer therapy in a subject, the method comprising administering to the subject in need thereof a genetically modified T cell, wherein a gene selected from the group consisting of Dhx37, Lyn, Slc35c1, Lexm, Fam103a1 and Odc1 has been mutated in the T cell.

9. The method of claim 8, wherein the T cell is selected from the group consisting of a CD8+, a CD4+, a T regulatory (Treg) cell, and a CAR-T cell.

10. The method of claim 8, wherein the subject is a human.

11. The method of claim 8, wherein at least one additional gene has been mutated in the T cell.

12. The method of claim 11, wherein the at least one additional gene is selected from the group consisting of Dhx37, Lyn, Slc35c1, Lexm, Fam103a1 and Odc1.

13. The method of claim 8, further comprising administering an additional treatment to the subject.

14. The method of claim 13, wherein the additional treatment is selected from the group consisting of an immune checkpoint inhibitor, a PD-1 inhibitor, and a CTLA-4 inhibitor.

15. A method of treating cancer in a subject in need thereof, the method comprising administering to the subject a genetically modified T cell wherein a gene selected from the group consisting of Dhx37, Lyn, Slc35c1, Lexm, Fam103a1 and Odc1 has been mutated in the T cell.

16. The method of claim 15, wherein the T cell is selected from the group consisting of a CD8+, a CD4+, a T regulatory (Treg) cell, and a CAR-T cell.

17. The method of claim 15, wherein the subject is a human.

18. The method of claim 15, wherein at least one additional gene has been mutated in the T cell.

19. The method of claim 18, wherein the at least one additional gene is selected from the group consisting of Dhx37, Lyn, Slc35c1, Lexm, Fam103a1 and Odc1.

20. The method of claim 15, further comprising administering an additional treatment to the subject.

21. The method of claim 20, wherein the additional treatment is selected from the group consisting of an immune checkpoint inhibitor, a PD-1 inhibitor, and a CTLA-4 inhibitor.

22. A method of treating cancer in a subject in need thereof, the method comprising administering to the subject a therapeutically effective amount of an inhibitor of Dhx37.

23. The method of claim 22, wherein the inhibitor is selected from the group consisting of an antibody, an siRNA, and a CRISPR system.

24. The method of claim 23, wherein the CRISPR system comprises a Cas9, and at least one sgRNA complementary to Dhx37.

25. The method of claim 24, wherein the sgRNA comprises a nucleotide sequence selected from the group consisting of SEQ ID NOs: 1-10.

26. The method of claim 24, wherein the sgRNA comprises a nucleotide sequence selected from the group consisting of SEQ ID NOs: 11-820.

27. The method of claim 23, wherein the antibody recognizes and binds to at least one amino acid sequence selected from the group consisting of SEQ ID NOs: 3022-3031.

28. The method of claim 22, further comprising administering an additional treatment to the subject.

29. The method of claim 28, wherein the additional treatment is selected from the group consisting of an immune checkpoint inhibitor, a PD-1 inhibitor, and a CTLA-4 inhibitor.

30. The method of claim 22, further comprising administering to the subject an inhibitor of a gene or gene product selected from the group consisting of Lyn, Slc35c1, Lexm, Fam103a1 and Odc.

31. A method of treating cancer in a subject in need thereof, the method comprising administering to the subject a therapeutically effective amount of an inhibitor of a gene or gene product selected from the group consisting of Lyn, Slc35c1, Lexm, Fam103a1 and Odc.

32. The method of claim 31, wherein the inhibitor is selected from the group consisting of an antibody, an siRNA, and a CRISPR system.

33. The method of claim 32, wherein the CRISPR system comprises a Cas9, and at least one sgRNA complementary to a gene selected from the group consisting of Lyn, Slc35c1, Lexm, Fam103a1 and Odc.

34. The method of claim 33, wherein the sgRNA comprises a nucleotide sequence selected from the group consisting of SEQ ID NOs: 821-3020.

35. The method of claim 31, further comprising administering an additional treatment to the subject.

36. The method of claim 35, wherein the additional treatment is selected from the group consisting of an immune checkpoint inhibitor, a PD-1 inhibitor, and a CTLA-4 inhibitor.

37. A method of generating a genetically modified T cell for use in immunotherapy, the method comprising administering to a naïve T cell a vector comprising a first sgRNA complementary to a first nucleotide sequence of a Dhx37 gene and a second sgRNA complementary to a second nucleotide sequence of the Dhx37 gene.

38. The method of claim 37, wherein the first sgRNA nucleotide sequence is selected from the group consisting of SEQ ID NOs: 1-10 and the second sgRNA nucleotide sequence is selected from the group consisting of SEQ ID NOs: 1-10.

39. The method of claim 37, wherein the first sgRNA nucleotide sequence is selected from the group consisting of SEQ ID NOs: 11-820 and the second sgRNA nucleotide sequence is selected from the group consisting of SEQ ID NOs: 11-820.

40. A method of generating a genetically modified T cell for use in immunotherapy, the method comprising administering to a naïve T cell a vector comprising a first sgRNA complementary to a first nucleotide sequence of a gene selected from the group consisting of Lyn, Slc35c1, Lexm, Fam103a1 and Odc and a second sgRNA complementary to a second nucleotide sequence of a gene selected from the group consisting of Lyn, Slc35c1, Lexm, Fam103a1 and Odc.

41. The method of claim 40, wherein the first sgRNA nucleotide sequence is selected from the group consisting of SEQ ID NOs: 821-3020 and the second sgRNA nucleotide sequence is selected from the group consisting of SEQ ID NOs: 821-3020.

42. A composition comprising a genetically modified T cell generated by the method of claim 37.

43. A composition comprising a genetically modified T cell wherein the Dhx37 gene has been mutated.

44. A composition comprising a genetically modified T cell wherein a gene selected from the group consisting of Lyn, Slc35c1, Lexm, Fam103a1 and Odc has been mutated.

45. A composition comprising an inhibitor of Dhx37, wherein the inhibitor is selected from the group consisting of an antibody, an siRNA, and a CRISPR system.

46. The composition of claim 45, wherein the CRISPR system comprises a Cas9, and at least one sgRNA complementary to Dhx37.

47. The composition of claim 46, wherein the sgRNA comprises the nucleotide sequence selected from the group consisting of SEQ ID NOs: 1-10.

48. The composition of claim 46, wherein the sgRNA comprises the nucleotide sequence selected from the group consisting of SEQ ID NOs: 11-820.

49. The composition of claim 45, wherein the antibody recognizes and binds to at least one amino acid sequence selected from the group consisting of SEQ ID NOs: 3022-3031.

50. A kit comprising an inhibitor of Dhx37, wherein the inhibitor is selected from the group consisting of an antibody, an siRNA, and a CRISPR system, and instructional material for use thereof.

51. The kit of claim 50, wherein the CRISPR system comprises a Cas9, and at least one sgRNA complementary to Dhx37.

52. The kit of claim 51, wherein the at least one sgRNA comprises a nucleotide sequence selected from the group consisting of: SEQ ID NOs: 1-10.

53. The kit of claim 51, wherein the at least one sgRNA comprises a nucleotide sequence selected from the group consisting of: SEQ ID NOs: 11-820.

54. A kit comprising a plurality of sgRNAs comprising the nucleotide sequences selected from the group consisting of SEQ ID NOs: 11-3020 and instructional material for use thereof.
